(19) United States
(12) Patent Application Publication
(10) Pub. No.: US 2026/0209324 A1
(43) Pub. Date: Jul. 23, 2026

(54) ANTIBODIES AGAINST EDIL3 AND METHODS OF USE THEREOF

(71) Applicants: DANA-FARBER CANCER INSTITUTE, INC., Boston, MA (US); NONA BIOSCIENCES (SUZHOU) CO., LTD., Suzhou, Jiangsu (CN)

(72) Inventors: F. Stephen Hodi, Boston, MA (US); Saba Tabasum, Boston, MA (US); Chen-lu Li, Suzhou (CN); Jue Zeng, Suzhou (CN); Dinesh Thapa, Lexington, MA (US)

(21) Appl. No.: 19/134,367

(22) PCT Filed: Dec. 7, 2023
(86) PCT No.: PCT/CN2023/137110

§ 371 Date: May 30, 2025

Related U.S. Application Data
(60) Provisional application No. 63/430,840, filed on Dec. 7, 2022.

(51) Int. Cl. C07K 16/22; A61P 35/00; C07K 16/28

(52) U.S. Cl. CPC C07K 16/22; A61P 35/00; C07K 16/2818; C07K 2317/24; C07K 2317/569

(57) ABSTRACT

The present invention is directed to human monoclonal antibodies that bind to epidermal growth factor-like repeats and discoidin I-like domains 3 (EDIL3) and methods of use thereof.

### CROSS-REFERENCE TO RELATED APPLICATIONS

This application claims priority from U.S. Provisional Application No. 63/430,840, filed on Dec. 7, 2022, the entire contents of which is incorporated herein by reference.

### STATEMENT REGARDING FEDERALLY SPONSORED RESEARCH

This invention was made with government support under CA143832 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on [ ], is named [ ] and is [ ] bytes in size.

### BACKGROUND

Tumors create a continuous angiogenic and immunosuppressive microenvironment. There remains a need for therapies that reverse immune exclusion or immune dysfunction in the tumor microenvironment, which could lead to better outcomes for patients. The present disclosure identifies a specific target and corresponding antibodies that can facilitate immune cell traffic in the tumor microenvironment and reverse immune suppressive effects in the tumor microenvironment.

### SUMMARY OF THE INVENTION

Aspects are directed to antibodies or antigen-binding fragments or variants thereof that bind epidermal growth factor-like repeats and discoidin I-like domains 3 (EDIL3) protein. In embodiments, the antibodies or antigen-binding fragments can include a heavy chain variable region (HCVR) and a light chain variable region (LCVR). In embodiments, the HCVR comprises the complementarity determining regions (CDRs) HCDR1, HCDR2 and HCDR3 and the LCVR comprises CDRs LCDR1, LCDR2 and LCDR3. In embodiments, the amino acid sequence of HCDR1 is SYAMS, the amino acid sequence of HCDR2 is AISDSGGSTYYADSVKG, the amino acid sequence of HCDR3 is EGLITFGGVIVIGYFDY, the amino acid sequence of LCDR1 is QASQDISNYLN, the amino acid sequence of LCDR2 is DASNLET, and the amino acid sequence of LCDR3 is QQYDNLPIT. In embodiments, the amino acid sequence of HCDR1 is SYWMS, the amino acid sequence of HCDR2 is NIKQDGSQKYYVDSVKG, the amino acid sequence of HCDR3 is RGNFFFDN, the amino acid sequence of LCDR1 is RASQYVSSYLA, the amino acid sequence of LCDR2 is DASNRAT, and the amino acid sequence of LCDR3 is QQRNNWPPT. In embodiments, the amino acid sequence of HCDR1 is NHYWS, the amino acid sequence of HCDR2 is YIYYSGSTNYNPSLKS, the amino acid sequence of HCDR3 is GFAY, the amino acid sequence of LCDR1 is RASQGITNYLA, the amino acid sequence of LCDR2 is AASTLOS, and the amino acid sequence of LCDR3 is QKYNSAPWT. In embodiments, the amino acid sequence of HCDR1 is SYAMN, the amino acid sequence of HCDR2 is AISGSGDSTYSTDSVKG, the amino acid sequence of HCDR3 is EYYDILTGYWDWYFDL, the amino acid sequence of LCDR1 is RASQSINSNLA, the amino acid sequence of LCDR2 is GASTRAT, and the amino acid sequence of LCDR3 is QQYNNWPLT. In embodiments, the amino acid sequence of HCDR1 is SYGMH, the amino acid sequence of HCDR2 is VMWYDGSDRYSADSVKG, the amino acid sequence of HCDR3 is GYDILTGPDHFDY, the amino acid sequence of LCDR1 is RASQSISSYLN, the amino acid sequence of LCDR2 is AASSLOS, and the amino acid sequence of LCDR3 is QQSYSTPLT. In embodiments, the amino acid sequence of HCDR1 is SYDMN, the amino acid sequence of HCDR2 is TISGSGSHTYYADSVRG, the amino acid sequence of HCDR3 is EGGATAFDI, the amino acid sequence of LCDR1 is RASQGISSYLA, the amino acid sequence of LCDR2 is VASTLQS, and the amino acid sequence of LCDR3 is QQLNNYPT. In embodiments, the amino acid sequence of HCDR1 is TYGMH, the amino acid sequence of HCDR2 is LIWYDGINKYYADSVKG, the amino acid sequence of HCDR3 is PYYDILTGYFDY, the amino acid sequence of LCDR1 is RASQSDSSSYLA, the amino acid sequence of LCDR2 is GTSSRAT, and the amino acid sequence of LCDR3 is QQYGSSPLT. In embodiments, the amino acid sequence of HCDR1 is SYGMH, the amino acid sequence of HCDR2 is VIWYDGTNKYYADSVKG, the amino acid sequence of HCDR3 is DPSLWFGEFPHYYGMDV, the amino acid sequence of LCDR1 is QASQDISNYLN, the amino acid sequence of LCDR2 is DASNLET, and the amino acid sequence of LCDR3 is QQYDNLPLT. In embodiments, the amino acid sequence of HCDR1 is GYYWS, the amino acid sequence of HCDR2 is EIQHSGSTNYKPSLKS, the amino acid sequence of HCDR3 is LTGDSLLFEY, the amino acid sequence of LCDR1 is RASQSVSSYLA, the amino acid sequence of LCDR2 is DTSNRAT, and the amino acid sequence of LCDR3 is QQRSNWPIT. In embodiments, the amino acid sequence of HCDR1 is SYGMH, the amino acid sequence of HCDR2 is VIWYDGSNKYYADSVKG, the amino acid sequence of HCDR3 is DSASDYFDY, the amino acid sequence of LCDR1 is RASQSVSSNLA, the amino acid sequence of LCDR2 is GASTRAT, and the amino acid sequence of LCDR3 is QQYSDWPT. In embodiments, the amino acid sequence of HCDR1 is GYYWS, the amino acid sequence of HCDR2 is EINHSGSTNYKPSLKS, the amino acid sequence of HCDR3 is LTGDSLLFEY, the amino acid sequence of LCDR1 is RASQSVSSYLA, the amino acid sequence of LCDR2 is DTSNRAT, and the amino acid sequence of LCDR3 is QQRSNWPIT.

In embodiments, the antibodies or antigen-binding fragment or variant thereof can include a light chain variable region (LCVR) and a heavy chain variable region (HCVR). In embodiments, the amino acid sequence of the LCVR is DIQMTQSPSSLSASVGDRVTITCQASQDIS NYLNWYQQKPGKAPKLLIYDASNLETG VPSRFSGSGSGTDFTFTISSLQP EDIATYYCQQYDNLPITFGQGTRLEIK, and the amino acid sequence of the HCVR is EVQLLESGGGLVQPGGSLRLSCAASGFTFSSYAMSWVRQAPGKGLEW VSAISDSGG STYYADSVKGRFTISRDNSKNTLYLQMNSLRAEDTAVYYCA QEGLITFGGVIVIGYF DYWGQGTLVTVSS. In embodiments, the amino acid sequence of the LCVR is EIVLTQSPATLSLSPGERATLSCRASQYVSSY LAWYHQKPGQAPRLLIYDASNRATGI PARFSGSGSGTDFTLTISSLEPED FAVYYCQQRNNWPPTFGQGTKVEIK and the amino acid sequence of the HCVR is EVOLVESGGGLVQPGGSLRLSCAASGFTFSSYW MSWVRQAPGKGLEWVANIKQDG SQKYYVDSVKGRFTISRDNAKNSLYLQM NSLRAEDTAVYYCTRRGNFFFDNWGQG TLVTVSS. In embodiments, the amino acid sequence of the LCVR is DIQMTQSPSSLSASVGDRVTITCRASQ GITNYLAWYQQKPGKVPKLLIYAASTLQSG VPSRFSGSGSGTDFTLTINS LQPEDVATYYCQKYNSAPWTFGQGTKVEIK, and the amino acid sequence of the HCVR is QVQLQESGPGLVKPSETLSLTCTVSGGS ISNHYWSWIRQPPGKGLEWIGYIYYSGSTN YNPSLKSRVTISVDTSKNQF SLKLSSVTAADTAVYYCARGFAYWGQGTLVTVSS. In embodiments, the amino acid sequence of the LCVR is EIVMTLSPATLSVSPGERATLSCRASQS INSNLAWYQQKPGQAPRLLIYGASTRATGIP ARFSGSGSGTEFTLTISSL QSEDFAVYYCQQYNNWPLTFGGGTKVEIK, and the amino acid sequence of the HCVR is EVQLLESGGGLGQPGGSLRLSCAASGFTFSSYAMNWVRQAPGKGL EWVSAISGSGD STYSTDSVKGRFTISRDNSKNTLYLQMNSLRAEDTAVYY CAKEYYDILTGYWDWYF DLWGQGTLVTVSS. In embodiments, the amino acid sequence of the LCVR is DIQMTQSPSSLSASVGDRVTITCRASQSISS YLNWYQQKPGKAPKLLIYAASSLQSGV PSRFSGSGSGTDFTLTISSLQPE DFATYYCQQSYSTPLTFGGGTKVEIK, and the amino acid sequence of the HCVR is QVQLVESGGGVVQPGRSLRLSCAASGFTFSSYGMHWVRQAPGKGLEWV AVMWYD GSDRYSADSVKGRFTISRDNSKNTLYLQMNSLRAEDTAVYYCAR GYDILTGPDHFD YWGQGTLVTVSS. In embodiments, the amino acid sequence of the LCVR is DIQLTQSPSFLSASVGDRVTITCRASQGISSYLAWYQ QKPGKAPKLLIYVASTLQSGV PSRFSGSGSGTEFTLTISSLQPEDFATYY CQQLNNYPTFGGGTKVEIK, and the amino acid sequence of the HCVR is EVQLLESGGGLVQPGGSLRLSCAASGFTFSSYDMNWVR QAPGKGPVWVSTISGSGS HTYYADSVRGRFTISRDNSKNTLYLQMNSLRA EDTAVYYCAKEGGATAFDIWGQGT MVTVSS. In embodiments, the amino acid sequence of the LCVR is EIVLTQSPGTLSLSPGERATLSCRASQSDSS SYLAWYQQKPGQAPRLLIYGTSSRATGI SDRFSGSGSGTDFTLTISRLEP EDFAVYYCQQYGSSPLTFGGGTKVEIK, and the amino acid sequence of the HCVR is QVQLVESGGGVVQPGRSLRLSCAASGFTFS TYGMHWVRQAPGKGLEWVALIWYDG INKYYADSVKGRFTISRDNSKNTLY LQMNSLRAEDTAVYFCARPYYDILTGYFDYW GQGTLVTVSS. In embodiments, the amino acid sequence of the LCVR is DIQMTQSPSSLSASVGDRVT ITCQASQDISNYLNWYQQKPGKAPKLLIYDASNLETG VPSRFSGSGSGTDFTFTISSLQPEDIATYYCQQYDNLPLTFGGGTKVEIK, and the amino acid sequence of the HCVR is QVQLVESGGGVVQPGRSLRL SCAASGFTFSSYGMHWVRQAPGKGLEWVAVIWYDG TNKYYADSVKG RFTISRDNSKNTLYLQVNSLRAEDTAVYYCARDPSLWFGEFPHYY GMDVWGQGTTVTVSS. In embodiments, the amino acid sequence of the LCVR is EIVLTQSPATLSLSPGERATLSCRASQSVSSYLAW YQQKPGQAPRLLIYDTSNRATGIP ARFSGSGSGTDFTLTVSSLEPEDFAV YYCQQRSNWPITFGQGTRLEIK, and the amino acid sequence of the HCVR is QVQLQQWGAGLLKPSETLSLTCAVYGGSFSGYYWS WIRQPPGKGLEWIGEIQHSGST NYKPSLKSRVTISVDTSKNQFSLKLSSV TAADTAVYYCAELTGDSLLFEYWGQGTLV TVSS. In embodiments, the amino acid sequence of the LCVR is EIVMTQSPATLSVSPGERATLSCRASQS VSSNLAWYQQKPGQAPRLLIYGASTRATGI PARFSGSGSGTEFTLTISSL QSEDFAVYYCQQYSDWPTFGGGTKVEIR, and the amino acid sequence of the HCVR is QVQLVESGGGVVQPGRSLRLSCAASGFTFSSYGMHWVRQAPGKGLE WVAVIWYDG SNKYYADSVKGRFTISRDTSKNTLYLQMNSLRAEDTAVYYC ARDSASDYFDYWGQ GTLVTVSS. In embodiments, the amino acid sequence of the LCVR is EIVLTQSPATLSLSPGERATLSCRASQSVSSYLAWYQQK PGQAPRLLIYDTSNRATGIP ARFSGSGSGTDFTLTVSSLEPEDFAVYYCQ QRSNWPITFGQGTRLEIK, and the amino acid sequence of the HCVR is QVQLQQWGAGLLKPSETLSLTCAVYGGSFSGYYWSWIRQ PPGKGLEWIGEINHSGST NYKPSLKSRVTISVDTSKNQFSLKLSSVTAAD TAVYYCAELTGDSLLFEYWGQGTLV TVSS.

In embodiments, the antibodies or antigen-binding fragments thereof can include a light chain variable region (LCVR) and a heavy chain variable region (HCVR). In embodiments, the VL region comprises an amino acid sequence that has at least 95% sequence identity to the amino acid sequence DIQMTQSPSSLSASVGDRVTITCQASQDISNYLN WYQQKPGKAPKLLIYDASNLETG VPSRFSGSGSGTDFTFTISSLQPEDIA TYYCQQYDNLPITFGQGTRLEIK, and the VH region comprises an amino acid sequence that has at least 95% sequence identity to the amino acid sequence EVQLLESGGGLVQPGGSLRLSCAASGFTFSSYAMSWVRQAPGKGL EWVSAISDSGG STYYADSVKGRFTISRDNSKNTLYLQMNSLRAEDTAVYY CAQEGLITFGGVIVIGYF DYWGQGTLVTVSS. In embodiments, the VL region comprises an amino acid sequence that has at least 95% sequence identity to the amino acid sequence EIVLTQSPATLSLSPGERATLSCRASQYVS SYLAWYHQKPGQAPRLLIYDASNRATGI PARFSGSGSGTDFTLTISSLEP EDFAVYYCQQRNNWPPTFGQGTKVEIK, and the VH region comprises an amino acid sequence that has at least 95% sequence identity to the amino acid sequence EVOLVESGGGLVQPGGSLRLSCAASGFTFSSYWMSWVRQAP GKGLEWVANIKQDG SQKYYVDSVKGRFTISRDNAKNSLYLQMNSLRAEDT AVYYCTRRGNFFFDNWGQG TLVTVSS. In embodiments, the VL region comprises an amino acid sequence that has at least 95% sequence identity to the amino acid sequence DIQMTQSPSSLSASVGDRVTITCRASQGI TNYLAWYQQKPGKVPKLLIYAASTLQSG VPSRFSGSGSGTDFTLTINSLQ PEDVATYYCQKYNSAPWTFGQGTKVEIK, and the VH region comprises an amino acid sequence that has at least 95% sequence identity to the amino acid sequence QVQLQESGPGLVKPSETLSLTCTVSGGSISNHYWSWIRQP PGKGLEWIGYIYYSGSTN YNPSLKSRVTISVDTSKNQFSLKLSSVTAADT AVYYCARGFAYWGQGTLVTVSS. In embodiments, the VL region comprises an amino acid sequence that has at least 95% sequence identity to the amino acid sequence EIVMTLSPATLSVSPGERATLSCRASQSINSNLA WYQQKPGQAPRLLIYGASTRATGIP ARFSGSGSGTEFTLTISSLQSEDFA VYYCQQYNNWPLTFGGGTKVEIK, and the VH region comprises an amino acid sequence that has at least 95% sequence identity to the amino acid sequence EVQLLESGGGLGQPGGSLRLSCAASGFTFSSYAMNWVRQAPGKGL EWVSAISGSGD STYSTDSVKGRFTISRDNSKNTLYLQMNSLRAEDTAVYY CAKEYYDILTGYWDWYF DLWGQGTLVTVSS. In embodiments, the VL region comprises an amino acid sequence that has at least 95% sequence identity to the amino acid sequence DIQMTQSPSSLSASVGDRVTITCRA SQSISSYLNWYQQKPGKAPKLLIYAASSLQSGV PSRFSGSGSGTDFTLTI SSLQPEDFATYYCQQSYSTPLTFGGGTKVEIK, and the VHregion comprises an amino acid sequence that has at least 95% sequence identity to the amino acid sequence QVQLVESGGGVVQPGRSLRLSCAASGFTFSSYGMHWV RQAPGKGLEWVAVMWYD GSDRYSADSVKGRFTISRDNSKNTLYLQMNSLR AEDTAVYYCARGYDILTGPDHFD YWGQGTLVTVSS. In embodiments, the VL region comprises an amino acid sequence that has at least 95% sequence identity to the amino acid sequence DIQLTQSPSFLSASVGDRVTITCRASQGISSYLAWYQQ KPGKAPKLLIYVASTLQSGV PSRFSGSGSGTEFTLTISSLQPEDFATYYC QQLNNYPTFGGGTKVEIK, and the VH region comprises an amino acid sequence that has at least 95% sequence identity to the amino acid sequence EVQLLESGGGLVQPGGSLRLSCAASGFTFSSYDMNWVRQAPGKGPVWVST ISGSGS HTYYADSVRGRFTISRDNSKNTLYLQMNSLRAEDTAVYYCAKEG GATAFDIWGQGT MVTVSS. In embodiments, the VL region comprises an amino acid sequence that has at least 95% sequence identity to the amino acid sequence EIVLTQSPGTLSLSPGERATLSCRASQSDSSSYLAWYQQKPGQAPRLLIYGTSS RATGI SDRFSGSGSGTDFTLTISRLEPEDFAVYYCQQYGSSPLTFGGGTKVEIK, and the VH region comprises an amino acid sequence that has at least 95% sequence identity to the amino acid sequence QVQLVESGGGVVQPGRSLRLSCAASGFTFS TYGMHWVRQAPGKGLEWVALIWYDG INKYYADSVKGRFTISRDNSKNTLY LQMNSLRAEDTAVYFCARPYYDILTGYFDYW GQGTLVTVSS. In embodiments, the VL region comprises an amino acid sequence that has at least 95% sequence identity to the amino acid sequence DIQMTQSPSSLSASVGDRVTITCQASQDISN YLNWYQQKPGKAPKLLIYDASNLETG VPSRFSGSGSGTDFTFTISSLQPE DIATYYCQQYDNLPLTFGGGTKVEIK, and the VH region comprises an amino acid sequence that has at least 95% sequence identity to the amino acid sequence QVQLVESGGGVVQPGRSLRLSCAASGFTFSSYGMHWVRQAPG KGLEWVAVIWYDG TNKYYADSVKGRFTISRDNSKNTLYLQVNSLRAEDTA VYYCARDPSLWFGEFPHYY GMDVWGQGTTVTVSS. In embodiments, the VL region comprises an amino acid sequence that has at least 95% sequence identity to the amino acid sequence EIVLTQSPATLSLSPGERATLSCRASQSVSSYLAWYQQ KPGQAPRLLIYDTSNRATGIP ARFSGSGSGTDFTLTVSSLEPEDFAVYYC QQRSNWPITFGQGTRLEIK, and the VH region comprises an amino acid sequence that has at least 95% sequence identity to the amino acid sequence QVQLQQWGAGLLKPSETLSLTCAVYGGSFSGYYWSWIRQPPGKGLEWIG EIQHSGST NYKPSLKSRVTISVDTSKNQFSLKLSSVTAADTAVYYCAELTGDS LLFEYWGQGTLV TVSS. In embodiments, the VL region comprises an amino acid sequence that has at least 95% sequence identity to the amino acid sequence EIVMTQSPATLSVSPGERATLSCRASQSVSSNLAWYQQK PGQAPRLLIYGASTRATGI PARFSGSGSGTEFTLTISSLQSEDFAVYYCQ QYSDWPTFGGGTKVEIR, and the VH region comprises an amino acid sequence that has at least 95% sequence identity to the amino acid sequence QVQLVESGGGVVQPGRSLRLSCAASGFTFSSYGMHWVRQAPGKGLEWVAVI WYDG SNKYYADSVKGRFTISRDTSKNTLYLQMNSLRAEDTAVYYCARDSA SDYFDYWGQ GTLVTVSS. In embodiments, the VL region comprises an amino acid sequence that has at least 95% sequence identity to the amino acid sequence EIVLTQSPATLSLSPGERATLSCRASQSVSSYLAWYQQ KPGQAPRLLIYDTSNRATGIP ARFSGSGSGTDFTLTVSSLEPEDFAVYYC QQRSNWPITFGQGTRLEIK, and the VH region comprises an amino acid sequence that has at least 95% sequence identity to the amino acid sequence QVQLQQWGAGLLKPSETLSLTCAVYGGSFSGYYWSWIRQPPGKGLEWIG EINHSGST NYKPSLKSRVTISVDTSKNQFSLKLSSVTAADTAVYYCAELT GDSLLFEYWGQGTLV TVSS.

In embodiments, the fragment of the antibodies or antigen-binding fragments or variants thereof can include an F (ab), an Fv, or an scFv. In embodiments, the fragment of the antibodies or antigen-binding fragments or variants thereof can include a VhH.

Aspects are directed to a therapeutic antibody that binds to an epidermal growth factor-like repeats and discoidin I-like domains 3 (EDIL3) protein comprising a variable domain and a constant domain. In embodiments, the constant domain is IgG and the variable domain comprises a framework region and a complementary determining means for binding to an epidermal growth factor-like repeats and discoidin I-like domains 3 (EDIL3) protein. In embodiments, the constant region of the therapeutic antibody isIgG1. In embodiments, the therapeutic antibody can include any one of the antibodies according to Table 1-Table 4.

Aspects are directed to a pharmaceutical composition that can include any of the antibodies described herein, and one or more pharmaceutically acceptable carriers, diluents, or excipients.

Aspects are directed to methods of treating cancer in a subject that can include administering to a subject in need thereof an effective amount of any of the antibodies described herein, or the pharmaceutical composition described herein. In embodiments, the method of treating cancer can include administering to a subject an angiogenesis inhibitor, a checkpoint blockade inhibitor, or a combination thereof. In embodiments, the angiogenesis inhibitor comprises bevacizumab. In embodiments, the checkpoint blockade inhibitor comprises ipilimumab.

Aspects are directed to the use of any of the antibodies described herein, or the pharmaceutical composition described herein for treating cancer. In some embodiments, any of the antibodies described herein, or any of the pharmaceutical compositions described herein can be use in treating cancer.

Aspects are directed to methods of decreasing immune suppressive effects of cancer-associated fibroblasts in a subject, methods that can include the administration to the subject an effective amount of any of the antibodies described herein, or any of the pharmaceutical compositions described herein.

Aspects are directed to the use of any of the antibodies described herein, or any of the pharmaceutical compositions described herein for decreasing the immune suppressive effects of cancer-associated fibroblasts.

Aspects are directed to any of the antibodies described herein, or any of the pharmaceutical compositions described herein for use in decreasing the immune suppressive effects of cancer-associated fibroblasts.

Aspects are directed to nucleic acids encoding any of the antibodies described herein. In embodiments, the nucleic acid sequence encoding HCDR1 is AGCTATGCCATGAGC or a degenerate variant thereof; the nucleic acid sequence encoding HCDR2 is GCTATTAGTGATAGTGGTGGTAGCACATACTA CGCAGACTCCGTGAAGGGC, or a degenerate variant thereof, the nucleic acid sequence encoding HCDR3 is GAGGGTTTGATTACGTTTGGG GGAGTTATCGTTATAGGCTACTTTGACTAC, or a degenerate variant thereof, the nucleic acid sequence encoding LCDR1 is CAGGCGAGTCAGGACATTAGCAACTATTTAAAT, or a degenerate variant thereof, the nucleic acid sequence encoding LCDR2 is GATGCATCCAATTTGGA AACA, or a degenerate variant thereof, and the nucleic acid sequence encoding LCDR3 is CAACAGTATGATAATCTCCCGATCACC, or a degenerate variant thereof. In embodiments, the nucleic acid sequence encoding HCDR1 is AGCTATTGGATGAGC, or a degenerate variant thereof, the nucleic acid sequence encoding HCDR2 is AATATAAAGCAAGATGGAAGTCAGAAATACTATGTGGACTCTGTGAAGGGC, or a degenerate variant thereof, the nucleic acid sequence encoding HCDR3 is CGTGGGAACTTCTTCTTTGACAAT, or a degenerate variant thereof, the nucleic acid sequence encoding LCDR1 is AGGGCCAGTCAGTATGTTAGCAGCTACTTAGCC, or a degenerate variant thereof, the nucleic acid sequence encoding LCDR2 is GATGCATCCAACAGGGCCACT, or a degenerate variant thereof, and the nucleic acid sequence encoding LCDR3 is CAGCAGCGTAACAACTGGCCTCCGACG, or a degenerate variant thereof. In embodiments, the nucleic acid sequence encoding HCDR1 is AATCACTACTGGAGC, or a degenerate variant thereof, the nucleic acid sequence encoding HCDR2 is TATATCTATTACAGTGGGAGCACCAACTACAACCCCTCCCTCAAGAGT, or a degenerate variant thereof, the nucleic acid sequence encoding HCDR3 is GGGTTTGCTTAC, or a degenerate variant thereof, the nucleic acid sequence encoding LCDR1 is CGGGCGAGTCAGGGCATTACCAATTATTTAGCC, or a degenerate variant thereof, the nucleic acid sequence encoding LCDR2 is GCTGCATCCACTTTGCAATCA, or a degenerate variant thereof, and the nucleic acid sequence encoding LCDR3 is CAAAAGTATAACAGTGCCCCGTGGACG, or a degenerate variant thereof. In embodiments, the nucleic acid sequence encoding HCDR1 is AGCTATGCCATGAAC, or a degenerate variant thereof, the nucleic acid sequence encoding HCDR2 is GCTATCAGTGGCAGTGGTGATAGCACATACTCCACAGACTCCGTGAAGGGC, or a degenerate variant thereof, the nucleic acid sequence encoding HCDR3 is GAGTATTACGATATTTTGACTGGTTATTGGGACTGGTACTTCGATCTC, or a degenerate variant thereof, the nucleic acid sequence encoding LCDR1 is AGGGCCAGTCAGAGTATTAACAGCAACTTAGCC, or a degenerate variant thereof, the nucleic acid sequence encoding LCDR2 is GGTGCATCCACCAGGGCCACT, or a degenerate variant thereof, and the nucleic acid sequence encoding LCDR3 is CAGCAGTATAATAACTGGCCGCTCACT, or a degenerate variant thereof. In embodiments, the nucleic acid sequence encoding HCDR1 is AGCTATGGCATGCAT, or a degenerate variant thereof, the nucleic acid sequence encoding HCDR2 is GTTATGTGGTATGATGGAAGTGATAGATACTCTGCAGACTCCGTGAAGGGC, or a degenerate variant thereof, the nucleic acid sequence encoding HCDR3 is GGGTACGATATTTTGACTGGTCCCGACCACTTTGACTAC, or a degenerate variant thereof, the nucleic acid sequence encoding LCDR1 is CGGGCAAGTCAGAGCATTAGCAGTTATTTAAAT, or a degenerate variant thereof, the nucleic acid sequence encoding LCDR2 is GCTGCATCCAGTTTGCAAAGT, or a degenerate variant thereof, and the nucleic acid sequence encoding LCDR3 is CAACAGAGTTACAGTACCCCGCTCACT, or a degenerate variant thereof. In embodiments, the nucleic acid sequence encoding HCDR1 is AGCTATGACATGAAC, or a degenerate variant thereof, the nucleic acid sequence encoding HCDR2 is ACTATTAGTGGTAGTGGTAGTCACACATACTACGCAGACTCCGTGAGGGGC, or a degenerate variant thereof, the nucleic acid sequence encoding HCDR3 is GAGGGGGGAGCTACTGCTTTTGATATC, or a degenerate variant thereof, the nucleic acid sequence encoding LCDR1 is CGGGCCAGTCAGGGCATTAGCAGTTATTTAGCC, or a degenerate variant thereof, the nucleic acid sequence encoding LCDR2 is GTTGCATCCACTTTGCAAAGT, or a degenerate variant thereof, and the nucleic acid sequence encoding LCDR3 is CAACAGCTTAATAATTACCCCACT, or a degenerate variant thereof. In embodiments, the nucleic acid sequence encoding HCDR1 is ACCTATGGCATGCAC, or a degenerate variant thereof, the nucleic acid sequence encoding HCDR2 is CTTATATGGTATGATGGAATTAATAAATACTATGCGGACTCCGTGAAGGGC, or a degenerate variant thereof, the nucleic acid sequence encoding HCDR3 is CCCTATTACGATATTTTGACTGGTTATTTTGACTAC, or a degenerate variant thereof, the nucleic acid sequence encoding LCDR1 is AGGGCCAGTCAGAGTGATAGCAGCAGCTACTTAGCC, or a degenerate variant thereof, the nucleic acid sequence encoding LCDR2 is GGTACATCCAGTAGGGCCACT, or a degenerate variant thereof, and the nucleic acid sequence encoding LCDR3 is CAGCAGTATGGTAGCTCACCGCTCACT, or a degenerate variant thereof. In embodiments, the nucleic acid sequence encoding HCDR1 is AGCTATGGCATGCAC, or a degenerate variant thereof, the nucleic acid sequence encoding HCDR2 is GTTATATGGTATGATGGAACTAATAAATACTATGCAGACTCCGTGAAGGGC, or a degenerate variant thereof, the nucleic acid sequence encoding HCDR3 is GATCCCTCCTTATGGTTCGGGGAGTTCCCTCATTACTACGGTATGGACGTC, or a degenerate variant thereof, the nucleic acid sequence encoding LCDR1 is CAGGCGAGTCAGGACATTAGCAATTATTTAAAT, or a degenerate variant thereof, the nucleic acid sequence encoding LCDR2 is GATGCATCCAATTTGGAAACA, or a degenerate variant thereof, and the nucleic acid sequence encoding LCDR3 is CAACAGTATGATAATCTCCCGCTCACT, or a degenerate variant thereof. In embodiments, the nucleic acid sequence encoding HCDR1 is GGTTACTACTGGAGC, or a degenerate variant thereof, the nucleic acid sequence encoding HCDR2 is GAAATCCAACATAGTGGAAGCACCAACTACAAACCGTCCCTCAAGAGT, or a degenerate variant thereof, the nucleic acid sequence encoding HCDR3 is CTAACTGGGGATTCCCTTTTGTTTGAGTAC, or a degenerate variant thereof, the nucleic acid sequence encoding LCDR1 is AGGGCCAGTCAGAGTGTTAGCAGCTACTTAGCC, or a degenerate variant thereof, the nucleic acid sequence encoding LCDR2 is GATACATCCAACAGGGCCACT, or a degenerate variant thereof, and the nucleic acid sequence encoding LCDR3 is CAGCAGCGTAGCAACTGGCCGATCACC, or a degenerate variant thereof. In embodiments, the nucleic acid sequence encoding HCDR1 is AGCTATGGCATGCAC, or a degenerate variant thereof, the nucleic acid sequence encoding HCDR2 is GTTATATGGTATGATGGAAGTAATAAATACTATGCAGACTCCGTGAAGGGC, or a degenerate variant thereof, the nucleic acid sequence encoding HCDR3 is GATAGCGCCTCCGACTACTTTGACTAC, or a degenerate variant thereof, the nucleic acid sequence encoding LCDR1 is AGGGCCAGTCAGAGTGTTAGCAGCAACTTAGCC, or a degenerate variant thereof, the nucleic acid sequence encoding LCDR2 GGTGCATCCACCAGGGCCACT, or a degenerate variant thereof, and the nucleic acid sequence encoding LCDR3 is CAGCAGTATAGTGACTGGCCCACT, or a degenerate variant thereof. In embodiments, the nucleic acid sequence encoding HCDR1 is GGTTACTACTGGAGC, or a degenerate variant thereof, the nucleic acid sequence encoding HCDR2 is GAAATCAATCATAGTGGAAGCACCAACTACAAACCGTCCCTCAAGAGT, or a degenerate variant thereof, the nucleic acid sequence encoding HCDR3 is CTAACTGGGGATTCCCTTTTGTTTGAGTAC, or a degenerate variant thereof, the nucleic acid sequence encoding LCDR1 is AGGGCCAGTCAGAGTGTTAGC AGCTACTTAGCC, or a degenerate variant thereof, the nucleic acid sequence encoding LCDR2 is GATACATCCAACAGGGCCACT, or a degenerate variant thereof, and the nucleic acid sequence encoding LCDR3 is CAGCAGCGTAGCAACTGGCCGATCACC, or a degenerate variant thereof.

In embodiments, the nucleic acid sequence encoding LCVR is GACATCCAGATGACCCAGTCTCCATCCTCCCTGTCTGCATCTG TAGGAGACAGAGTCACCATCACTTGCCAGGCGAG TCAGGACATTAGCAACTATTTAAATTGGTATCAG CAGAAACCAGGGAAAGCCCCTAAGCTCCTGATCTACGATGCATCCAATTTG GA AACAGGGGTCCCATCAAGGTTCAGTGGAAGTGGATCTGGGACAGATTT TACTTTCACCATCAGCAGCCTGCAGCCTGAAGATATTGCAACATATTACTG TCAACAGTATGATAATCTCCCGATCACCTTCGGCCAAGGGACACGACTGGA GATTAAA, or a degenerate variant thereof, and the nucleic acid sequence encoding HCVR is GAGGTGCAGCTGTTGGAGTCTGGGGGAGGC TTGGTACAGCCTGGGGGGTCCCTGAGACTCTCCTGTGCAGCCTCTGGATTC ACCTTTAGCAGCTATGCCATGAGCTGGGTCCGCC AGGCTCCAGGGAAGGGGCTGGAGTGGGTCTCAGC TATTAGTGATAGTGGTGGTAGCACATACTACGCAGACTCCGTGAAGGGCCG GTTCACCATCTCCAGAGACAATTCCAAGAACACGCTGTATCTGCAAATGAA CAGCCTGAGAGCCGAGGACACGGCCGTATATTAC TGTGCGCAGGAGGGTTTGATTACGTTTGGGGGAGTTATCGTTATAGGCTAC TTTGACTACTGGGGCCAGGGAACCCTGGTCACCGTCTCCTCA, or a degenerate variant thereof. In embodiments, the nucleic acid sequence encoding LCVR is GAAATTGTGTTGACACAGTCTCCAGCCACCCTGT CTTTGTCTCCAGGGGAAAGAGCCACCCTCTCCTGCAGGGCCAGTCAGTATG TTAGCAGCTACTTAGCCTGGTACCACCAGAAACCTGGCCAGGCTCCCAGGC TCCTCATCTACGATGCATCCAACAGGGCCACTGGCATCCCAGCCAGGTTCA GTGGCAGTGGGTCTGGGACAGACTTCACTCTCACCATCAGCAGCCTAGAGC CTGAAGATTTTGCAGTTTATTACTGTCAGCAGCGTAACAACTGGCCTCCGA CGTTCGGCCAAGGGACCAAGGTGGAAATCAAA, or a degenerate variant thereof, and the nucleic acid sequence encoding HCVR is GAGGTGCAGCTGGTGGAGTCTGGGGGAGGCTTGGTCCAG CCTGGGGGGTCCCTGAGACTCTCCTGTGCAGCCTCTGGATTCACCTTTAGT AGCTATTGGATGAGCTGGG TCCGCCAGGCTCCAGGGAAGGGGCTGGA ATGGGTGGCCAATATAAAGCAAGATGGAAGTCAG AAATACTATGTGGACTCTGTGAAGGGCCGATTCACCATCTCCAGAGACAAC GCCAAGAACTCACTGTATCTGCAAATGAACAGCC TGAGAGCCGAGGACACGGCTGTGTATTACTGTACGAGACGTGGGAACTTCT TCTTTGACAATTGGGGCCAGGGAACCCTGGTCACCGTCTCCTCA, or a degenerate variant thereof. In embodiments, the nucleic acid sequence encoding LCVR is GACATCCAGATGACCCAGTCTCCATCCTCCCTGTCTGCATCTGTAGGAG ACAGAGTCACCATCACTTGCCGGGCGAGTCAGGGCATTACCAATTATTTAG CCTGGTATCAGCAGAAACCAGGGAAAGTTCCTAAGCTCCTGATCTATGCTG CATCCACTTTGCAATCAGGGGTCCCATCTCGGTTCAGTGGCAGTGGATCTG GGACAGATTTCACTCTCACCATCAACAGCCTGCAGCCTGAAGATGTTGCAA CTTATTACTGTCAAAAGTATAACAGTGCCCCGTGGACGTTCGGCCAAGGGA CCAAGGTGGAAATCAAA, or a degenerate variant thereof, and the nucleic acid sequence encoding HCVR is CAGGTGCAGCTGCAGGAGTC GGGCCCAGGACTGGTGAAGCCTTCGGAGACCCTG TCCCTCACTTGCACTGTCTCTGGTGGCTCCATCAGTAATCACTACTGGAGC TGGATTCGGCAGCCCCCAGGGAAGGGACTGGAGTGGATTGGGTATATCTAT TACAGTGGGAGCACCAACTACAACCCCTCCCTCAAGAGTCGAGTCACCATA TCAGTAGACACGTCCAAGAACCAGTTCTCCCTGAAGCTGAGCTCTGTGACC GCTGCGGACACGGCCGTGTATTACTGTGCGAGGGGGTTTGCTTACTGGGGC CAAGGCACTCTGGTCACTGTCTCTTCA, or a degenerate variant thereof. In embodiments, the nucleic acid sequence encoding LCVR is GAAATAGTGATGACGCTGTCTCCAGCCACCCTGTCTGTGTCTCCAGGGG AAAGAGCCACCCTCTCCTGCAGGGCCAGTCAGAGTATTAACAGCAACTTAG CCTGGTACCAGCAGAAACCTGGCCAGGCTCCCAGGCTCCTCATCTATGGTG CATCCACCAGGGCCACTGGTATCCCAGCCAGGTTCAGTGGCAGTGGGTCTG GGACAGAGTTCACTCTCACCATCAGCAGCCTGCAGTCTGAAGATTTTGCAG TTTATTACTGTCAGCAGTATAATAACTGGCCGCTCACTTTCGGCGGAGGGA CCAAGGTGGAGATCAAA, or a degenerate variant thereof, and the nucleic acid sequence encoding HCVR is GAGGTGCAGCTGTTGGAATC TGGGGGAGGCTTGGGACAGCCTGGGGGGTCCCTGAGACTCTCCTGTGCAGC CTCTGGATTCACCTTTAGCAGCTATGCCATGAACTGGGTCCGCCAGGCTCC AGGGAAGGGGCTGGAGTGGGTCTCAGCTATCAGTGGCAGTG GTGATAGCACATACTCCACAGACTCCGTGAAGGGCCGGTTCACCA TCTCCAGAGACAATTCCAAGAACACGCTGTATCTGCAAATGAACAGCCTGA GAGCCGAGGACACGGCCGTTTATTACTGTGCGAAAGAGTATTACGATATTT TGACTGGTTATTGGGACTGGTACTTCGATCTCTGGGGCCAAGGCACCCTGG TCACTGTCTCCTCA, or a degenerate variant thereof. In embodiments, the nucleic acid sequence encoding LCVR is GACATCCAGATGACCCAGTCTCCATCCT CCCTGTCTGCATCTGTAGGAGACAGAGTCACCAT CACTTGCCGGGCAAGTCAGAGCATTAGCAGTTAT TTAAATTGGTATCAGCAGAAACCAGGGAAAGCCC CTAAGCTCCTGATCTATGCTGCATCCAGTTTGCAAAGTGGGGTCCCATCGA GGTTCAGTGGCAGTGGATCTGGGACAGATTTCACTCTCACCATCAGCAGTC TGCAACCTGAAGATTTTGCAACTTACTATTGTCAACAGAGTTACAGTACCC CGCTCACTTTCGGCGGAGGGACCAAGGTGGAGATCAAA, or a degenerate variant thereof, and the nucleic acid sequence encoding HCVR is CAGGTGCAGCTGGTGGAGTCTGGGGGAGGCGTG GTCCAGCCTGGGAGGTCCCTGAGACTCTCCTGTGCAGCGTCTGGATTCACC TTCAGTAGCTATGGCATGCATTGGGTCCGCCAGGCTCCAGGCAAGGGGCTG GAGTGGGTGGCAGTTATGTGGTATGATGGAAGTGATAGATACTCTGCAGAC TCCGTGAAGGGCCGATTCACCATCTCCAGAGACAATTCCAAGAACACGCTG TATTTGCAAATGAACAGCCTGAGAGCCGAGGACACGGCTGTGTATTACTGT GCGAGGGGGTACGATATTTTGACTGGTCCCGACCACTTTGACTACTGGGGC CAGGGAACCCTGGTCACCGTCTCCTCA, or a degenerate variant thereof. In embodiments, the nucleic acid sequence encoding LCVR is GACATCCAGTTGACCCAGTCTCCATCCTTCCTGTCTGCATCTGTAGGAG ACAGAGTCACCATCACTTGCCGGGCCAGTCAGGGCATTAGCAGTTATTTAG CCTGGTATCAGCAAAAACCAGGGAAAGCCCCTAAGCTCCTGATCTATGTTG CATCCACTTTGCAAAGTGGGGTCCCATCAAGGTTCAGCGGCAGTGGATCTG GGACAGAATTCACTCTCACAATCAGCAGCCTGCAGCCTGAAGATTTTGCAA CTTATTACTGTCAACAGCTTAATAATTACCCCACTTTCGGCGGAGGGACCA AGGTGGAGATCAAA, or a degenerate variant thereof, and the nucleic acid sequence encoding HCVR is GAAGTACAGTTGTTGGAGTCTGG GGGAGGCTTGGTACAGCCTGGGGGGTCCCTGAGACTCTCCTGTGCAGCCTC TGGATTCACCTTTAGCAGCTATGACATGAACTGGGTCCGCCAGGCTCCAGG GAAGGGGCCGGTGTGGGTCTCAACTATTAGTGGTAGTGGTAGTCACACATA CTACGCAGACTCCGTGAGGGGCCGGTTCACCATCTCCAGAG ACAACTCCAAGAACACGCTGTATCTGCAAATGAACAGCCTGAGAG CCGAGGACACGGCCGTATATTACTGTGCGAAAGA GGGGGGAGCTACTGCTTTTGATATCTGGGGCCAAGGGACAATGGTCACCGT CTCTTCA, or a degenerate variant thereof. In embodiments, the nucleic acid sequence encoding LCVR is GAAATTGTGTTGACGCAG TCTCCAGGCACCCTGTCTTTGTCTCCAGGGGAAAGAGCCACCCTCTCCTGC AGGGCCAGTCAGAGTGATAGCAGCAGCTACTTAG CCTGGTATCAGCAGAAACCTGGCCAGGCTCCCAGGCTCCTCATATATGGTA CATCCAGTAGGGCCACTGGCATCTCAGACAGGTTCAGTGGCAGTGGGTCTG GGACAGACTTCACTC TCACCATCAGCAGACTGGAGCCTGAAGATTTTGCA GTGTATTACTGTCAGCAGTATGGTAGCTCACCGCTCACTTTCGGCGGAGGG ACCAAGGTGGAGATCAAA, or a degenerate variant thereof, and the nucleic acid sequence encoding HCVR is CAGGTGCAGCTGGTGGAGT CTGGGGGAGGCGTGGTCCAGCCTGGGAGGTCCCTGAGACTCTCCTGTGCAG CGTCTGGATTCACCTTCAGTACCTATGGCATGCACTGGGTCCGCCAGGCTC CAGGCAAGGGGCTGGAGTGGGTGGCACTTATATG GTATGATGGAATTAATAAATACTATGCGGACTCCGTGAAGGGCCGATTCAC CATCTCCAGAGACAATTCCAAGAACACGCTGTATCTGCAAATGAACAGCCT GAGAGCCGAGGACACGGCTGTGTATTTCTGTGCGAGACCCTATTACGATAT TTTGACTGGTTATTTTGACTACTGGGGCCAGGGAACCCTGGTCACCGTCTC CTCA, or a degenerate variant thereof. In embodiments, the nucleic acid sequence encoding LCVR is GACATCCAGATGACCCAGTCT CCATCCTCCCTGTCTGCATCTGTAGGAGACAGAGTCACCATCACTTGCCAG GCGAGTCAGGACATTAGCAATTATTTAAATTGGT ATCAGCAGAAACCAGGGAAAGCCCCTAAGCTCCT GATCTACGATGCATCCAATTTGGAAACAGGGGTCCCATCAAGGTTCAGTGG AAGTGGATCTGGGACAGATTTTACTTTCACCATCAGCAGCCTGCAGCCTGA AGATATTGCAACATATTACTGTCAACAGTATGATAATCTCCCGCTCACTTT CGGCGGAGGGACCAAGGTGGAGATCAAA, or a degenerate variant thereof, and the nucleic acid sequence encoding HCVR is CAGGTGCAGCTGGTGGAGTCTGGGGGAGGCGTGGTCCAGCCTG GGAGGTCCCTGAGACTCTCCTGTGCAGCGTCTGGATTCACCTTCAGTAGCT ATGGCATGCACTGGGTCCGCCAGGCTCCAGGCAA GGGGCTGGAGTGGGTGGCAGTTATATGGTATGAT GGAACTAATAAATACTATGCAGACTCCGTGAAGGGCCGATTCACCATCTCC AGAGACAATTCCAAGAACACGCTGTATCTGCAAG TGAACAGCCTGAGAGCCGAGGACA CGGCTGTGTAT TACTGTGCGAGAGATCCCTCCTTATGGTTCGGGGAGTTCCCTCATTACTAC GGTATGGACGTCTGGGGCCAAGGGACCACGGTCACCGTCTCCTCA, or a degenerate variant thereof. In embodiments, the nucleic acid sequence encoding LCVR is GAAATTGTGTTGACACAGTCTCCAGCCACCCTGTCTTTGTCTCCAGGG GAAAGAGCCACCCTCTCCTGCAGGGCCAGTCAGAGTGTTAGCAGCTACTTA GCCTGGTACCAACAGAAACCTGGCCAGGCTCCCAGGCTCCTCATCTATGAT ACATCCAACAGGGCCACTGGCATCCCAGCCAGGTTCAGTGGCAGTGGGTCT GGGACAGACTTCACTCTCACCGTCAGCAGCCTAGAGCCTGAAGATTTTGCA GTTTATTACTGTCAGCAGCGTAGCAACTGGCCGATCACCTTCGGCCAAGGG ACACGACTGGAGATTAAA, or a degenerate variant thereof, and the nucleic acid sequence encoding HCVR is CAGGTGCAGCTACAGCAGT GGGGCGCAGGACTGTTGAAGCCTTCGGAGACCCTGTCCCTCACCTGCGCTG TCTATGGTGGGTCCTTCAGTGGTTACTACTGGAGCTGGATACGCCAGCCCC CAGGGAAGGGGCTGGAGTGGATTGGGGAAATCCA ACATAGTGGAAGCACCAACTACAAACCGTCCCTC AAGAGTCGAGTCACCATATCAGTAGACACGTCCAAGAACCAGTTCTCCCTG AAGCTGAGCTCTGTGACCGCCGCGGACACGGCTGTGTATTACTGTGCGGAG CTAACTGGGGATTCCCTTTTGTTTGAGTACTGGGGCCAGGGAACCCTGGTC ACCGTCTCCTCA, or a degenerate variant thereof. In embodiments, the nucleic acid sequence encoding LCVR is GAAATAGTGATGACGCAGTCTCCAGCCACC CTGTCTGTGTCTCCAGGGGAAAGAGCCACCCTCTCCTGCAGGGCCAGTCAG AGTGTTAGCAGCAACTTAGCCTGGTACCAGCAGAAACCTGGCCAGGCTCCC AGGCTCCTCATCTATGGTGCATCCACCAGGG CCACTGGTATCCCAGCCAG ATTCAGTGGCAGTGGGTCTGGGACAGAGTTCACTCTCACCATCAGCAGCCT GCAGTCTGAAGATTTTGCAGTTTATTACTGTCAGCAGTATAGTGACTGGCC CACTTTCGGCGGAGGGACCAAGGTGGAGATCAGA, or a degenerate variant thereof, and the nucleic acid sequence encoding HCVR is CAGGTGCAGCTGGTGGAGTCTGGGGGAGGCGTGGTCC AGCCTGGGAGGTCCCTG AGACTCTCCTGTGCAGCGTCTGGATTCACCTTC AGTAGCTATGGCATGCACTGGGTCCGCCAGGCTC CAGGCAAGGGGCTGGAGTGGGTGGCAGTTATATG GTATGATGGAAGTAATAAATACTATGCAGACTCCGTGAAGGGCCGATTCAC CATCTCCAGAGACACTTCCAAGAACACACTGTATCTGCAAATGAACAGCCT GAGAGCCGAGGACACGGCTGTGTATTACTGTGCG AGAGATAGCGCCTCCGACTACTTTGACTACTGGGG CCAGGGAACCC TGGTCACCGTCTCCTCA, or a degenerate variant thereof. In embodiments, the nucleic acid sequence encoding the LCVR is GAAATTGTGTTGACACAGTCT CCAGCCACCCTGTCTTTGTCTCCAGGGGAAAGAGCCACCCTCTCCTGCAGG GCCAGTCAGAGTGTTAGCAGCTACTTAGCCTGGTACCAACAGAAACCTGGC CAGGCTCCCAGGCTCCTCATCTATGATACATCCAACAGGGCCACTGGCATC CCAGCCAGGTTCAGTGGCAGTGGGTCTGGGACAGACTTCACTCTCACCGTC AGCAGCCTAGAGCCTGAAGATTTTGCAGTTTATTACTGTCAGCAGCGTAGC AACTGGCCGATCACCTTCGGCCAAGGGACACGACTGGAGATTAAA, or a degenerate variant thereof, and the nucleic acid sequence encoding HCVR is CAGGTGCAGCTACAGCAGTGGGGCGC AGGACTGTTGAAGCCTTCGGAGACCCTGTCCCTCACCTGCGCTGTCTATGG TGGGTCCTTCAGTGGTTACTACTGGAGCTGGATACGCCAGCCCCCAGGGAA GGGGCTGGAGTGGATTGGGGAAATCAATCATAGT GGAAGCACCAACTACAAACCGTCCCTCAAGAGTC GAGTCACCATATCAGTAGACACGTCCAAGAACCAGTTCTCCCTGAAGCTGA GCTCTGTGACCGCCGCGGACACGGCTGTGTATTACTGTGCGGAGCTAACTG GGGATTCCCTTTTGTTTGAGTACTGGGGCCAGGGAACCCTGGTCACCGTCT CCTCA, or a degenerate variant thereof.

Aspects are directed to vector comprising the nucleic acid as described herein.

Still further, aspects are drawn towards a cell comprising nucleic acids or vectors described herein.

Aspects are drawn to a cell producing the monoclonal antibody as described herein.

Aspects are directed to methods for treating cancer. In embodiments, the method can include administering a pharmaceutically effective amount of one or more EDIL3 inhibitors to a subject with cancer. In embodiments, the method for treating cancer can lead to the reversal of an immune excluded tumor microenvironment. In embodiments, the method for treating cancer can lead to the reversal of an immune dysfunctional tumor environment. In embodiments, the method for treating cancer can decrease the immune suppressive effects of cancer-associated fibroblasts (CAFs) in a subject with cancer. In some embodiments, the method of treating cancer can counteract the effects of EDIL3-dependent blocking of LFA-1 binding to endothelial cells. In some embodiments, the EDIL3 Inhibitor is an antibody or antigen-binding fragment or variant thereof that binds EDIL3 protein.

In some embodiments that method of treating cancer can include one or more additional therapies for treating cancer. In some embodiments the additional therapy is directed to angiogenesis and/or checkpoint blockade. In some embodiments, the method for treatment of cancer decreases the immune suppressive effects of cancer-associated fibroblasts (CAFs) in a subject. In some embodiments, the method of treating cancer can counteract the effects of EDIL3-dependent blocking of LFA-1 binding to ICAM-1. In some embodiments, the method of treating cancer can counteract the effects of EDIL3-dependent blocking of LFA-1 binding to endothelial cells. In some embodiments, the EDIL3 inhibitor is an antibody or antigen-binding fragment or variant thereof that specifically binds EDIL3 protein. In some embodiments, the method for treating cancer can include one or more additional therapies to treat cancer. In some embodiments, the additional therapy to treat cancer is a therapy that can modify angiogenesis. In some embodiments, the additional therapy to treat cancer is a therapy that can modify checkpoint blockade.

### BRIEF DESCRIPTION OF THE FIGURES

FIG. 1 shows humoral immune response by ipilimumab plus bevacizumab (Ipi-Bev) elicited to EDIL3 was associated with clinical outcomes in metastatic melanoma patients. Panel a, EDIL3 antibody fold change in pre-treatment vs pos-treatment plasma samples of 42 Ipi-Bev patients measured by ELISA. Each bar represents a patient, and the color of the bar indicates clinical assessment (CR, complete response (green); PR, partial response (green); SD, stable disease (blue); and PD, progressive disease (red)). EDIL3 antibody titer was considered significant when fold change was ≥1.5. Panel b, Frequency of EDIL3 antibody increases by clinical responses. Panel c, Immunoblot blot analysis of EDIL3 Ig expression in pre-treatment and post-treatment plasma samples of representative patients. Panel d, Kaplan-Meier survival curves of patients based on EDIL3 antibody fold change ≥1.5 or ≤1.5 (P=0.027). The median survival of the patients with EDIL3 antibody fold change <1.5 was 70 weeks (95% CI, 47-81), whereas that of patients with fold change ≥1.5 was not reached. Panel e, Percentage of patients with EDIL3 antibody fold change >1.5 in patient cohorts treated with Ipi-Bev (n=42), ipilimumab (n=34), PD-1 blockade (n=25), and Nivolumab-Ipilimumab (n=41).

FIG. 2 shows EDIL3 expression is associated with tumor T cell immune exclusion gene signatures by TIDE analysis. Panel a, Volcano plot of EDIL3, MFGE8 and CTNNB1 for dysfunction vs exclusion correlation across TCGA tumor types. Panel b, EDIL3, MFGE8 and CTNNB1 expression and predictive value of T cell exclusion for immunotherapy response in TCGA Skin cutaneous melanoma (SKCM) dataset.

FIG. 3 shows EDIL3 mediated T-cell exclusion is associated with TGFβ signaling, EMT, and angiogenesis signatures. Cancer associated fibroblast (CAF) FAP signature score correlated with EDIL3 and MFGE8 expression values in T cell exclusion as determined using the TIDE method (heat map data not shown).

FIG. 4 shows EDIL3 is abundantly expressed in CAFs and is upregulated by TGF-β1 inducing EMT. Panel a, Detection of secreted EDIL3 in condition medium from normal fibroblast (NF) and patients derived CAFs (P4-CAF and CAF2) by ELISA. Panels b-c, NF pretreated with or without LY2109761 (LY) followed by TGF-β1 treatment for 24 h. EDIL3 expression was examined by Panel (b) ELISA of conditioned medium and Panel (c) immunoblot blot analyses of whole cell lysates. Panel d, Quantitative RT-PCR analysis of EDIL3 silencing in CAFs and TGFβ1 mediated induction of EDIL3 and target genes Panel e, Transgelin (TAGLN) and Panel f, α-SMA (ACTA2) in control vs EDIL3 siRNA. *P<0.05, **P<0.01, ***P<0.001, ****P<0.0001. Means±SD of three independent experiments is shown.

FIG. 5 shows EDIL3 expression correlates with serum VEGF levels and angiogenesis signatures in TCGA SKCM and Checkmate 064 databases. Panel a, Correlation of pre-treatment circulating serum levels of EDIL3 versus VEGF-Ain Ipi-Bev treated melanoma patients (n=42). Panel b, EDIL3 expression is significantly associated with high angiogenic signatures in TCGA SKCM and Checkmate 064 datasets. P Panel c, EDIL3 promoted tube formation ability of patient-derived endothelial cells comparable to VEGF as assessed by Angiogenesis assay. Means±SD of three independent experiments is shown.

FIG. 6 shows EDIL3 blocks lymphocyte-endothelial adhesion and inhibits T cell migration. Panel a, Expression of ICAM-1 ligand LFA-1 in immune cells was screened via flow cytometry. Panels b-c, Confluent monolayers of patient-derived endothelial cells were left unstimulated or stimulated with TNF-α for 24 h followed by 45 min incubation with THP-1 cells or activated Leukocytes pre-treated with human recombinant EDIL3 or/and anti-LFA-1 antibody for 1 h, were allowed to adhere at 37° C. (b) EDIL3 inhibits adhesion of THP-1 cells to TNFα-activated endothelial cells in a dose dependent manner. Panel (c) EDIL3 blocks the lymphocyte-endothelial adhesion by interfering LFA-1 and ICAM-1 interaction. Panel d, EDIL3 silenced patient-derived endothelial cells were grown in transwells and incubated with T-cells in presence or absence of the chemoattractant IP-10 in the bottom chamber, migration across the endothelial monolayer was measured after 2-4 h. EDIL3 silencing in endothelial cells potentiated T-cell transwell trans-endothelial migration. Panel e, Patient derived endothelial cells were cultured on transwell and were incubated with untreated or EDIL3 pre-treated T cells in presence or absence of IP-10 in the bottom chamber, their migration across the endothelial monolayer was measured after 2-4 h. rEDIL3 partially blocked T-cell transwell trans-endothelial migration.

FIG. 7 shows (panel a) the addition of EDIL3 pretreated T-cells and rEDIL3 in media decreased the overall IP-10 induced migration (p<0.01) of T-cells, and that (panel b) EDIL3 pretreatment significantly (p<0.05) downregulated the transmigration of T-cells across the vascular networks.

FIG. 8 shows humoral response elicited to Ipi-Bev therapy in metastatic melanoma patients. Panel a, Frequencies of MFGE8 antibody by clinical response (CR, complete response; PR, partial responses; PD, progressive diseases; SD, stable disease). Panel b, Kaplan-Meier survival analysis of patients based on MFGE8 antibody fold change≥1.5 or ≤1.5 (P=0.61). Panel c, EDIL3 antibody titers in the pre-treatment and post-treatment plasma samples of patients with improved clinical outcomes.

FIG. 9 shows Ipi-Bev therapy altered IgG titers for EDIL3 and MFGE8 antibody response. Pre-treatment (Pre) and post-treatment (Post) Ig titers to Ipi-Bev therapy in patients for: Panel a, EDIL3 (n=42) and Panel b, MFGE8 (n=39). Pre-treatment and post-treatment Ig titers to Ipi-Bev therapy in patients grouped by clinical responses for: Panel c, EDIL3 (CR/PR (n=22), PD (n=13) and SD (n=7)) and Panel d, MFGE8 (CR/PR (n=6), PD (n=9) and SD (n=18)).

FIG. 10 shows longitudinal analysis of EDIL3 antibody response to Ipi-Bev treatment in metastatic melanoma patients. The optical density (OD) obtained by ELISA of EDIL3 antibody titers for representative clinical responses was plotted against time relative to initiation of Ipi-bev treatment in patients. Anti-EDIL3 response over time is shown for: Panel a P6; Panel b P13; Panel c P12; Panel d P21; Panel e P17; and Panel f P26. CR, complete response; PR, partial responses; PD, progressive diseases; SD, stable disease.

FIG. 11 shows TPM (transcripts per million) from RNA-sequencing. Panel a, EDIL3 overexpression in CAFs (cancer associated fibroblasts) vs NF (normal fibroblast) and its induction by TGFβ1. Panel b, MFGE8 overexpression in CAFs vs NF and unaltered by TGFβ1. Panels c-d, CAFs showing positive expression of surface markers FAP and LRRC15 under TGFβ1control.

FIG. 12 shows molecular validation data. Panel a, Relative expression of EDIL3 in endothelial cells (HDMEC, TEC (tumor endothelial cells) and HUVEC). Panel b, Validation of TNFα mediated activation of HUVEC and TECs via induction of ICAM1 expression. Panel c, EDI13 silencing in TEC using siRNA. Panel d, Representative images, and quantification of binding assay showing activated THP-1 cells binds to EDIL3 with higher affinity vs ICAM1. Panel e, Representative images for adhesion assay from FIG. 6 Panel c.

FIG. 13 shows (panel a) western blot and (panels b and c) graphs indicating that combination therapy patients had antibody responses to EDIL3 as a function of treatment.

FIG. 14 shows MFGE8 related clinical outcomes. Panel A shows a graph of MFGE8 Ig frequency by outcome. Panel B shows a graph of overall survival percentage.

FIG. 15 shows EDIL3 and MFG-E8 in melanoma and melanoma cell lines. Panel A shows 064 data. Panel B shows melanoma cell line data. Panel C shows a Western blot.

FIG. 16 shows EDIL3 expression. Panels A and B show graphs of EDIL3 and Panel C shows a Western blot.

FIG. 17 shows CAF expression levels.

FIG. 18 shows relative mRNA expression for EDIL3 (panel A), ACTA2 (panel B), and TGLN (panel C).

FIG. 19 shows relative mRNA expression data.

FIG. 20 shows binding activities of PR305734, PR305667, PR305633, PR305618, PR305754-p, PR305764, PR305689, PR305684, PR305629 and PR305623 antibodies to human EDIL3 protein, Cyno EDIL3 protein and mouse EDIL3 protein. Panel A shows test results of the binding of antibodies to EDIL3 at the protein level. Panels B-D show binding of antibodies PR305618, PR305623, PR305629, PR305633 to human EDIL3 protein (Panel B), Cyno EDIL3 protein (Panel C) and mouse EDIL3 protein (Panel D). Panels E-G show binding of antibodies PR305667, PR305684, PR305689 to human EDIL3 protein (Panel E), Cyno EDIL3 protein (Panel F) and mouse EDIL3 protein (Panel G). Panels H-J show binding of antibodies PR305734, PR305764, and PR305754-p to human EDIL3 protein (Panel H), Cyno EDIL3 protein (Panel I) and mouse EDIL3 protein (Panel J).

FIG. 21 shows blocking activities of antibodies PR305734, PR305667, PR305633, PR305618, PR305754-p, PR305764, PR305689, PR305684, PR305629 and PR305623. Panel A shows a summary of blocking activities for purified Anti-EDIL3 antibodies. IC50 values are shown in the table. Panels B-C show PR305618, PR305623, PR305629, PR305633 blocking activity to human EDIL3 binding with ligand LFA-1 protein (Panel B) and αvβ3 protein by ELISA (Panel C). Panels D-E show PR305667, PR305684, PR305689 blocking activity to human EDIL3 binding with ligand LFA-1 protein (Panel D) and αvβ3 protein by ELISA (Panel E). Panels F-G show PR305734, PR305754-p, PR305764 blocking ability to the binding of human EDIL3 with ligand LFA-1 protein (Panel F) and αvβ3 protein (Panel G) with ELISA.

FIG. 22 shows amino acid sequences and nucleic acid sequences of embodiments of the invention.

FIG. 23 shows EDIL3 mAbs binds specifically to human recombinant EDIL3. EDIL3~65 kDa predicted band in reducing conditions.

FIG. 24 shows determination of kinetics and thermodynamics constants for EDIL3 mAbs by surface plasma resonance.

FIG. 25 shows the blocking activity of anti-EDIL3 mAbs.

FIG. 26 shows SPR-based kinetics characterization for anti-EDIL3 mAbs. The anti-EDIL3 mAbs show nM or sub-nM affinities for EDIL3 and slow off-rate kinetics (~1×10⁻⁵⁻⁶ min⁻¹).

FIG. 27 shows epitope binding analysis of anti-EDIL3 mAbs. The anti-EDIL3 mAbs bind overlapping yet distinct epitopes on EDIL3 that is dependent on antibody presentation (i.e. solution vs. surface presentation). None of these antibodies interact with EDIL3 in the presence of themselves (i.e. show biparatopic activity) or interact with EDIL3 in the presence of the negative control.

### DETAILED DESCRIPTION

Aspects described herein can lead to the reversal of immune excluded or immune dysfunctional tumor microenvironment. In embodiments, this approach identifies a specific target, EDIL3, that can facilitate immune cell traffic in the tumor microenvironment and reverses immune suppressive effects of cancer-associated fibroblasts. In embodiments, EDIL3 can be targeted in conjugation with anti-angiogenic treatment and/or immune checkpoint blockade therapy. Without wishing to be bound by theory, we developed and validated monoclonal antibodies against EDIL3 for reversing immune exclusion.

The present disclosure provides antibodies or antigen-binding fragments or variants thereof that specifically binds epidermal growth factor-like repeats and discoidin I-like domains 3 (EDIL3) protein.

The present disclosure further provides therapeutic antibodies or antigen-binding fragments or variants thereof that specifically binds epidermal growth factor-like repeats and discoidin I-like domains 3 (EDIL3) protein.

Still further, the present disclosure provides a method of treating cancer by administering to a subject in need thereof an effective amount of the anti-EDIL3 antibody or anti-EDIL3 therapeutic antibody.

Also, the present disclosure provides a method of decreasing immune suppressive effects of cancer-associated fibroblasts in a subject by administering to the subject an effective amount of the anti-EDIL3 antibody or anti-EDIL3 therapeutic antibody.

Further, the present disclosure is drawn to a nucleic acid encoding an anti-EDIL3 mAb.

The present disclosure also provides an antibody of the present disclosure for use in therapy. More particularly, the present disclosure provides an antibody of the present disclosure for use in treatment of cancer. Further, the present disclosure provides the use of an antibody of the present disclosure in the manufacture of a medicament for the treatment of cancer.

Disclosed herein are isolated, recombinant monoclonal antibodies that specifically bind to EDIL3. In some embodiments, the recombinant monoclonal antibody can bind to the recombinant human EDIL3-FC or EDIL3-His.

Herein, “specifically binds” or “immunoreacts with” can refer to the antibody reacting with one or more antigenic determinants of EDIL3 and does not react with other polypeptides.

The term “isolated” as used herein, such as with respect to cells, proteins or polypeptides, or nucleic acids (e.g., DNA or RNA), can refer to those cells, proteins or polypeptides, or nucleic acids that are purified to some degree from endogenous materials The term “isolated” can also refer to a nucleic acid or peptide that is substantially free of cellular material, viral material, or culture medium when produced by recombinant DNA techniques, or chemical precursors or other chemicals when chemically synthesized. For example, an “isolated nucleic acid” can include nucleic acid fragments which are not naturally occurring as fragments and would not be found in the natural state. “Isolated” can also refer to cells or polypeptides which are isolated from other cellular proteins or tissues. Isolated polypeptides can include both purified and recombinant polypeptides.

The term “recombinant” as it pertains to polypeptides (such as antibodies) or polynucleotides can refer to a form of the polypeptide or polynucleotide that does not exist naturally, a non-limiting example of which can be created by combining polynucleotides or polypeptides that would not normally occur together.

The EDIL3 antibodies described herein bind to EDIL3. In one embodiment, the EDIL3 antibodies have high affinity and high specificity for EDIL3. In some embodiments, EDIL3 antibodies can block EDIL3 binding to LFA-1. In some embodiments, blocking of EDIL3 binding to LFA-1 can allow binding of immune cell to endothelia cells and cross over of immune cells into the TME. Without wishing to be bound by theory, the antibodies described herein can counteract the effects of EDIL3-dependent blocking LFA-1 binding, and therefore can increase permissiveness of lymphocytes to traffic across the endothelium of blood vessels into the tumor microenvironment.

Herein, an “antibody” can refer to an immunoglobulin molecule comprising two heavy chains (HCs) and two light chains (LCs) interconnected by disulfide bonds. The amino terminal portion of each LC and HC includes a variable region of about 100-120 amino acids primarily responsible for antigen recognition via the complementary determining region (CDRs) contained therein. The CDRs are interspersed with regions that are well-known and generally conserved among and between species (e.g., mouse and human), which are termed framework regions (FRs). The amino acids comprising the CDRs and the framework regions, respectively, can be readily identified for a heavy or light chain variable region by one of ordinary skill in the art, since they have been previously defined

In embodiments, the CDRs are interspersed with FRs. Antibodies disclosed herein have four FRs, termed FR1, FR2, FR3, and FR4. In embodiments, the FRs are human FRs (e.g., Antibody Engineering: Methods and Protocols (Damien Nevoltris and Patrick Chames eds., 3d ed. 2018)).

The three CDRs of the light chain (LC) are referred to as “LCDR1, LCDR2, and LCDR3,” and the three CDRs of the heavy chain (HC) are referred to as “HCDR1, HCDR2, and HCDR3.” The functional ability of an antibody to bind a particular antigen is largely determined by the six CDRs. Assignment of amino acids to CDR domains within the LCVR and HCVR regions of the antibodies of the present disclosure is based on the Kabat numbering conventions.

The constant region of the antibody defines the isotype of an antibody. The antibodies of the present disclosure include IgG. IgG antibodies can be further divided into subclasses, e.g., IgG1, IgG2, IgG3, IgG4. In a particular embodiment, the antibodies of the present disclosure are IgG1. The carboxy-terminal portion of each HC defines a constant region primarily responsible for effector function. In a particular embodiment, the antibodies of the present disclosure have one or more modifications in the constant region of each HC that reduces effector function.

EDIL3 binds to LFA-1 integrin and thereby prevents intracellular adhesion molecule-1 (ICAM-1) binding, thus preventing leukocytes from trafficking across the endothelium. Accordingly, EDIL3 contributes to carcinogenesis by reducing apoptosis in cancer cells and promotes tumor vascularization. Therefore, EDIL3 is a target for immunotherapy. As used herein, the term “EDIL3 antibody” can refer to an antibody that binds EDIL3 and, without wishing to be bound by theory, disrupts EDIL3 from binding to LFA-1 integrin. Antibodies within the scope of the antibody are the disclosed antibodies and functional equivalents thereto. Functional equivalent antibodies comprise different specific amino acid residues but maintain binding activity to EDIL3. Functional equivalent antibodies would differ insubstantially in their abilities to disrupt EDIL3 from binding to LFA-1 integrin, for example, and therefore have a therapeutic effect.

The nucleic acid and amino acid sequences of the monoclonal EDIL3 antibodies are provided below:

TABLE 1A
Amino acid sequences of heavy chain variable region (HCVR).

| Antibody | |
|---|---|
| ID | Sequence |
| PR305618 | EVQLLESGGGLVQPGGSLRLSCAASGFTESSYAMSWVRQAPGKGLEWVSA |
| VH | ISDSGGSTYYADSVKGRETISRDNSKNTLYLQMNSLRAEDTAVYYCAQEG |
| | LITFGGVIVIGYEDYWGQGTLVTVSS |
| PR305623 | EVQLVESGGGLVQPGGSLRLSCAASGFTESSYWMSWVRQAPGKGLEWVAN |
| VH | IKQDGSQKYYVDSVKGRFTISRDNAKNSLYLQMNSLRAEDTAVYYCTRRG |
| | NEFFDNWGQGTLVTVSS |
| PR305629 | QVQLQESGPGLVKPSETLSLTCTVSGGSISNHYWSWIRQPPGKGLEWIGY |
| VH | IYYSGSTNYNPSLKSRVTISVDTSKNQFSLKLSSVTAADTAVYYCARGFA |
| | YWGQGTLVTVSS |
| PR305633 | EVQLLESGGGLGQPGGSLRLSCAASGFTFSSYAMNWVRQAPGKGLEWVSA |
| VH | ISGSGDSTYSTDSVKGRFTISRDNSKNTLYLQMNSLRAEDTAVYYCAKEY |
| | YDILTGYWQWYFDLWGQGTLVTVSS |
| PR305667 | QVQLVESGGGVVQPGRSLRLSCAASGFTESSYGMHWVRQAPGKGLEWVAV |
| VH | MWYDGSDRYSADSVKGRFTISRDNSKNTLYLQMNSLRAEDTAVYYCARGY |
| | DILTGPDHFDYWGQGTLVTVSS |
| PR305684 | EVQLLESGGGLVQPGGSLRLSCAASGFTESSYDMNWVRQAPGKGPVWVST |
| VH | ISGSGSHTYYADSVRGRFTISRDNSKNTLYLQMNSLRAEDTAVYYCAKEG |
| | GATAFDIWGQGTMVTVSS |
| PR305689 | QVQLVESGGGVVQPGRSLRLSCAASGFTESTYGMHWVRQAPGKGLEWVAL |
| VH | IWYDGINKYYADSVKGRFTISRDNSKNTLYLQMNSLRAEDTAVYECARPY |
| | YDILTGYFDYWGQGTLVTVSS |
| PR305734 | QVQLVESGGGVVQPGRSLRLSCAASGFTESSYGMHWVRQAPGKGLEWVAV |
| VH | IWYDGTNKYYADSVKGRETISRDNSKNTLYLQVNSLRAEDTAVYYCARDP |
| | SLWFGEFPHYYGMDVWGQGTTVTVSS |
| PR305754 | QVQLQQWGAGLLKPSETLSLTCAVYGGSFSGYYWSWIRQPPGKGLEWIGE |
| VH | IQHSGSTNYKPSLKSRVTISVDTSKNQESLKLSSVTAADTAVYYCAELTG |
| | DSLLFEYWGQGTLVTVSS |
| PR305764 | QVQLVESGGGVVQPGRSLRLSCAASGFTESSYGMHWVRQAPGKGLEWVAV |
| VH | IWYDGSNKYYADSVKGRFTISRDTSKNTLYLQMNSLRAEDTAVYYCARDS |
| | ASDYFDYWGQGTLVTVSS |
| PR305754-P | QVQLQQWGAGLLKPSETLSLTCAVYGGSFSGYYWSWIRQPPGKGLEWIGE |
| VH | INHSGSTNYKPSLKSRVTISVDTSKNQFSLKLSSVTAADTAVYYCAELTG |
| | DSLLFEYWGQGTLVTVSS |

TABLE 1B
Amino acid sequences of light chain variable region (LCVR).

| Antibody | |
|---|---|
| ID | Sequence |
| PR305618 | DIQMTQSPSSLSASVGDRVTITCQASQDISNYLNWYQQKPGKAPKLLIY |
| VL | DASNLETGVPSRFSGSGSGIDETFTISSLQPEDIATYYCQQYDNLPITE |
| | GQGTRLEIK |
| PR305623 | EIVLTQSPATLSLSPGERATLSCRASQYVSSYLAWYHQKPGQAPRLLIY |
| VL | DASNRATGIPARFSGSGSGTDFTLTISSLEPEDFAVYYCQQRNNWPPTE |
| | GQGTKVEIK |
| PR305629 | DIQMTQSPSSLSASVGDRVTITCRASQGITNYLAWYQQKPGKVPKLLIY |
| VL | AASTLQSGVPSRFSGSGSGIDETLTINSLQPEDVATYYCQKYNSAPWTF |
| | GQGTKVEIK |
| PR305633 | EIVMTLSPATLSVSPGERATLSCRASQSINSNLAWYQQKPGQAPRLLIY |
| VL | GASTRATGIPARESGSGSGTEFTLTISSLQSEDFAVYYCQQYNNWPLTE |
| | GGGTKVEIK |
| PR305667 | DIQMTQSPSSLSASVGDRVTITCRASQSISSYLNWYQQKPGKAPKLLIY |
| VL | AASSLQSGVPSRESGSGSGTDETLTISSLQPEDFATYYCQQSYSTPLIT |
| | GGGTKVEIK |
| PR305684 | DIQLTQSPSFLSASVGDRVTITCRASQGISSYLAWYQQKPGKAPKLLIY |
| VL | VASTLQSGVPSRFSGSGSGTEFTLTISSLQPEDFATYYCQQLNNYPTFG |
| | GGTKVEIK |
| PR305689 | EIVLTQSPGTLSLSPGERATLSCRASQSDSSSYLAWYQQKPGQAPRLLI |
| VL | YGTSSRATGISDRESGSGSGTDFTLTISRLEPEDFAVYYCQQYGSSPLT |
| | FGGGIKVEIK |
| PR305734 | DIQMTQSPSSLSASVGDRVTITCQASQDISNYLNWYQQKPGKAPKLLIY |
| VL | DASNLETGVPSRFSGSGSGTDETFTISSLQPEDIATYYCQQYDNLPLTE |
| | GGGTKVEIK |
| PR305754 | EIVLTQSPATLSLSPGERATLSCRASQSVSSYLAWYQQKPGQAPRLLIY |
| VL | DTSNRATGIPARFSGSGSGTDETLTVSSLEPEDEAVYYCQQRSNWPITE |
| | GQGTRLEIK |
| PR305764 | EIVMTQSPATLSVSPGERATLSCRASQSVSSNLAWYQQKPGQAPRLLIY |
| VL | GASTRATGIPARFSGSGSGTEFTLTISSLQSEDFAVYYCQQYSDWPTFG |
| | GGTKVEIR |
| PR305754-P | EIVLTQSPATLSLSPGERATLSCRASQSVSSYLAWYQQKPGQAPRLLIY |
| VL | DTSNRATGIPARFSGSGSGTDFTLTVSSLEPEDFAVYYCQQRSNWPITE |
| | GQGTRLEIK |

TABLE 2A
nucleic acid sequences of heavy chain variable
region (HCVR).

| Antibody | |
|---|---|
| ID | Sequence |
| PR305618 | GAGGTGCAGCTGTTGGAGTCTGGGGGAGGCTTGGTACAGCCTGGGGGGT |
| VH | CCCTGAGACTCTCCTGTGCAGCCTCTGGATTCACCTTTAGCAGCTATGC |
| | CATGAGCTGGGTCCGCCAGGCTCCAGGGAAGGGGCTGGAGTGGGTCTCA |
| | GCTATTAGTGATAGTGGTGGTAGCACATACTACGCAGACTCCGTGAAGG |
| | GCCGGTTCACCATCTCCAGAGACAATTCCAAGAACACGCTGTATCTGCA |
| | AATGAACAGCCTGAGAGCCGAGGACACGGCCGTATATTACTGTGCGCAG |
| | GAGGGTTTGATTACGTTTGGGGGAGTTATCGTTATAGGCTACTTTGACT |
| | ACTGGGGCCAGGGAACCCTGGTCACCGTCTCCTCA |
| PR305623 | GAGGTGCAGCTGGTGGAGTCTGGGGGAGGCTTGGTCCAGCCTGGGGGGT |
| VH | CCCTGAGACTCTCCTGTGCAGCCTCTGGATTCACCTTTAGTAGCTATTG |
| | GATGAGCTGGGTCCGCCAGGCTCCAGGGAAGGGGCTGGAATGGGTGGCC |
| | AATATAAAGCAAGATGGAAGTCAGAAATACTATGTGGACTCTGTGAAGG |
| | GCCGATTCACCATCTCCAGAGACAACGCCAAGAACTCACTGTATCTGCA |
| | AATGAACAGCCTGAGAGCCGAGGACACGGCTGTGTATTACTGTACGAGA |
| | CGTGGGAACTTCTTCTTTGACAATTGGGGCCAGGGAACCCTGGTCACCG |
| | TCTCCTCA |
| PR305629 | CAGGTGCAGCTGCAGGAGTCGGGCCCAGGACTGGTGAAGCCTTCGGAGA |
| VH | CCCTGTCCCTCACTTGCACTGTCTCTGGTGGCTCCATCAGTAATCACTA |
| | CTGGAGCTGGATTCGGCAGCCCCCAGGGAAGGGACTGGAGTGGATTGGG |
| | TATATCTATTACAGTGGGAGCACCAACTACAACCCCTCCCTCAAGAGTC |
| | GAGTCACCATATCAGTAGACACGTCCAAGAACCAGTTCTCCCTGAAGCT |
| | GAGCTCTGTGACCGCTGCGGACACGGCCGTGTATTACTGTGCGAGGGGG |
| | TTTGCTTACTGGGGCCAAGGCACTCTGGTCACTGTCTCTTCA |
| PR305633 | GAGGTGCAGCTGTTGGAATCTGGGGGAGGCTTGGGACAGCCTGGGGGGT |
| VH | CCCTGAGACTCTCCTGTGCAGCCTCTGGATTCACCTTTAGCAGCTATGC |
| | CATGAACTGGGTCCGCCAGGCTCCAGGGAAGGGGCTGGAGTGGGTCTCA |
| | GCTATCAGTGGCAGTGGTGATAGCACATACTCCACAGACTCCGTGAAGG |
| | GCCGGTTCACCATCTCCAGAGACAATTCCAAGAACACGCTGTATCTGCA |
| | AATGAACAGCCTGAGAGCCGAGGACACGGCCGTTTATTACTGTGCGAAA |
| | GAGTATTACGATATTTTGACTGGTTATTGGGACTGGTACTTCGATCTCT |
| | GGGGCCAAGGCACCCTGGTCACTGTCTCCTCA |
| PR305667 | CAGGTGCAGCTGGTGGAGTCTGGGGGAGGCGTGGTCCAGCCTGGGAGGT |
| VH | CCCTGAGACTCTCCTGTGCAGCGTCTGGATTCACCTTCAGTAGCTATGG |
| | CATGCATTGGGTCCGCCAGGCTCCAGGCAAGGGGCTGGAGTGGGTGGCA |
| | GTTATGTGGTATGATGGAAGTGATAGATACTCTGCAGACTCCGTGAAGG |
| | GCCGATTCACCATCTCCAGAGACAATTCCAAGAACACGCTGTATTTGCA |
| | AATGAACAGCCTGAGAGCCGAGGACACGGCTGTGTATTACTGTGCGAGG |
| | GGGTACGATATTTTGACTGGTCCCGACCACTTTGACTACTGGGGCCAGG |
| | GAACCCTGGTCACCGTCTCCTCA |
| PR305684 | GAAGTACAGTTGTTGGAGTCTGGGGGAGGCTTGGTACAGCCTGGGGGGT |
| VH | CCCTGAGACTCTCCTGTGCAGCCTCTGGATTCACCTTTAGCAGCTATGA |
| | CATGAACTGGGTCCGCCAGGCTCCAGGGAAGGGGCCGGTGTGGGTCTCA |
| | ACTATTAGTGGTAGTGGTAGTCACACATACTACGCAGACTCCGTGAGGG |
| | GCCGGTTCACCATCTCCAGAGACAACTCCAAGAACACGCTGTATCTGCA |
| | AATGAACAGCCTGAGAGCCGAGGACACGGCCGTATATTACTGTGCGAAA |
| | GAGGGGGGAGCTACTGCTTTTGATATCTGGGGCCAAGGGACAATGGTCA |
| | CCGTCTCTTCA |
| PR305689 | CAGGTGCAGCTGGTGGAGTCTGGGGGAGGCGTGGTCCAGCCTGGGAGGT |
| VH | CCCTGAGACTCTCCTGTGCAGCGTCTGGATTCACCTTCAGTACCTATGG |
| | CATGCACTGGGTCCGCCAGGCTCCAGGCAAGGGGCTGGAGTGGGTGGCA |
| | CTTATATGGTATGATGGAATTAATAAATACTATGCGGACTCCGTGAAGG |
| | GCCGATTCACCATCTCCAGAGACAATTCCAAGAACACGCTGTATCTGCA |
| | AATGAACAGCCTGAGAGCCGAGGACACGGCTGTGTATTTCTGTGCGAGA |
| | CCCTATTACGATATTTTGACTGGTTATTTTGACTACTGGGGCCAGGGAA |
| | CCCTGGTCACCGTCTCCTCA |
| PR305734 | CAGGTGCAGCTGGTGGAGTCTGGGGGAGGCGTGGTCCAGCCTGGGAGGT |
| VH | CCCTGAGACTCTCCTGTGCAGCGTCTGGATTCACCTTCAGTAGCTATGG |
| | CATGCACTGGGTCCGCCAGGCTCCAGGCAAGGGGCTGGAGTGGGTGGCA |
| | GTTATATGGTATGATGGAACTAATAAATACTATGCAGACTCCGTGAAGG |
| | GCCGATTCACCATCTCCAGAGACAATTCCAAGAACACGCTGTATCTGCA |
| | AGTGAACAGCCTGAGAGCCGAGGACACGGCTGTGTATTACTGTGCGAGA |
| | GATCCCTCCTTATGGTTCGGGGAGTTCCCTCATTACTACGGTATGGACG |
| | TCTGGGGCCAAGGGACCACGGTCACCGTCTCCTCA |
| PR305754 | CAGGTGCAGCTACAGCAGTGGGGCGCAGGACTGTTGAAGCCTTCGGAGA |
| VH | CCCTGTCCCTCACCTGCGCTGTCTATGGTGGGTCCTTCAGTGGTTACTA |
| | CTGGAGCTGGATACGCCAGCCCCCAGGGAAGGGGCTGGAGTGGATTGGG |
| | GAAATCCAACATAGTGGAAGCACCAACTACAAACCGTCCCTCAAGAGTC |
| | GAGTCACCATATCAGTAGACACGTCCAAGAACCAGTTCTCCCTGAAGCT |
| | GAGCTCTGTGACCGCCGCGGACACGGCTGTGTATTACTGTGCGGAGCTA |
| | ACTGGGGATTCCCTTTTGTTTGAGTACTGGGGCCAGGGAACCCTGGTCA |
| | CCGTCTCCTCA |
| PR305764 | CAGGTGCAGCTGGTGGAGTCTGGGGGAGGCGTGGTCCAGCCTGGGAGGT |
| VH | CCCTGAGACTCTCCTGTGCAGCGTCTGGATTCACCTTCAGTAGCTATGG |
| | CATGCACTGGGTCCGCCAGGCTCCAGGCAAGGGGCTGGAGTGGGTGGCA |
| | GTTATATGGTATGATGGAAGTAATAAATACTATGCAGACTCCGTGAAGG |
| | GCCGATTCACCATCTCCAGAGACACTTCCAAGAACACACTGTATCTGCA |
| | AATGAACAGCCTGAGAGCCGAGGACACGGCTGTGTATTACTGTGCGAGA |
| | GATAGCGCCTCCGACTACTTTGACTACTGGGGCCAGGGAACCCTGGTCA |
| | CCGTCTCCTCA |
| PR305754-P | CAGGTGCAGCTACAGCAGTGGGGCGCAGGACTGTTGAAGCCTTCGGAGA |
| VH | CCCTGTCCCTCACCTGCGCTGTCTATGGTGGGTCCTTCAGTGGTTACTA |
| | CTGGAGCTGGATACGCCAGCCCCCAGGGAAGGGGCTGGAGTGGATTGGG |
| | GAAATCAATCATAGTGGAAGCACCAACTACAAACCGTCCCTCAAGAGTC |
| | GAGTCACCATATCAGTAGACACGTCCAAGAACCAGTTCTCCCTGAAGCT |
| | GAGCTCTGTGACCGCCGCGGACACGGCTGTGTATTACTGTGCGGAGCTA |
| | ACTGGGGATTCCCTTTTGTTTGAGTACTGGGGCCAGGGAACCCTGGTCA |
| | CCGTCTCCTCA |

TABLE 2B
nucleic acid sequences of light chain variable region (LCVR).

| Antibody | |
|---|---|
| ID | Sequence |
| PR305618 | GACATCCAGATGACCCAGTCTCCATCCTCCCTGTCTGCATCTGTAGGAGA |
| VL | CAGAGTCACCATCACTTGCCAGGCGAGTCAGGACATTAGCAACTATTTAA |
| | ATTGGTATCAGCAGAAACCAGGGAAAGCCCCTAAGCTCCTGATCTACGAT |
| | GCATCCAATTTGGAAACAGGGGTCCCATCAAGGTTCAGTGGAAGTGGATC |
| | TGGGACAGATTTTACTTTCACCATCAGCAGCCTGCAGCCTGAAGATATTG |
| | CAACATATTACTGTCAACAGTATGATAATCTCCCGATCACCTTCGGCCAA |
| | GGGACACGACTGGAGATTAAA |
| PR305623 | GAAATTGTGTTGACACAGTCTCCAGCCACCCTGTCTTTGTCTCCAGGGGA |
| VL | AAGAGCCACCCTCTCCTGCAGGGCCAGTCAGTATGTTAGCAGCTACTTAG |
| | CCTGGTACCACCAGAAACCTGGCCAGGCTCCCAGGCTCCTCATCTACGAT |
| | GCATCCAACAGGGCCACTGGCATCCCAGCCAGGTTCAGTGGCAGTGGGTC |
| | TGGGACAGACTTCACTCTCACCATCAGCAGCCTAGAGCCTGAAGATTTTG |
| | CAGTTTATTACTGTCAGCAGCGTAACAACTGGCCTCCGACGTTCGGCCAA |
| | GGGACCAAGGTGGAAATCAAA |
| PR305629 | GACATCCAGATGACCCAGTCTCCATCCTCCCTGTCTGCATCTGTAGGAGA |
| VL | CAGAGTCACCATCACTTGCCGGGCGAGTCAGGGCATTACCAATTATTTAG |
| | CCTGGTATCAGCAGAAACCAGGGAAAGTTCCTAAGCTCCTGATCTATGCT |
| | GCATCCACTTTGCAATCAGGGGTCCCATCTCGGTTCAGTGGCAGTGGATC |
| | TGGGACAGATTTCACTCTCACCATCAACAGCCTGCAGCCTGAAGATGTTG |
| | CAACTTATTACTGTCAAAAGTATAACAGTGCCCCGTGGACGTTCGGCCAA |
| | GGGACCAAGGTGGAAATCAAA |
| PR305633 | GAAATAGTTGATGACGCTGTCTCCAGCCACCCTGTCTGTGTCTCCAGGGGA |
| VL | AAGAGCCACCCTCTCCTGCAGGGCCAGTCAGAGTATTAACAGCAACTTAG |
| | CCTGGTACCAGCAGAAACCTGGCCAGGCTCCCAGGCTCCTCATCTATGGT |
| | GCATCCACCAGGGCCACTGGTATCCCAGCCAGGTTCAGTGGCAGTGGGTC |
| | TGGGACAGAGTTCACTCTCACCATCAGCAGCCTGCAGTCTGAAGATTTTG |
| | CAGTTTATTACTGTCAGCAGTATAATAACTGGCCGCTCACTTTCGGCGGA |
| | GGGACCAAGGTGGAGATCAAA |
| PR305667 | GACATCCAGATGACCCAGTCTCCATCCTCCCTGTCTGCATCTGTAGGAGA |
| VL | CAGAGTCACCATCACTTGCCGGGCAAGTCAGAGCATTAGCAGTTATTTAA |
| | ATTGGTATCAGCAGAAACCAGGGAAAGCCCCTAAGCTCCTGATCTATGCT |
| | GCATCCAGTTTGCAAAGTGGGGTCCCATCGAGGTTCAGTGGCAGTGGATC |
| | TGGGACAGATTTCACTCTCACCATCAGCAGTCTGCAACCTGAAGATTTTG |
| | CAACTTACTATTGTCAACAGAGTTACAGTACCCCGCTCACTTTCGGGGGA |
| | GGGACCAAGGTGGAGATCAAA |
| PR305684 | GACATCCAGTTGACCCAGTCTCCATCCTTCCTGTCTGCATCTGTAGGAGA |
| VL | CAGAGTCACCATCACTTGCCGGGCCAGTCAGGGCATTAGCAGTTATTTAG |
| | CCTGGTATCAGCAAAAACCAGGGAAAGCCCCTAAGCTCCTGATCTATGTT |
| | GCATCCACTTTGCAAAGTGGGGTCCCATCAAGGTTCAGCGGCAGTGGATC |
| | TGGGACAGAATTCACTCTCACAATCAGCAGCCTGCAGCCTGAAGATTTTG |
| | CAACTTATTACTGTCAACAGCTTAATAATTACCCCACTTTCGGCGGAGGG |
| | ACCAAGGTGGAGATCAAA |
| PR305689 | GAAATTGTGTTGACGCAGTCTCCAGGCACCCTGTCTTTGTCTCCAGGGGA |
| VL | AAGAGCCACCCTCTCCTGCAGGGCCAGTCAGAGTGATAGCAGCAGCTACT |
| | TAGCCTGGTATCAGCAGAAACCTGGCCAGGCTCCCAGGCTCCTCATATAT |
| | GGTACATCCAGTAGGGCCACTGGCATCTCAGACAGGTTCAGTGGCAGTGG |
| | GTCTGGGACAGACTTCACTCTCACCATCAGCAGACTGGAGCCTGAAGATT |
| | TTGCAGTGTATTACTGTCAGCAGTATGGTAGCTCACCGCTCACTTTCGGC |
| | GGAGGGACCAAGGTGGAGATCAAA |
| PR305734 | GACATCCAGATGACCCAGTCTCCATCCTCCCTGTCTGCATCTGTAGGAGA |
| VL | CAGAGTCACCATCACTTGCCAGGCGAGTCAGGACATTAGCAATTATTTAA |
| | ATTGGTATCAGCAGAAACCAGGGAAAGCCCCTAAGCTCCTGATCTACGAT |
| | GCATCCAATTTGGAAACAGGGGTCCCATCAAGGTTCAGTGGAAGTGGATC |
| | TGGGACAGATTTTACTTTCACCATCAGCAGCCTGCAGCCTGAAGATATTG |
| | CAACATATTACTGTCAACAGTATGATAATCTCCCGCTCACTTTCGGCGGA |
| | GGGACCAAGGTGGAGATCAAA |
| PR305754 | GAAATTGTGTTGACACAGTCTCCAGCCACCCTGTCTTTGTCTCCAGGGGA |
| VL | AAGAGCCACCCTCTCCTGCAGGGCCAGTCAGAGTGTTAGCAGCTACTTAG |
| | CCTGGTACCAACAGAAACCTGGCCAGGCTCCCAGGCTCCTCATCTATGAT |
| | ACATCCAACAGGGCCACTGGCATCCCAGCCAGGTTCAGTGGCAGTGGGTC |
| | TGGGACAGACTTCACTCTCACCGTCAGCAGCCTAGAGCCTGAAGATTTTG |
| | CAGTTTATTACTGTCAGCAGCGTAGCAACTGGCCGATCACCTTCGGCCAA |
| | GGGACACGACTGGAGATTAAA |
| PR305764 | GAAATAGTGATGACGCAGTCTCCAGCCACCCTGTCTGTGTCTCCAGGGGA |
| VL | AAGAGCCACCCTCTCCTGCAGGGCCAGTCAGAGTGTTAGCAGCAACTTAG |
| | CCTGGTACCAGCAGAAACCTGGCCAGGCTCCCAGGCTCCTCATCTATGGT |
| | GCATCCACCAGGGCCACTGGTATCCCAGCCAGATTCAGTGGCAGTGGGTC |
| | TGGGACAGAGTTCACTCTCACCATCAGCAGCCTGCAGTCTGAAGATTTTG |
| | CAGTTTATTACTGTCAGCAGTATAGTGACTGGCCCACTTTCGGCGGAGGG |
| | ACCAAGGTGGAGATCAGA |
| PR305754-P | GAAATTGTGTTGACACAGTCTCCAGCCACCCTGTCTTTGTCTCCAGGGGA |
| VL | AAGAGCCACCCTCTCCTGCAGGGCCAGTCAGAGTGTTAGCAGCTACTTAG |
| | CCTGGTACCAACAGAAACCTGGCCAGGCTCCCAGGCTCCTCATCTATGAT |
| | ACATCCAACAGGGCCACTGGCATCCCAGCCAGGTTCAGTGGCAGTGGGTC |
| | TGGGACAGACTTCACTCTCACCGTCAGCAGCCTAGAGCCTGAAGATTTTG |
| | CAGTTTATTACTGTCAGCAGCGTAGCAACTGGCCGATCACCTTCGGCCAA |
| | GGGACACGACTGGAGATTAAA |

TABLE 3A
Amino acid sequences of heavy chain (HC) complementarity
determining regions (CDRs).

| Antibody | CDR1 | CDR2 | CDR3 |
|---|---|---|---|
| PR305618 HC | SYAMS | AISDSGGSTYYADSVKG | EGLITFGGVIVIGYEDY |
| PR305623 HC | SYWMS | NIKQDGSQKYYVDSVKG | RGNEFFDN |
| PR305629 HC | NHYWS | YIYYSGSTNYNPSLKS | GFAY |
| PR305633 HC | SYAMN | AISGSGDSTYSTDSVKG | EYYDILTGYWDWYFDL |
| PR305667 HC | SYGMH | VMWYDGSDRYSADSVKG | GYDILTGPDHFDY |
| PR305684 HC | SYDMN | TISGSGSHTYYADSVRG | EGGATARDI |
| PR305689 HC | TYGMH | LIWYDGINKYYADSVKG | PYYDILTGYFDY |
| PR305734 HC | SYGMH | VIWYDGINKYYADSVKG | DPSLWFGEFPHYYGMDV |
| PR305754 HC | GYYWS | EIQHSGSTNYKPSLKS | LTGDSLLFEY |
| PR305764 HC | SYGMH | VIWYDGSNKYYADSVKG | DSASDYEDY |
| PR305754-P HC | GYYWS | EINHSGSTNYKPSLKS | LTGDSLLFEY |

TABLE 3B
Amino acid sequences of light chain (LC)
complementarity determining regions (CDRs).

| Antibody | CDR1 | CDR2 | CDR3 |
|---|---|---|---|
| PR305618 LC | QASQDISNYLN | DASNLET | QQYDNLPIT |
| PR305623 LC | RASQYVSSYLA | DASNRAT | QQRNNWPPT |
| PR305629 LC | RASQGITNYLA | AASTLQS | QKYNSAPWT |
| PR305633 LC | RASQSINSNLA | GASTRAT | QQYNNWPLT |
| PR305667 LC | RASQSISSYLN | AASSLQS | QQSYSTPLT |
| PR305684 LC | RASQGISSYLA | VASTLQS | QQLNNYPT |
| PR305689 LC | RASQSDSSSYLA | GTSSRAT | CQYGSSPLT |
| PR305734 LC | QASQDISNYLN | DASNLET | QQYDNLPLT |
| PR305754 LC | RASQSVSSYLA | DTSNRAT | QQRSNWPIT |
| PR305764 LC | RASQSVSSNLA | GASTRAT | QQYSDWPT |
| PR305754-P LC | RASQSVSSYLA | DTSNRAT | QQRSNWPIT |

TABLE 4A
Nucleic acid sequences of heavy chain (HCDR) complementarity
determining regions.

| Antibody ID | CDR1 | CDR2 | CDR3 |
|---|---|---|---|
| PR305618 | AGCTATGCC | GCTATTAGTGATAGTGGTGG | GAGGGTTTGATTACGTTTG |
| | ATGAGC | TAGCACATACTACGCAGACT | GGGGAGTTATCGTTATAGG |
| | | CCGTGAAGGGC | CTACTTTGACTAC |
| PR305623 | AGCTATTGG | AATATAAAGCAAGATGGAAG | CGTGGGAACTTCTTCTTTG |
| | ATGAGC | TCAGAAATACTATGTGGACT | ACAAT |
| | | CTGTGAAGGGC | |
| PR305629 | AATCACTAC | TATATCTATTACAGIGGGAG | GGGTTTGCTTAC |
| | TGGAGC | CACCAACTACAACCCCTCCC | |
| | | TCAAGAGT | |
| PR305633 | AGCTATGCC | GCTATCAGTGGCAGTGGTGA | GAGTATTACGATATTTTGA |
| | ATGAAC | TAGCACATACTCCACAGACT | CTGGTTATTGGGACTGGTA |
| | | CCGTGAAGGGC | CTTCGATCTC |
| PR305667 | AGCTATGGC | GTTATGTGGTATGATGGAAG | GGGTACGATATTTTGACTG |
| | ATGCAT | TGATAGATACTCTGCAGACT | GTCCCGACCACTTTGACTA |
| | | CCGTGAAGGGC | C |
| PR305684 | AGCTATGAC | ACTATTAGTGGTAGTGGTAG | GAGGGGGGAGCTACTGCTT |
| | ATGAAC | TCACACATACTACGCAGACT | TTGATATC |
| | | CCGTGAGGGGC | |
| PR305689 | ACCTATGGC | CTTATATGGTATGATGGAAT | CCCTATTACGATATTTTGA |
| | ATGCAC | TAATAAATACTATGCGGACT | CTGGTTATTTTGACTAC |
| | | CCGTGAAGGGC | |
| PR305734 | AGCTATGGC | GTTATATGGTATGATGGAAC | GATCCCTCCTTATGGTTCG |
| | ATGCAC | TAATAAATACTATGCAGACT | GGGAGTTCCCTCATTACTA |
| | | CCGTGAAGGGC | CGGTATGGACGTC |
| PR305754 | GGTTACTAC | GAAATCCAACATAGTGGAAG | CTAACTGGGGATTCCCTTT |
| | TGGAGC | CACCAACTACAAACCGTCCC | TGTTTGAGTAC |
| | | TCAAGAGT | |
| PR305764 | AGCTATGGC | GTTATATGGTATGATGGAAG | GATAGCGCCTCCGACTACT |
| | ATGCAC | TAATAAATACTATGCAGACT | TTGACTAC |
| | | CCGTGAAGGGC | |
| PR305754- | GGTTACTAC | GAAATCAATCATAGTGGAAG | CTAACTGGGGATTCCCTTT |
| P | TGGAGC | CACCAACTACAAACCGTCCC | TGTTTGAGTAC |
| | | TCAAGAGT | |

TABLE 4B
Nucleic acid sequences of light chain (LCDR) complementarity
determining regions.

| Antibody ID | CDR1 | CDR2 | CDR3 |
|---|---|---|---|
| PR305618 | CAGGCGAGTCAGG | GATGCATCCAATTT | CAACAGTATGATAAT |
| | ACATTAGCAACTA | GGAAACA | CTCCCGATCACC |
| | TTTAAAT | | |
| PR305623 | AGGGCCAGTCAGT | GATGCATCCAACAG | CAGCAGCGTAACAAC |
| | ATGTTAGCAGCTA | GGCCACT | TGGCCTCCGACG |
| | CTTAGCC | | |
| PR305629 | CGGGCGAGTCAGG | GCTGCATCCACTTT | CAAAAGTATAACAGT |
| | GCATTACCAATTA | GCAATCA | GCCCCGTGGACG |
| | TTTAGCC | | |
| PR305633 | AGGGCCAGTCAGA | GGTGCATCCACCAG | CAGCAGTATAATAAC |
| | GTATTAACAGCAA | GGCCACT | TGGCCGCTCACT |
| | CTTAGCC | | |
| PR305667 | CGGGCAAGTCAGA | GCTGCATCCAGTTT | CAACAGAGTTACAGT |
| | GCATTAGCAGTTA | GCAAAGT | ACCCCGCTCACT |
| | TTTAAAT | | |
| PR305684 | CGGGCCAGTCAGG | GTTGCATCCACTTT | CAACAGCTTAATAAT |
| | GCATTAGCAGTTA | GCAAAGT | TACCCCACT |
| | TTTAGCC | | |
| PR305689 | AGGGCCAGTCAGA | GGTACATCCAGTAG | CAGCAGTATGGTAGC |
| | GTGATAGCAGCAG | GGCCACT | TCACCGCTCACT |
| | CTACTTAGCC | | |
| PR305734 | CAGGCGAGTCAGG | GATGCATCCAATTT | CAACAGTATGATAAT |
| | ACATTACCAATTA | GGAAACA | CTCCCGCTCACT |
| | TTTAAAT | | |
| PR305754 | AGGGCCAGTCAGA | GATACATCCAACAG | CAGCAGCGTAGCAAC |
| | GTGTTAGCAGCTA | GGCCACT | TGGCCGATCACC |
| | CTTAGCC | | |
| PR305764 | AGGGCCAGTCAGA | GGTGCATCCACCAG | CAGCAGTATAGTGAC |
| | GTGTTAGCAGCAA | GGCCACT | TGGCCCACT |
| | CTTAGCC | | |
| PR305754-P | AGGGCCAGTCAGA | GATACATCCAACAG | CAGCAGCGTAGCAAC |
| | GTGTTAGCAGCTA | GGCCACT | TGGCCGATCACC |
| | CTTAGCC | | |

Further disclosed herein are antibody fragments, such as well-characterized Fabs (e.g., Fab, Fab′, F(ab′)₂, F(ab)₂,), Fvs (the variable region of the light chain and the variable region of the heavy chain expressed as two chains), and single-chain fragments (e.g., single-chain variable region fragments, scFv, scFv-Fc, and single chain Fabs, scFab), which also bind to EDIL3. Antibody fragments can include aptamers, minibodies, and diabodies. Methods of making these fragments are routine (see, e.g., Antibody Engineering: Methods and Protocols (Damien Nevoltris and Patrick Chames eds., 3d ed. 2018)).

The antibodies and fragments thereof disclosed herein can also include, but are not limited to, polyclonal, monoclonal, multispecific, human, humanized, or chimeric antibodies.

The phrase “complementary determining means” as used herein describes the six complimentary determining regions (CDRs) that collectively form specific interactions with EDIL3. CDRs within the scope of complementary determining means are the disclosed CDRs and functional equivalents thereto. Functional equivalent CDRs comprise different specific amino acid residues but maintain binding to EDIL3. Functional equivalent CDRs would differ insubstantially in their ability to disrupt EDIL3 from binding to LFA-1 integrin, for example, and therefore have a therapeutic effect.

Contemplated herein are conservative variants of the disclosed antibodies and fragments thereof. A protein is a conservative variant where it contains conservative amino acid substitutions that do not substantially affect or decrease the affinity of a protein. For example, an antibody that binds EDIL3 can include at least 1, 2, 5, 10, or 15 conservative substitutions, for example, in a constant domain, and bind the Edi13. Conservative amino acid substitution tables providing functionally similar amino acids are well-known to one of ordinary skill in the art. The following groups are examples of amino acids that are considered conservative substitutions for one another: 1) serine(S), threonine (T); 2) aspartic acid (D), glutamic acid (E); 3) asparagine (N), glutamine (Q); 4) arginine (R), lysine (K); 5) isoleucine (I), leucine (L), methionine (M), valine (V); and 6) phenylalanine (F), tyrosine (Y), tryptophan (W).

Herein, a “degenerate variant” can refer to a polynucleotide encoding a polypeptide (such as an antibody or fragment thereof) that includes a sequence that is degenerate based on the genetic code (i.e., the 20 natural amino acids can be specified by more than one codon). All degenerate nucleotide sequences encoding the disclosed antibody and fragment polypeptide sequences are included.

Further contemplated are variants of the disclosed antibodies and fragments thereof with a sequence identity of at least 60%, at least 70%, at least 80%, at least 85%, at least 90%, at least 95%, at least 98%, or at least 99% of the sequences according to Table 1A to Table 4B. Herein, “sequence identity”, “sequence homology”, or “sequence similarity” can refer to the similarity between amino acid or nucleic acid sequences, which is expressed as the similarity between the sequences. Sequence identity can be frequently measured as percent identity, in which two sequences are considered more similar the higher the percentage. Homologs or variants of a polypeptide or nucleic acid molecule possess a relatively high degree of sequence identity when aligned using standard methods, which are well-known. Ceslovas Venclovas, Methods for Sequence-Structure Alignment in Homology Modeling: Methods and Protocols, 55-82 (Andrew Orry and Ruben Abagyan, eds., 2012)).

Herein, “binding” (or “binds”) can refer to the well understood interaction between and antibody and a target protein, peptide, or polysaccharide. Binding can be measured in a variety of ways (see, e.g., Antibody Engineering: Methods and Protocols (Damien Nevoltris and Patrick Chames eds., 3d ed. 2018)). In embodiments, binding (e.g., binding activity) is measured by an ELISA assay. See Example 2, for example. A particular antibody or protein binds to a particular target protein, peptide, or polysaccharide and does not bind in a significant amount to other proteins or polysaccharides present in a sample or subject disclosed herein. Binding occurs between the disclosed antibodies and fragments thereof and an epitope of EDIL3. Herein, “epitope” can refer to discrete sites of an antigen recognized by the disclosed antibodies and fragments thereof. Epitopes may be linear or three-dimensional. The strength, or affinity of immunological binding interactions can be expressed in terms of the equilibrium binding constant (K_{d}) of the interaction, wherein a smaller K_{d} represents a greater affinity. An antibody binds to a target protein when the interaction has a K_{D} of less than 10⁻⁶ molar, such as less than 10⁻⁷ molar, less than 10⁻⁸ molar, less than 10⁻⁹ molar, or less than 10⁻¹⁰ molar.

The antibodies herein are monoclonal antibodies (“mAbs”). mAbs can be produced, for example, by hybridoma technologies, recombinant technologies, phage display technologies, synthetic technologies (e.g., CDR or specificity-determining residue, SDR, grafting), or combinations of such or other technologies known in the art. mAbs are antibodies derived from a single copy or clone including, for example, any eukaryotic, prokaryotic or phage clone. In some embodiments, EDIL3 antibodies can be generated using Single B cell cloning technology. A variety of well-known methods and tools can be used for producing and purifying the mAbs disclosed herein, including vectors, for example, plasmids, virus, or other vehicles for polynucleotide insertion or expression, and hosts, for example, microbial, yeast, insect, and mammalian organisms (see, e.g., Process Scale Purification of Antibodies (Uwe Gottschalk, ed., 2d ed. 2017)). For example, a vector for producing and purifying mAbs disclosed herein can contain a DNA segment encoding the monoclonal antibody described herein. In embodiments, the vector can be an adeno-associated virus (AAV), a retrovirus, a lentivirus vector, or the like.

Further antibodies having a complementary binding means can be prepared and screened by well-known methods, such as hybridoma, transgenic animals, and phage or yeast display (see, e.g., Monoclonal Antibodies: Methods and Protocols (Vincent Ossipow and Nicolas Fischer, eds., 2d ed. 2014)). Antibodies having equivalent complementary binding means differ in their amino acid sequence but perform the same function of binding the target through CDR-target interaction acting as (inhibitor/agonist/antagonist) to achieve the same result (inhibiting tumor growth). Preferably, the complementary binding means functions through the same epitope as the disclosed antibodies.

In embodiments, an antibody can comprise an Fc variant comprising an amino acid substitution which alters the antigen-independent effector functions of the antibody, in particular the circulating half-life of the antibody.

In embodiments, antibodies disclosed herein can comprise an Fc variant that can have reduced or eliminated glycosylation (e.g., N- or O-linked glycosylation).

In embodiments, antibodies disclosed herein can comprise an Fc variant that can either increase or decrease binding to FcRn when compared to antibodies lacking these substitutions, therefore, have an increased or decreased half-life in serum, respectively.

In embodiments, antibodies disclosed herein can comprise an Fc variant comprising mutations introduced to the constant regions of the mAb such that the antibody dependent cell-mediated cytotoxicity (ADCC) activity of the mAb is altered. For example, the mutation is a LALA mutation in the CH2 domain which reduces ADCC activity.

The antibodies and fragments thereof disclosed herein can be used in therapy. In embodiments, the antibodies and fragments thereof disclosed herein can be used to treat, prevent (such as through prophylactic treatment), or ameliorate a cancer or metastasis. In embodiments, the cancer can comprise melanoma, non-small-cell lung cancer (NSCLC), small cell lung cancer (SCLC), renal cell carcinoma (RCC), chronic lymphocytic leukemia (CLL; such as B cell CLL or T cell CLL), classical Hodgkin lymphoma (cHL), head and neck squamous cell carcinoma (HNSCC), colorectal cancer (CRC), gastric cancer, hepatocellular carcinoma (HCC), primary mediastinal large B-cell lymphoma (PMLBCL), bladder cancer, urothelial cancer, endometrial cancer, cervical cancer, breast cancer (e.g., triple negative breast cancer), Merkel cell carcinoma (MCC), and microsatellite instability high (MSI-H) or DNA mismatch repair deficient (dMMR) adult and pediatric solid tumors.

In some embodiments, the method for treating cancer can include the use of a pharmaceutically effective amount of one or more EDIL3 inhibitors that are administered to a subject with cancer. In some embodiments, the method of treating cancer leads to the reversal of immune excluded or immune dysfunctional tumor microenvironment. In some embodiments, the method for treatment of cancer decreases the immune suppressive effects of cancer-associated fibroblasts (CAFs) in a subject. In some embodiments, the method of treating cancer can counteract the effects of EDIL3-dependent blocking of LFA-1 binding to ICAM-1. In some embodiments, the method of treating cancer can counteract the effects of EDIL3-dependent blocking of LFA-1 binding to endothelial cells. In some embodiments, the EDIL3 inhibitor is an antibody or antigen-binding fragment or variant thereof that specifically binds EDIL3 protein.

In some embodiments, the method for treating cancer can include one or more additional therapies to treat cancer. In some embodiments, the additional therapy to treat cancer is a therapy that can modify angiogenesis. In some embodiments, the additional therapy to treat cancer is a therapy that can modify checkpoint blockade.

Herein, “preventing” a disease can refer to inhibiting the full development of a disease, such as cancer. “Treating” can refer to a therapeutic intervention that ameliorates a sign or symptom of a disease or pathological condition after it has begun to develop, such as a reduction in tumor burden or a decrease in the number of size of metastases.

“Ameliorating” can refer to the reduction in the number or severity of signs or symptoms of a disease, such as cancer. A “prophylactic” treatment is a treatment administered to a subject who does not exhibit signs of a disease or exhibits only early signs for the purpose of decreasing the risk of developing a pathology, such as cancer.

The antibodies and fragments thereof disclosed herein can be administered to subjects or patients. Herein, “administration” can refer to the act of the attending physician or caregiver, prescribing the agent for administration and thereby causing the application of an agent to a subject, through ingestion, infusion, injection, or any other means, whether self-administered or administered by a clinician or other qualified care giver. For example, administration of the mAb disclosed herein can abrogate or inhibit or interfere with an activity of the EDIL3 protein.

The term “therapeutic” in conjunction with antibody disclosed herein refers to an antibody suitable for use in human treatment of cancer. In embodiments, such an antibody can have a K_{D} of less than 10⁻⁶ molar, such as less than 10⁻⁷ molar, less than 10⁻⁸ molar, less than 10⁻⁹ molar, or less than 10⁻¹⁰ molar and any toxic or detrimental effects of the antibody are outweighed by the therapeutic beneficial effects.

Herein, a “subject” includes both human patient and veterinary subjects, including human and non-human mammals. In embodiments, the subject or patient has or has a risk of cancer.

A pharmaceutical composition of the present disclosure contains an “effective” or “therapeutically effective” amount, as used interchangeably herein, of a monoclonal antibody of the present disclosure. The dosages and dosage regimen to achieve the desired therapeutic result depending on the means of administration and may vary according to factors such as the disease state, age, sex, and weight of the individual, and the ability of the monoclonal antibody to elicit a desired response in the individual. The dosage administered to a subject (e.g., a patient) of the antigen-binding polypeptides described herein is typically 0.1 mg/kg to 100 mg/kg of the patient's body weight, between 0.1 mg/kg and 20 mg/kg of the patient's body weight, or 1 mg/kg to 10 mg/kg of the patient's body weight. An effective amount is also one in which any toxic or detrimental effects of the monoclonal antibody of the present disclosure are outweighed by the therapeutically beneficial effects.

A pharmaceutical composition of the present disclosure can be formulated to be compatible with its intended route of administration. Non-limiting examples of routes of administration include parenteral, e.g., intravenous, intradermal, subcutaneous, oral (e.g., inhalation), transdermal (i.e., topical), transmucosal, and rectal administration.

The pharmaceutically acceptable carriers of use are conventional (e.g., as described in Remington, The Science and Practice of Pharmacy, 22nd Edition, Loyd V., ed., Pharmaceutical Press, 2012). In general, the nature of the carrier will depend on the mode of administration. For instance, parenteral formulations typically comprise injectable fluids that include pharmaceutically and physiologically acceptable fluids, such as water, physiological saline, balanced salt solutions or the like as a vehicle. Pharmaceutical compositions can additionally include minor amounts of non-toxic auxiliary substances for stability.

In embodiments, the carrier can be sterile and/or suspended or otherwise contained in a unit dosage form including one or more measured doses of the composition suitable for administration to a subject of an effective amount of the antibodies and fragments thereof disclosed herein. Medications for use in treatment can also be included in embodiments. In embodiments, the unit dosage form can be in a sealed vial that contains sterile contents or a syringe for injection into a subject, lyophilized for subsequent solubilization and administration, or in a solid or controlled release dosage.

Herein, an “effective amount” can refer to a quantity sufficient to achieve a desired effect in a subject. For instance, this can be the amount necessary to prevent, treat, or ameliorate a disease, for example, inhibiting or suppressing cancer. In embodiments, an effective amount is the amount necessary to eliminate, reduce the size, or prevent metastasis of cancer or a tumor. Efficacy is first evident in the cellular response, for which a variety of in vitro and cell assays are well-known to measure. Kristina V. Kitaeva et al., Cell Culture Based In vitro Test Systems for Anticancer Drug Screening, 8 Front. Bioeng. Biotechnol. 322 (2020)). In embodiments, an effective amount is the amount necessary to significantly inhibit or reduce cancer cell proliferation or migration, invasion, or adhesion. A cellular response manifests as significantly reduced tumor size, reduced or inhibited disease progression, and improvement in survival in a subject. More particularly, an effective amount provides improvement in important cancer endpoints, Overall Survival (OS), Disease-Free Survival (DFS), Objective Response Rate, Complete Response Rate or Progression Free Survival (PFS). See Dept. of Health and Human Services, Food and Drug Admin, Clinical Trial Endpoints for the Approval of Cancer Drugs and Biologics: Guidance for Industry (2018); E. A. Eisenhauer et al., New Response Evaluation Criteria in Solid Tumours: Revised RECIST Guideline (Version 1.1), 45 Eur. J. Cancer 228 (2009).

The mAbs disclosed herein can be used in conventional methods relating to the localization and/or quantitation of an EDIL3 protein (e.g., for use in measuring levels of the EDIL3 protein within appropriate physiological samples, for use in diagnostic methods, for use in imaging the protein, and the like).

The mAbs disclosed herein can be used in methods relating to the isolation of an EDIL3 polypeptide by conventional techniques, such as immunoaffinity, chromatography or immunoprecipitation. Antibodies directed against an EDIL3 protein (or a fragment thereof) can be used diagnostically to monitor protein levels in tissue as part of a clinical testing procedure, e.g., to, for example, determine the efficacy of a given treatment regimen.

Cancer-associated fibroblasts (CAFs) are a group of activated fibroblasts with significant heterogeneity and plasticity in the tumor microenvironment that secrete a variety of factors to regulate tumor occurrence, development, metastasis, and therapeutic resistance. Accordingly, aspects are drawn towards a method of decreasing immune suppressive effects of cancer-associated fibroblasts (CAFs) in a subject. Decreasing, reversing, or modulating immune suppression can refer to altering, impeding, reducing the immunosuppressive properties of cancer-associated fibroblasts. In embodiments, the immune suppressive effects of CAFs can be reduced in a mammal by 1%, 2%, 3%, 4%, 5%, 10%, 20%, 30%, 40%, 50%, 60%, 70%, 80%, 90%, 100% as compared to a control sample.

For example, the antibody is an EDIL3 antibody. In embodiments, the method further comprises measuring EDIL3 serum levels. In embodiments, a decrease in immunosuppressive CAF activity can be measured via immune infiltration or cytokine changes in the tumor microenvironment.

Aspects are drawn towards a method of identifying a subject non-responsive to immune checkpoint blockade therapy. For example, embodiments, comprise detecting EDIL3 expression or protein levels in a sample obtained from a subject. “Detecting” can refer to quantitative, semi-quantitative, qualitative, or other methods for determining an analyte, e.g., EDIL3, in a sample. For example, detecting can comprise conventional methods, such as immunohistochemistry of the tumor and/or the tumor microenvironment; measuring levels of EDIL3, such as circulating EDIL3; and/or measuring levels of EDIL3 antibody. In embodiments, the elevated levels of EDIL3 expression or protein can be indicative of a subject non-responsive to immune checkpoint blockade therapy.

In embodiments, the EDIL3 expression or protein levels can be compared to a control sample and, if changed when compared to the control sample, the subject can be identified as non-responsive to immune checkpoint blockade therapy or responsive to immune checkpoint blockade therapy. As used herein, “changed as compared to a control” sample or subject is understood as having a level of the analyte or diagnostic or therapeutic indicator (e.g., marker) to be detected at a level that is statistically different than a sample from a normal, untreated, or abnormal state control sample. Determination of statistical significance is within the ability of those skilled in the art, e.g., the number of standard deviations from the mean that constitute a positive or negative result.

The mAbs of the present disclosure can also be used in combination therapy. In embodiments, the subject is treated with an mAb disclosed herein in combination with one or more additional therapies to treat cancer, for example, radiation, surgery, bone marrow transplantation, chemotherapy, immunotherapy, hormone therapy, or targeted therapy. Use of the mAbs of the present disclosure in combination with chemotherapy or immunotherapy is preferred. Most preferably, the additional treatment is directed to angiogenesis and/or checkpoint blockade. Accordingly, aspects of the invention are drawn towards administering an mAb disclosed herein in combination with an anti-angiogenic agent and/or an immune checkpoint blockade therapy.

As used herein, “combination” therapy or use in combination refers to the administration of the mAbs of the present disclosure to a patient in conjunction with (i.e., before, simultaneously, or following) any number of relevant treatments. Such treatments include, but are not limited to, agents such as an angiogenesis inhibitor and/or an immune checkpoint inhibitor.

“Angiogenesis” can refer to the formation and spread of blood vessels. For example, “angiogenesis” can refer to a process involving tissue angiogenesis, such as, the proliferation, migration and invasion of vascular endothelial cells and the development of new capillaries.

An “angiogenesis inhibitor” or “anti-angiogenic agent” can refer to a substance that inhibits the growth of new blood vessels. In embodiments, the angiogenesis inhibitor can comprise cytokines, an EDIL-3 antibody, a VEGF inhibitor, a tyrosine kinase inhibitor, or a combination thereof. For example, the VEGF inhibitor comprises a VEGF antibody. For example, the angiogenesis inhibitor comprises axitinib, bevacizumab, cabozantinib, everolimus, lenalidomide, Lenvatinib mesylate, pazopanib, ramucirumab, regorafenib, sofafenib, sunitinib, thalidomide, vandetanib, and ziv-aflibercept.

Without wishing to be bound by theory, administering an angiogenesis inhibitor can synergize a patient's response to an immune blockade therapy.

The term “immune checkpoint inhibitor” or “immune checkpoint blockade therapy” can refer to any compound inhibiting the function of an immune inhibitory checkpoint protein. Inhibition includes reduction of function and full blockade. In embodiments, immune checkpoint inhibitors can be antibodies that specifically recognize an immune checkpoint protein. In embodiments, immune checkpoint inhibitors can include peptides, antibodies, nucleic acid molecules, and small molecules.

In embodiments, the checkpoint blockade therapy can comprise a CTLA4 antibody, a PD-L1 antibody, a PD-1 antibody, a LAG-3 antibody, or a combination thereof. For example, the CTLA4 antibody can comprise ipilimumab, tremelimumab, or a combination thereof. For example, the PD-L1 antibody can comprise atezolizumab, avelumab, duvalumab, or a combination thereof. For example, the PD-1 antibody can comprise pembrolizumab, nivolumab, cemiplimab, or a combination thereof. For example, the LAG-3 antibody can comprise relatlimab.

### EQUIVALENTS

Various aspects of the present disclosure may be used alone, in combination, or in a variety of arrangements not specifically discussed in the embodiments described in the foregoing and is, therefore, not limited in its application to the details and arrangement of components set forth in the foregoing description or illustrated in the drawings. For example, aspects described in one embodiment may be combined in any manner with aspects described in other embodiments.

### EXAMPLES

Examples are provided below to facilitate a more complete understanding. The following examples illustrate the exemplary modes of making and practicing the invention. However, the scope is not limited to specific embodiments disclosed in these Examples, which are for purposes of illustration only, since alternative methods can be utilized to obtain similar results.

### Example 1

### EDIL3 as an Angiogenic Target of Immune Exclusion

### Abstract

Immune checkpoint blockade (ICB) has become a standard care for a number of solid tumors. To improve these clinical results, multiple combinatorial approaches have been studied. The combination of anti-angiogenesis and immune checkpoint blockade has demonstrated efficacies in several cancers. To improve the mechanistic understanding of synergies with these treatment modalities, serologic screening of human protein arrays were performed utilizing sera from long-term responding patients treated with ipilimumab plus bevacizumab (Ipi-Bev). High-titer antibody response against EDIL3 were identified which correlated with favorable clinical outcomes. EDIL3 is a secreted ECM protein involved in carcinogenesis and identified as a marker of poor prognosis in various malignancies. EDIL3 can be associated with immune exclusion signatures for cytotoxic immune cell infiltration and non-response to ICB by Tumor Immune Dysfunction and Exclusion (TIDE) analysis. Among cells involved in immune exclusion, cancer associated fibroblast (CAFs) can be a primary source of EDIL3. Further TCGA and Checkmate 064 expression analyses correlated high levels of EDIL3 with increased TGF-β signaling in fibroblasts, enrichment of angiogenic signatures, and induction of EMT. EDIL3 overexpression and TGF-β1 regulation were validated in patient-derived CAFs. Silencing EDIL3 in CAFs disrupted TGF-β1 induced EMT. Circulating levels of EDIL3 correlated with VEGF in patient serum samples. Like VEGF, EDIL3 promoted migration and tube formation ability of patient derived-tumor endothelial cells (TECs). Mechanistically, 3-D Microfluidic Cultures and 2D transmigration assays with TECs endorsed EDIL3 mediated disruption of LFA-1 and ICAM1 interaction as a means of T-cell exclusion. EDIL3 can be a target for improving immune cell endothelial transmigration and efficacy of ICB therapy.

### Introduction

Immune checkpoint blockade has become standard of care for numerous malignancies including melanoma for which approximately 50% of patients with metastatic disease receive benefit. Combinatorial approaches to improve outcomes and overcome treatment resistance are sought for a significant patient population with unmet need. The immune system is known to have an active interplay with the vascular system, as it is the gateway to the tumor microenvironment. Angiogenic factors have indicated immune suppression capabilities and tumor blood vessels can limit access of immune effector cells to the tumor microenvironment. Innovative strategies including the combination of immune checkpoint blockade (ICB: Immune Checkpoint Blockade) with anti-angiogenic treatment have revealed significant efficacy in a number of tumor types including renal cell carcinoma, hepatocellular carcinoma, endometrial cancer, non-small cell lung cancer, and melanoma. Correlatives revealed that VEGF blockade is associated with improved lymphocyte trafficking across the endothelium as well as improved outcomes when tumor assessment had a myeloid expression signature.

Induction of humoral immunity to vascular antigen targets Gal-1 (Galectin-1) (1), Gal-3 (Galectin-3) (1, 2) and ANGPT2 (Angiopoietin2) (3) were observed with ipilimumab and bevacizumab treatment that were associated with favorable outcomes. To better understand the immunologic role of combination therapy, sera from patients with long-term benefits were utilized to screen human protein arrays.

### Materials and Methods

### Blood Sampling and Sera of Melanoma Patients

The phase I Ipi-Bev trial conducted on advanced melanoma patients has been reported previously (1, 2). Briefly, Heparinized peripheral blood samples were obtained from melanoma patients participating in IRB-approved protocols at the Dana-Farber/Harvard Cancer Center. The blood samples were processed same day by standard gradient centrifugation using Ficoll-Paque Plus (GE Healthcare Biosciences). The upper phase was then transferred into 1.5 mL tubes and then centrifuged at 12000 g for 10 min at 4° C. The resulting plasma was aliquoted and stored at −80° C. until use. Studies of humoral responses were primarily based on four cohorts of advanced melanoma patients who were treated with i) ipilimumab alone, ii) ipilimumab plus bevacizumab, iii) anti-PD-1 alone or iv) nivolumab plus ipilimumab (Nivo+Ipi) as standard of care or through participation in the Ipi-Bev clinical trial.

### ELISA Experiments

For detection of antibodies in plasma, ELISA was carried out as previously described (1). Briefly, 96-well high binding microtiter plates (Corning) were coated overnight with 100 ng of the purified human recombinant proteins (rhEDIL3 and rhMFGE8 in 50 μl of TBS (Tris Buffered Saline)) and, using His tag and no plasma as negative controls. After washing three times with Phosphate Buffered Saline (PBS) with 0.05% Tween 20 (PBST), plates were blocked with blocking buffer (Pierce Protein-Free blocking buffer) for 2 h at room temperature. Plasma samples (dilution, 1:500-1:1000 in blocking buffer) were incubated for 1.5 h at 4° C. After washing, HRP-labeled anti-human IgG (Jackson laboratories) (dilution, 1:4000 in 1% BSA (Bovine Serum Albumin)-PBST) was added for 1 h at room temperature. To enhance the sensitivity of the immunoassay, the ELAST amplification system was used (Perkin-Elmer). The signal was developed with TMB (3, 3′, 5, 5′-Tetramethylbenzidine, Sigma) substrate for 5 min. The reaction was stopped with 1N HCl, and absorption measured at 450 nm and 570 nm. Antibody titer was calculated by subtracting OD (optical density) 570 nm from OD 450 nm and subtracting “His Tag” background and “No Plasma” background from EDIL3/MFGE8 reading. The bars represent average absorbance obtained from the duplicate samples.

### Measurement of Circulating EDIL3

Secreted EDIL3 levels in patient's plasma were determined in duplicate using human EDIL3 ELISA kit (R&D Systems) according to the manufacturer's instructions. A standard curve was constructed for each assay.

### Immunoblot Blot

For detection of EDIL3 antibody in plasma, equal amounts of human recombinant EDIL3 protein denatured in 2×SDS-containing-Laemmli buffer were resolved by SDS-PAGE and transferred to PVDF membrane. Each lane of the membrane was cut and immunoblotted with pre-treatment and post-treatment plasma samples in a separate container. Plasma samples were diluted in 1% BSA in TBS with 0.1% Tween-20 (TBST) at 1:500 dilutions. Proteins were visualized using ECL kit (Perkin Elmer) and digitally processed using ImageQuant LAS-4000. Membranes were stripped and re-probed with commercial EDIL3 antibody (Abcam) for loading controls.

### Bioinformatics Data Analysis

EDIL3 and MFGE8 were the identified targets from protein array screens with plasma samples from melanoma patients (1). The immunologically important glycoproteins EDIL3 and MFGE8 were subjected to a computational signature analysis for T cell dysfunction and T cell exclusion using TIDE (Tumor Immune Dysfunction and Exclusion) analysis (3). CTNNB1 (β-catenin) was included as a reference marker for immune exclusion (4).

The Cancer Genome Atlas (TCGA) and BMS CheckMate 064 datasets were accessed and analyzed gene expression grouped by TGFβ, EMT and angiogenesis signatures (PMID: 29443960). The rows of the heatmap shows expression (z scores) of genes of interest. Melanoma cases from TCGA [PMID: 26091043] (n=469) and BMS-064 [PMID: 30021886] pre-treatment patients (n=90) with RNA-sequencing are shown ordered by EDIL3 expression from low (left) to high (right). Gene expression data was log 2 transformed. Pathway expression was calculated using the GSVA [PMID: 23323831] tool for TGF-β (TGF-beta), pan-fibroblast TGF-β response signature (F-TBRS), EMT and angiogenesis (Angio) gene sets described in Mariathasan et. al. (5).

### Cell Lines

Primary TECs were isolated from melanoma patient enrolled in the Ipi-Bev trial following Institutional Review Board approved protocols. Human umbilical vascular endothelial cells (HUVECs) were purchased from ATCC. Endothelial cells were maintained in endothelial basal medium (EBM-2) (PromoCell, Heidelberg, Germany) and were used within 3-4 passages of thawing. CAFs were isolated from tumor sample (6). Normal fibroblasts (NFs) were purchased (ATCC). CAFs and NFs were maintained in DMEM medium with 10% FBS and 1% antibiotics (Gibco-BRL).

Peripheral blood mononuclear cells (PBMCs) were isolated from normal donor blood samples using Ficoll density gradient separation per IRB approved protocols. T cells were isolated from PBMC using Pan T Cell Isolation Kit (Miltenyi Biotech). T cell activation was achieved by adding human monoclonal anti-CD3 (Biolegend) and anti-CD28 (Biolegend) antibodies in RPMI media supplemented with 10% FBS, 50 μg/ml penicillin, 100 μg/ml streptomycin) and 30 U/ml IL-2 as guided by the manufacturer (Life Technologies).

RNA Extraction and Real Time Quantitative PCR (qPCR)

RNA was extracted using RNeasy mini kit (Qiagen) and reverse transcribed into cDNA using SuperScript VILO cDNA Synthesis Kit per manufacturer's instructions (Invitrogen). Gene expression levels were analyzed by qPCR using primers EDIL3 (h)-PR (sc-91971-PR). A standard program at ABI 7500:95° C. for 10 min, 40 cycles at 95° C. for 15s and 60° C. for 1 min was used. β-actin was used as reference.

### EDIL3 Silencing

CAFs or TECs were seeded in 6-well plate at 80% confluency a day before transfection. The cells were transfected either with control siRNA-A (sc-37007) or siEDIL3 (sc-91971) using Lipofectamine™ RNAiMAX Transfection Reagent (Thermofisher scientific) in MEM (Corning) per manufacturer's instructions. After 6 h the media was changed to default growth media of the cells and cultured overnight. The silencing of EDIL3 was confirmed by qRT-PCR or immunoblot analysis as described.

### Wound Healing Assay

Wound healing assay was performed as reported previously (9). Briefly, TECs were seeded in fibronectin coated 12-well plates and allowed to grow till confluent. On 90% confluency cells were starved in low serum conditions for 24 h followed by wounding across the center of the well with a sterile 200 μl pipette tip followed by incubation in control, VEGF, or EDIL3 supplemented media. Using a phase contrast microscope, the leading wound edges were photographed at 0 h, 24 h and 48 h at the same reference areas for each well. Three independent experiments were conducted, each treatment in triplicate with three areas of reference per replicate. The percent wound area was calculated by using ImageJ software.

### Tube Formation Assay

The endothelial tube formation assay was performed using Cultrex In Vitro Angiogenesis Assay (R&D systems) as per protocol. Briefly, the TECs were seeded in Cultrex® RGF BME coated wells of 96 well plate at cell density 30×10³ per well in triplicate in EBM-2 media. The Cultrex®RGF BME vial was thawed on ice one night before the seeding. In a chilled 96 well plate Cultrex® RGF BME was added at 50 μl per well using a chilled 200 μl pipette tip. The plate was centrifuged at 250 g for 5 min at 4° C. to allow the BME to spread evenly. To solidify the BME the coated plates were kept for 30 min at 37° C. Meanwhile cells were collected and diluted in conditioned media as control, VEGF or EDIL3 supplemented. The cells were seeded and incubated at 37° C. for 12 h followed by imaging using Nikon Eclipse TE2000-Smicroscope at 4× magnification.

### Surface Flow Cytometry Analysis for LFA-1

THP-1 cells grown in suspension in RPMI-1640 were pre-activated with PMA 1 ng/mL for 24 h and rested overnight. Human Jurkat T cells were grown in log phase in RPMI-1640 medium. Primary human Pan-T cells were isolated from PBMCs derived from healthy donors by negative selection using Pan T Cell Isolation Kit (Miltenyi Biotech). Isolated T cells were stimulated with human T-Activator CD3/CD28 in RPMI medium supplemented with IL-2 (100 IU/mL). The cells were counted and maintained 1-2×10⁶ cells/mL by adding fresh RPMI medium with IL-2 every 2-3 days. On day 9, the cells were re-stimulated with human T-Activator CD3/CD28. The expanded T cells were used for flow cytometer analysis for verification of LFA-1 expression. Briefly, PMA activated THP-1, Jurkat T cells, and expanded human T cells were stained with FITC-conjugated CD11a (BioLegend) or isotype control. The cells were washed twice with FACS buffer and resuspended in fixation buffer with 1.6% PFA in PBS. The cells were acquired on a BD LSRII flow cytometer using FACSDiva software (BD Biosciences). The data were analyzed by FlowJo software.

### Adhesion Assay

Adhesion assay was performed as described with modifications (7, 8). At a seeding density of 15×10³ cells per well, endothelial cells were grown on fibronectin coated 96-well plates. After 24 h of seeding the cells were treated with BSA or stimulated by adding TNFα (5 ng/ml) for another 24 h. T-cells/THP-1 cells were labelled with BCECF (1 μM, Life Technologies) for 30 min followed by incubation with respective concentrations of rEDIL3 for 1 h at 37° C. in dark or low light conditions. The endothelial monolayers were washed three times with PBS at the end of the treatment. Pre-treated labelled T-cell/THP-1 cells were added at 50×10³ cells/100 μl to all the wells and allowed to adhere for 45 min at 37° C. Fluorescence was measured at 490 nm before (input) and after four washes of warm RPMI media to remove nonadherent cells usingSpectraMax®M3 Multi-Mode Microplate Reader. The percentage of adhesion is defined as the fluorescence of adherent cells divided by the fluorescence of cell input.

### EC Transmigration Assay

For EDIL3 knockdown, TECs were transfected as described for 24 h prior to seeding in 24-well transwells. Endothelial cells were grown for 48 h to form monolayers onto fibronectin coated polycarbonate membrane of 5.0 μM pore size and 6.5 mm insert diameter in 24-well transwell plates (Costar). Pre-activated T cells labelled with BCECF (1 μM, Life Technologies) were pre-treated with BSA or rEDIL3 for 1 h at 37° C. The endothelial monolayers were gently rinsed with warm RPMI-1640 media before adding the pretreated T cells at 1×10⁵ cells/100 μl in each insert. These inserts were transferred to a new 24 well plate with lower chambers containing 0.6 ml of RPMI-1640 media supplemented with or without the chemoattractant IP-10 and incubated for 2-4 h at 37° C. T cells trans-endothelial migration was assessed by measuring the fluorescence at 490 nm using SpectraMax® M3 Multi-Mode Microplate Reader with and without the inserts in the respective wells of the transwell plate.

### 3D Vascular Model

To generate the tumor-vascular model, H226 spheroids mixture with collagen rat tail hydrogel mixture (2.5 mg/ml) was injected into the center gel region of the 3D microfluidic chamber (10-15 μL per each microfluidic chamber) as source of IP-10. After incubation for 30 min at 37° C. in sterile humidity chambers. All the side walls of one flanked channel (media channel) were coated with a 150 μg/ml collagen solution in PBS to allow a better adhesion of TECs to the channel. After 15 min, channels were washed once with medium.

To create the 3D vessels for Tumor-vascular model or 3-D vascular model, 25 μL cell suspension of 3×10⁶ cells/ml of patient-derived TECs were injected in the media channel coated with collagen. The fluidic channel chip was rotated twice to create a confluent hollow-lumen 3D vessel. To allow the cells to attach to the media-gel interface to form a monolayer, the chip was placed with face down for 15 min with the media-gel interface would face down. A 50 μL cell suspension was reinjected, and the chip was faced upside down to cover the upper part of the 3D vascular channel. After 90 min of incubation in the humidity chamber at 37° C., cell culture medium was gently added to both media channels. Chips were placed in the incubator to form a confluent monolayer. After vascular formation in the tumor-vascular model or 3-D vascular model, CD3/CD8 positive T cells (labelled with cell tracker) pre-treated for 1 h with EDIL3 at 37° C. and finally loaded in EDIL3 supplemented media were added at 2:1 TEC:T ratio. For 3-D vascular model, cells IP-10 supplemented media was added in the fluidic channel placed on the opposite site of the vascular barrier. T cells migration across blood vessel in the middle chamber were quantified at 48-72 h using name/model/make of the microscope.

### Statistical Analysis

### Bioinformatics and Clinical Data

All data are presented as mean±SD, and was analyzed using an unpaired, 2-tailed Student's t test. P value of 0.05 or less was considered statistically significant. In vitro assays analysis was performed using the GraphPad Prism software version 9.3.1.

### Results

### Ipi-Bev Therapy Elicited Functional Humoral Response to EDIL3 and MFGE8

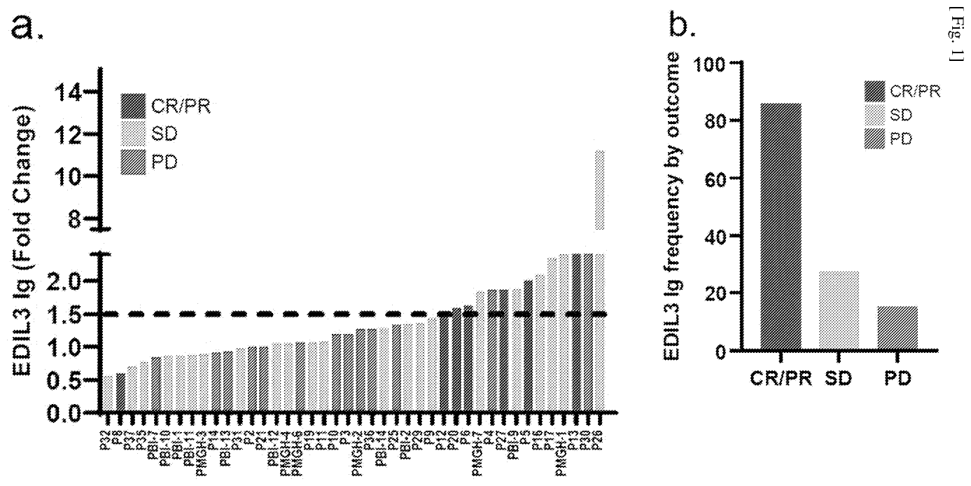

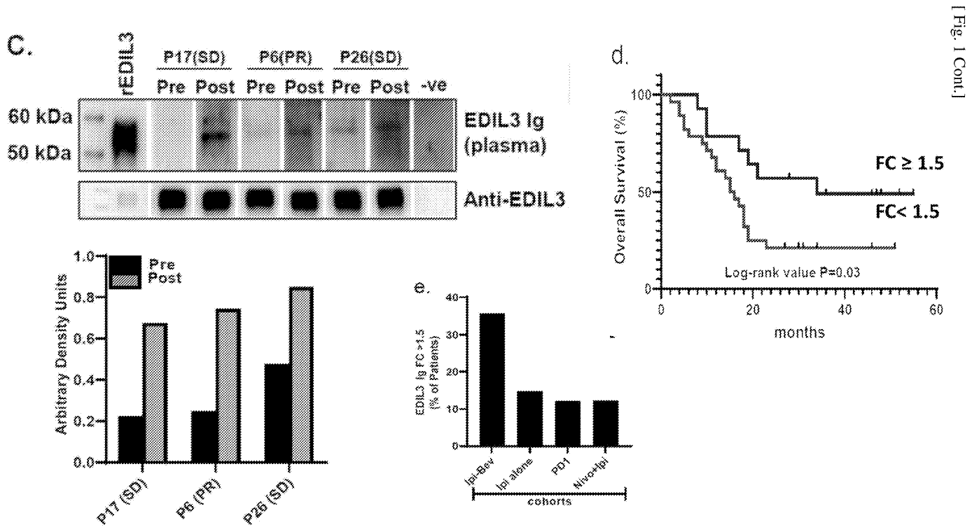

The pre-treatment and post-treatment plasma samples from 42 advanced melanoma patients treated with Ipi-Bev combination were screened by protein array (data not shown). As per manufacturer's instructions proteins with Z-factor of 0.4 or greater were weighed as targets. Antibody responses against EDIL3 and MFGE8 were detected in subsets of patients (Data not shown). To determine whether the humoral responses were associated with clinical outcomes, the patients were divided based on antibody titer into high and low groups with cut-off of 50% i.e., fold change (FC) 1.5 as significant (FIG. 1 panel a). Patients with improved clinical response were found to have significantly higher FC for EDIL3 antibody than non-responders (median FC, responders vs. non responders: 2.5 vs. 1.0, p=0.01). Six of the 7 responders showed significant increase (FC≥1.5) in anti-EDIL3 IgG titers. Overall patients with complete or partial response (CR/PR) by RECIST criteria to the Ipi-Bev combination therapy had a significant increase in anti-EDIL3 IgG response followed by patients with stable disease (SD) and progressive diseases (PD) in descending order of anti-EDIL3 IgG titers respectively (CR/PR=85.71% vs SD=31.82%; PD=15.38%) (FIG. 1 panel b). Thus, the humoral response for EDIL3 was significantly correlated with higher response rate to Ipi-Bev combination therapy. However, the humoral response towards MFGE8 did not correlate with clinical outcomes and was found to be associated with PD (Data not shown) and no change in overall survival was observed (Data not shown). The expression and increase in anti-EDIL3 IgG antibody was further confirmed by performing immunoblot assay (FIG. 1 panel c) and ELISA (Data not shown) with pre-treatment and post-treatment plasma of representative responders from Ipi-Bev patient cohort. The anti-EDIL3 antibody response was also associated with better overall survival (OS) of patients in the Ipi-Bev cohort (long rank p<0.027). The median survival of the patients with EDIL3 antibody FC<1.5 was 70 weeks (95% CI, 47-81), whereas that of patients with FC>1.5 was not reached (FIG. 1 panel d).

To further study the humoral response to EDIL3 as the function of treatment, the FC of anti-EDIL3 antibody titers in the pre-treatment and post-plasma samples of patient's cohorts treated with Ipilimumab alone (n=34), anti-PD1 alone (n=25) and Nivolumab-Ipilimumab combination (n=41) were analyzed (FIG. 1 panel e). Ipi-Bev cohort had the highest percentage (35.7%) of patients with significant FC (>1.5) of IgG titers for anti-EDIL3 antibody, while other cohorts had comparable FC in patients (14% for Ipi alone, 12% for anti-PD1 and 12.2% for Ipi+Nivo).

EDIL3 and MFGE8 Expression Correlates with Tumor Immune Exclusion

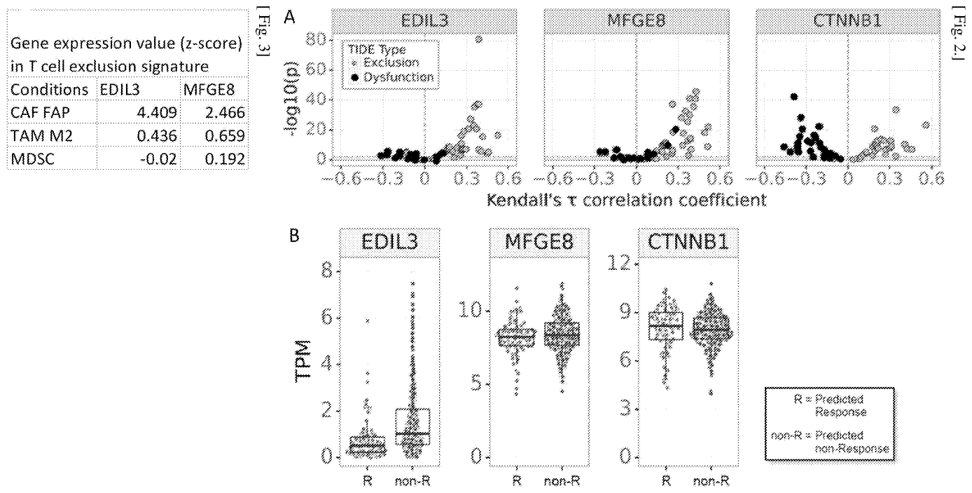

TIDE analyses, a transcriptome biomarker platform for assessing ICB response by inferring gene functions in regulating tumor immunity (5), predicted T cell exclusion phenotypes and not dysfunction mediated by increased EDIL3 and MFGE8 expression. Beta-catenin i.e., CTNNB1, was used as a reference gene for TIDE analysis. CTNNB1 is known to lead T-cell exclusion and resistance in melanoma thus, a biomarker for predicting the ICB response in patients (6, 7). The volcano-plots for EDIL3 and MFGE8 expression in melanoma patients depicted a high association with T cell exclusion, while the volcano-plot for β-catenin was congruous with exclusion as well as dysfunction phenotype (FIG. 2 panel a). In addition, high EDIL3 expression was also found to be significantly associated (p=7.4e-11) with predicted-worse ICB outcomes in melanoma patients by TIDE analysis while MFGE8 and CTNNB1 were not associated with ICB response (FIG. 2 panel b). Thus, EDIL3 expression correlates with T-cell exclusion and a predicted non-response to ICB therapy in melanoma patients.

EDIL3 Mediated T-Cell Exclusion is Associated with TGFβ Signaling, EMT, and Angiogenesis Signatures

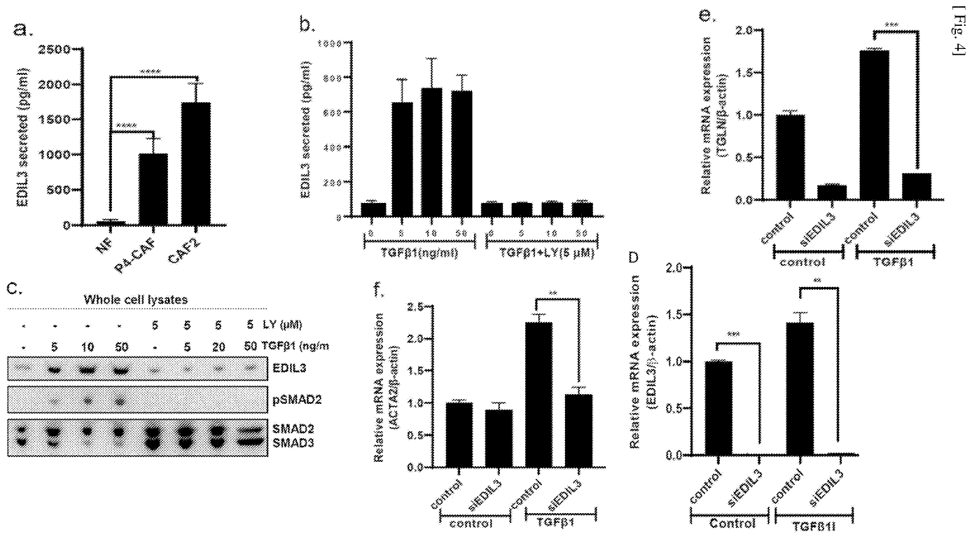

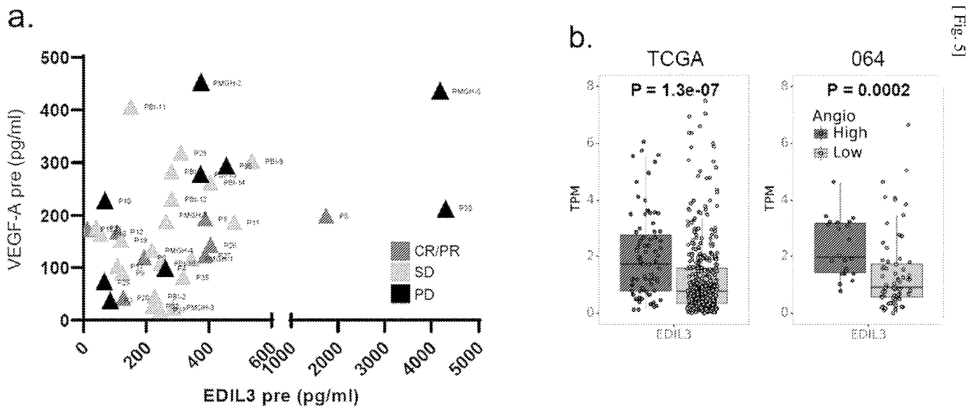

The gene query tool on TIDE platform was used to evaluate its expression in immunosuppressive cell types that drives T cell exclusion in TME. Among all the cell types promoting T-cell exclusion, CAFs were found to have very high EDIL3 expression levels followed by tumor associate macrophages (TAM) of M2-like phenotype. However, myeloid derived suppressor cells (MDSCs) were negatively associated with EDIL3 expression. Interestingly, expression of MFGE8 was also found to be strongly associated with CAFs and weakly with TAM of M2-like phenotype and MDSCs (FIG. 3).

TCGA Skin Cutaneous Melanoma (SKCM) dataset (PMID: 26091043) cohort utilized all 469 primary and/or metastatic melanoma samples with RNA-seq data available. In addition, the Checkmate 064 dataset analysis utilized all 90 pre-treatment samples from both study arms (PMID: 30021886) with RNA-seq data available. With an increasing EDIL3 expression in the TCGA SKCM subjects, a significant enrichment of TGFβ signaling, pan-fibroblast TGF-β response signature, EMT phenotype, and angiogenic signatures were observed (Data not shown). The TCGA SKCM analysis was next contrasted with the CheckMate 064 dataset generated from subjects with advanced or metastatic melanoma undergoing an open label, randomized, phase 2 study of nivolumab given sequentially with ipilimumab. Expression profile of EDIL3 along with the gene sets of selected pathways from TCGA SKCM analysis mentioned above were visualized as a heatmap for 90 samples from the CheckMate 064 dataset. The CheckMate 064 dataset analysis confirmed the previous observations from TCGA SKCM analysis i.e., when arranged in increasing order of EDIL3 expression pathway scores for TGFβ signaling, pan-fibroblast TGF-β response signature, EMT phenotype, and angiogenic signatures were positively correlated (Data not shown). Thus, our analysis indicated immunosuppressive CAFs as source of EDIL3 and its role in regulating TGF-β signaling, EMT phenotype, and angiogenesis in TME of advanced melanoma patients.

EDIL3 is Abundantly Expressed in CAFs Isolated from Melanoma Patient's Tumor Biopsies and Linked with TGF-β1 Induced EMT

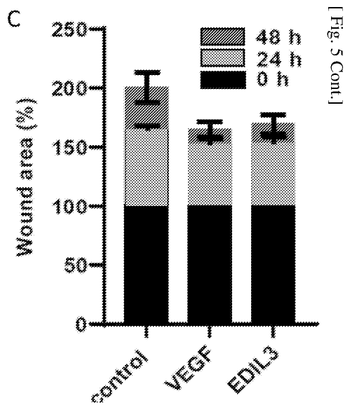

Since TIDE gene query analysis indicated the EDIL3 expression in CAFs, EDIL3 expression was analyzed in fibroblasts. As compared to NFs, the patient derived CAFs (P4-CAF and CAF2) secreted abundant levels of EDIL3 in the medium as detected by ELISA (P4-CAF (p<0.0001); CAF2 (p<0.0001)) (FIG. 4, panel a). Next, NFs were pretreated with or without LY2109761 followed by induction with TGFβ1 for 24 h. TGFβ1 treatment activated downstream SMAD signaling in a concentration dependent manner. Also, upregulation of EDIL3 protein expression in the whole cell lysates and its secretion in the conditioned medium was detected by immunoblot assay and ELISA, respectively (FIG. 4, panels b, c). Inhibiting TGFβ1 signaling using LY2109761 led to downregulation of EDIL3 expression in both cellular and extracellular compartment along with inhibition of Smad2 activation, establishing the regulation of EDIL3 expression under TGFβ1 control. RNA-sequencing also validated the overexpression of EDIL3 and MFGE8 in CAFs vs NF and indicated only EDIL3 induction under TGFβ1 regulation (FIG. 11 Panels a, b).

The effects of EDIL3 on TGFβ1 induced EMT in CAFs was assessed. EDIL3 was silenced in patient derived CAF using siRNA and validated by RT-PCR with and without TGFβ1 induction (FIG. 4, panel d). Silencing EDIL3 in CAFs led to the downregulation of key EMT marker Transgelin (TGLN) at basal level and significantly abrogated the TGFβ1 induced expression of TGLN and smooth muscle α-actin (ACTA2) in CAFs (FIG. 4, panels e, f). Thus, EDIL3 is overexpressed in CAFs of advanced melanoma patients and modulates TGFβ1 signaling and associated EMT.

EDIL3 correlates with angiogenesis and its signatures in TCGA SKCM and Checkmate 064 databases.

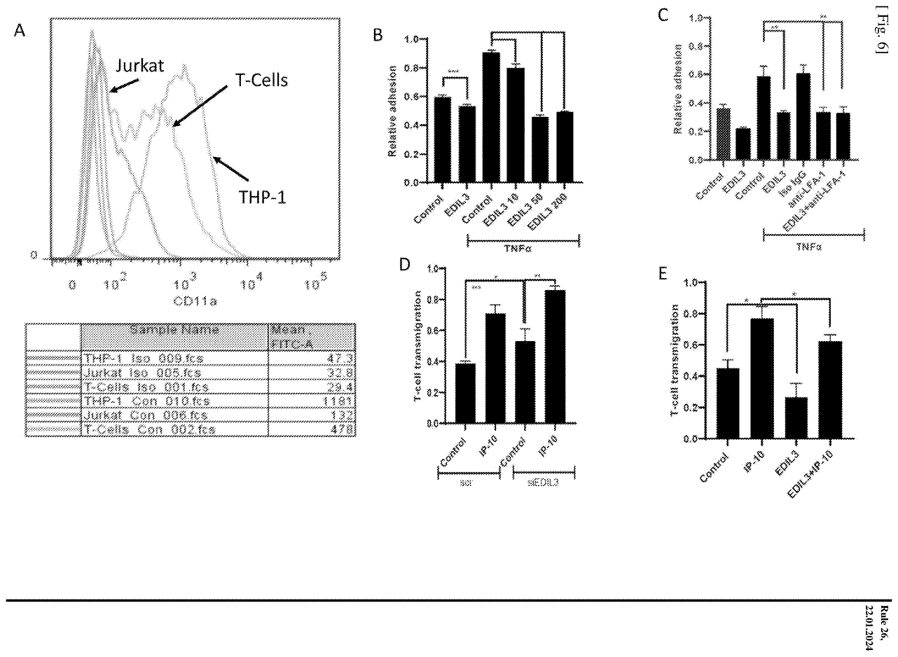

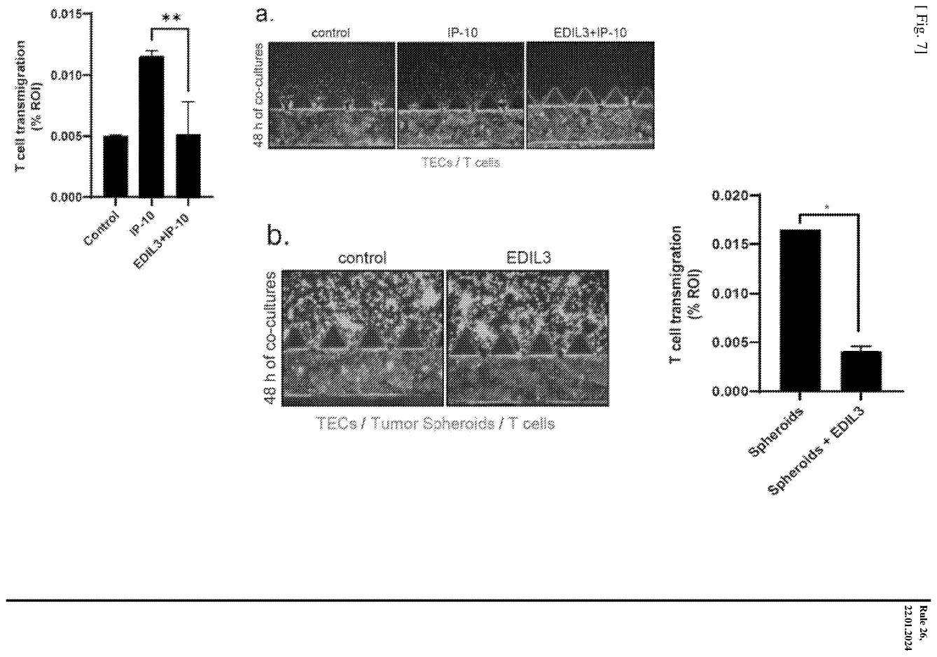

To assess the angiogenic roles of EDIL3, pre-treatment plasma from 39 Ipi-Bev treated patients were analyzed for interdependence of circulating levels of VEGF and EDIL3. The Spearman rank correlation was 0.44 (p=0.005) (FIG. 5, panel a). Thus, a “moderate” relationship was observed indicating that higher pre-treatment EDIL-3 levels was related to higher pre-treatment VEGF levels.

Based on the angiogenetic signature panel (PMID: 29443960 Mariathasan et al) consisting of TEK, CDH5, SOX17 and SOX18, 469 subjects from TCGA SKCM dataset were divided into angiogenesis-high (n=83) and angiogenesis-low (n=386) based on agglomerative clustering with two clusters of the angiogenesis pathway expression. Consistent with our previous analysis, the expression of EDIL3 was significantly upregulated (p=1.3e-07) in patients demonstrating a high angiogenic phenotype vs patient with low angiogenic phenotype. The Checkmate 064 dataset analysis with 90 patients divided as 22 high and 68 low based on angiogenic signature also endorsed the significant relationship (p=0.0002) i.e., EDIL3 is resolutely associated with high angiogenesis in advanced melanoma patients (FIG. 5 panel b).

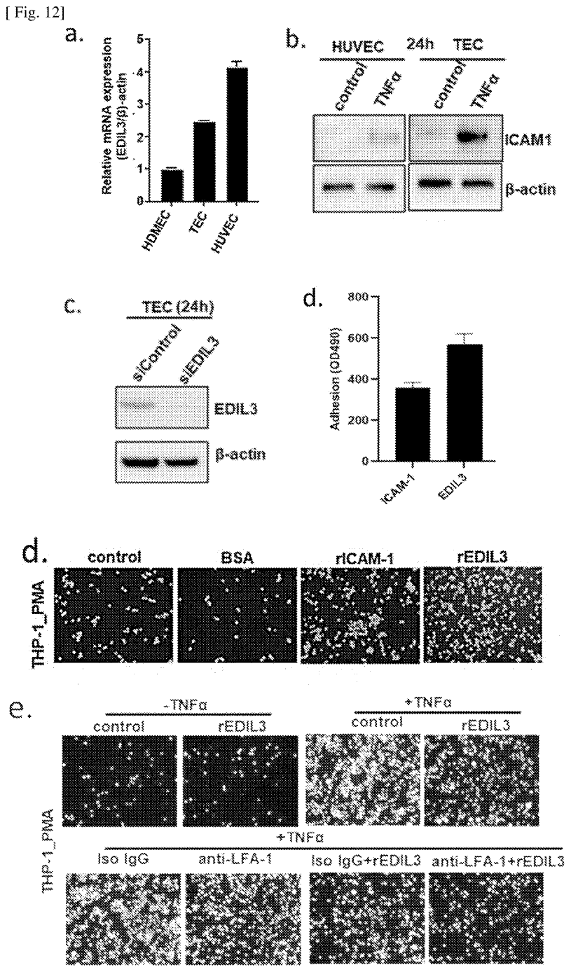

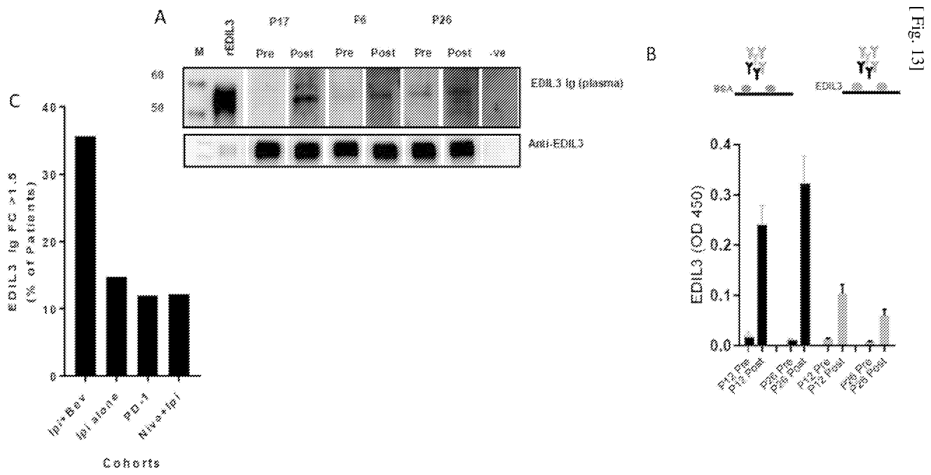

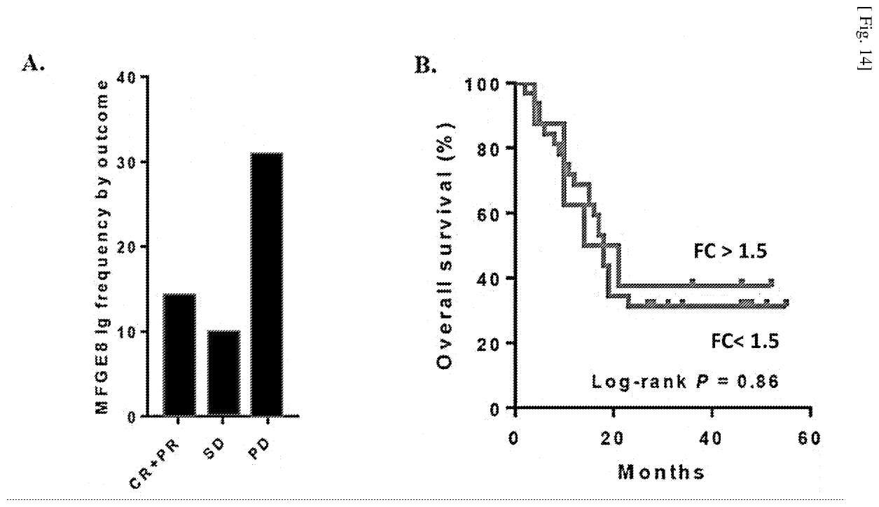

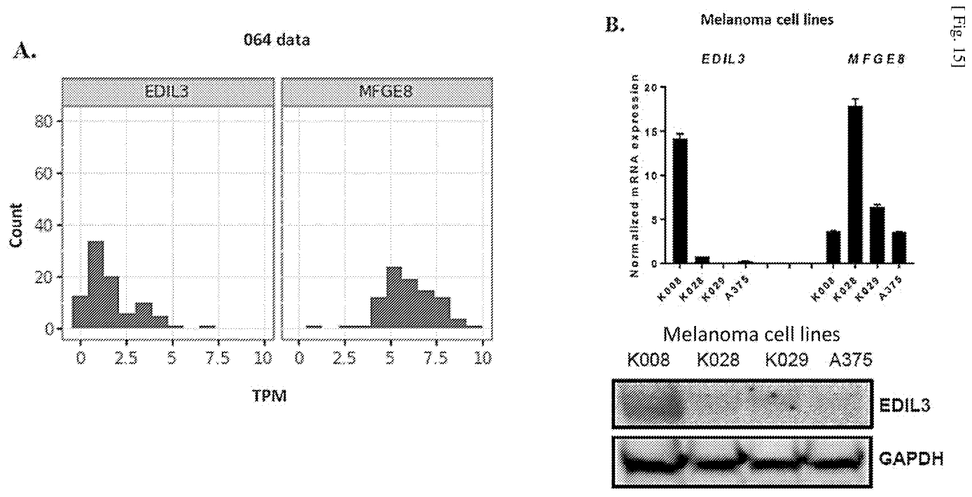

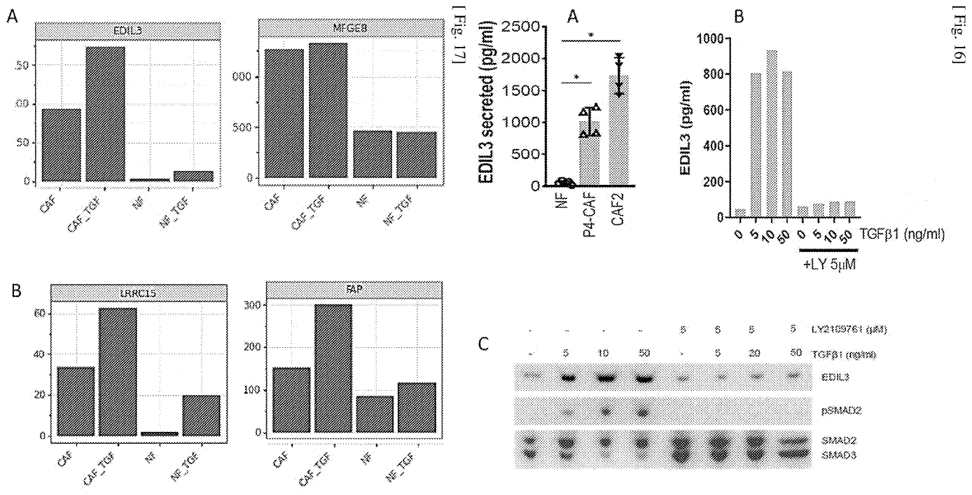

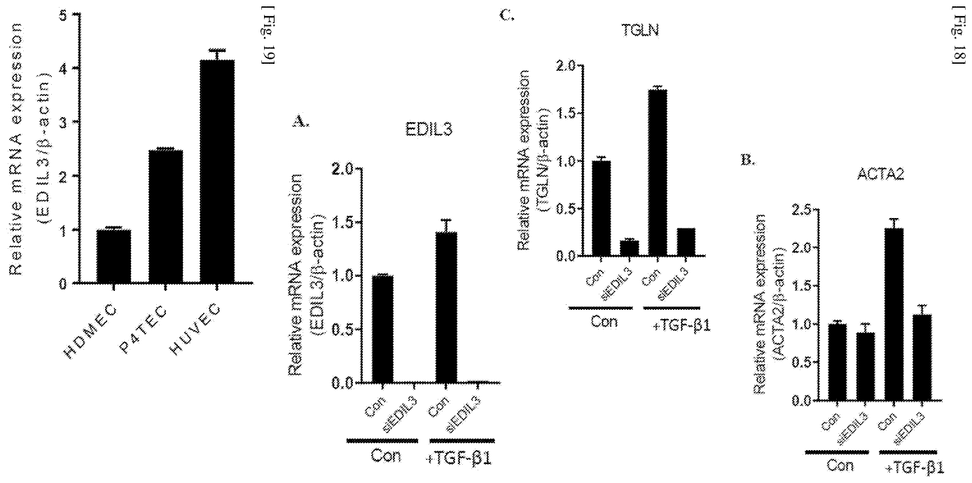

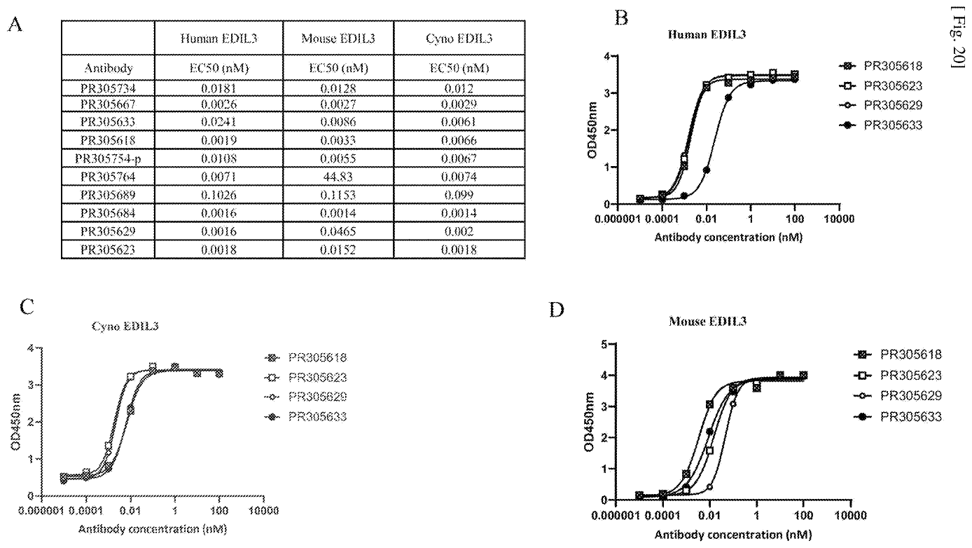

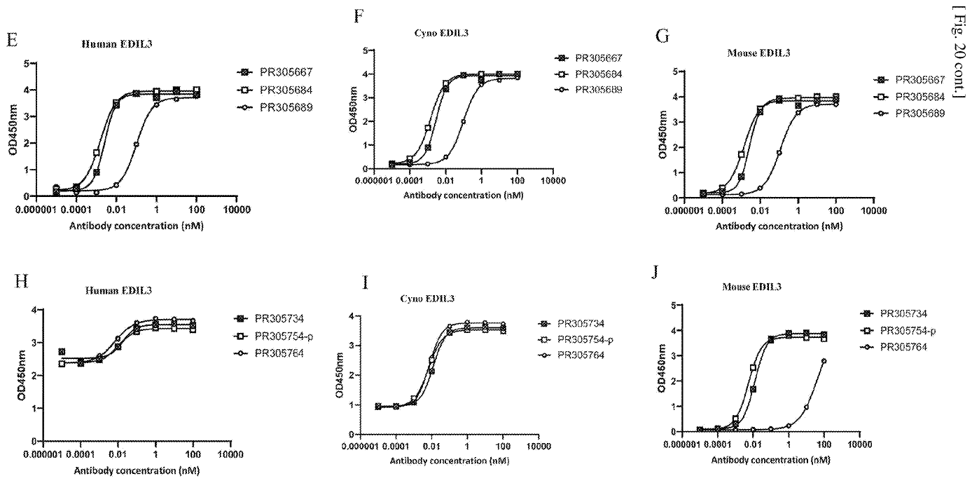

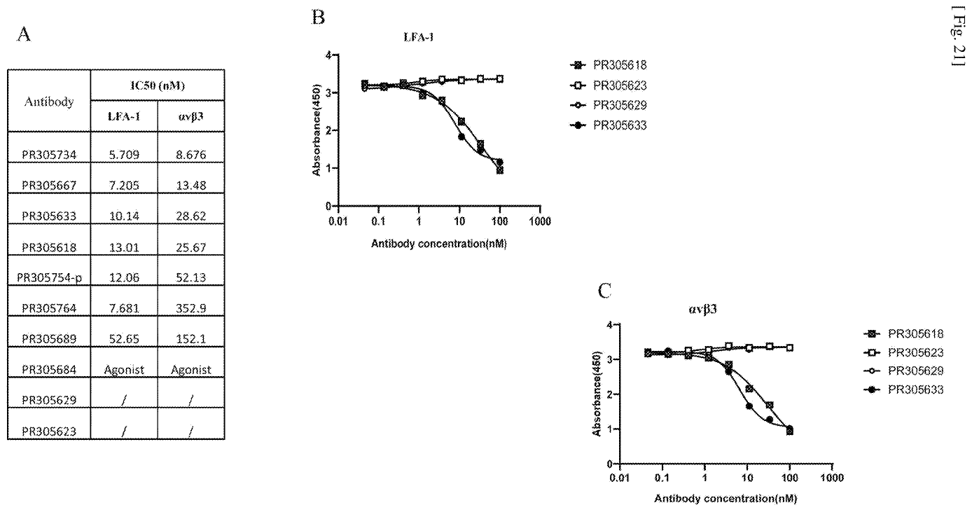

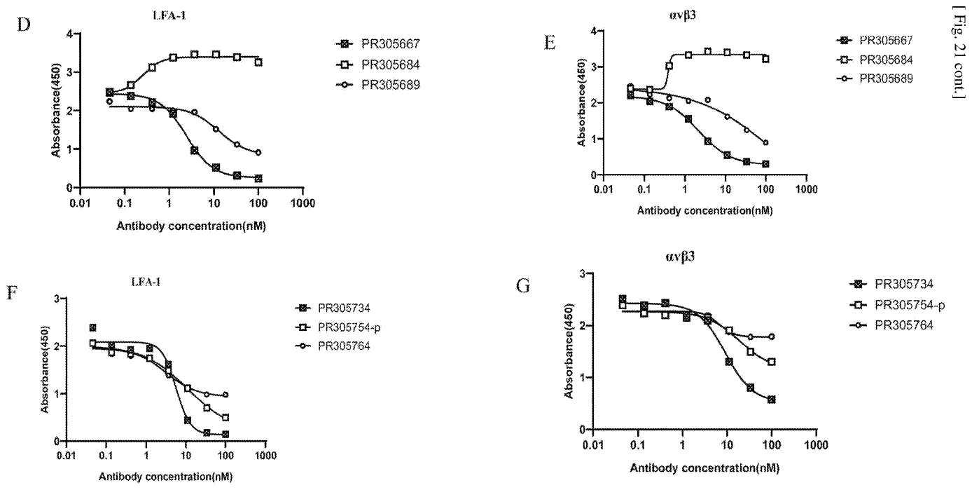

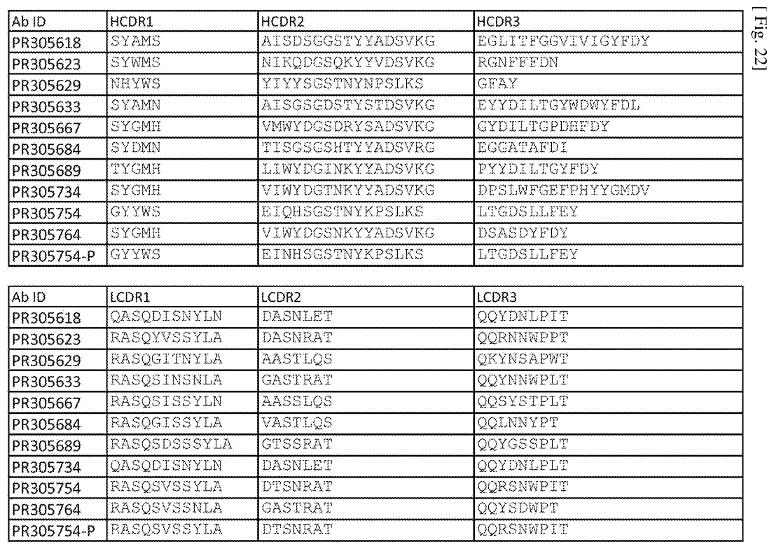

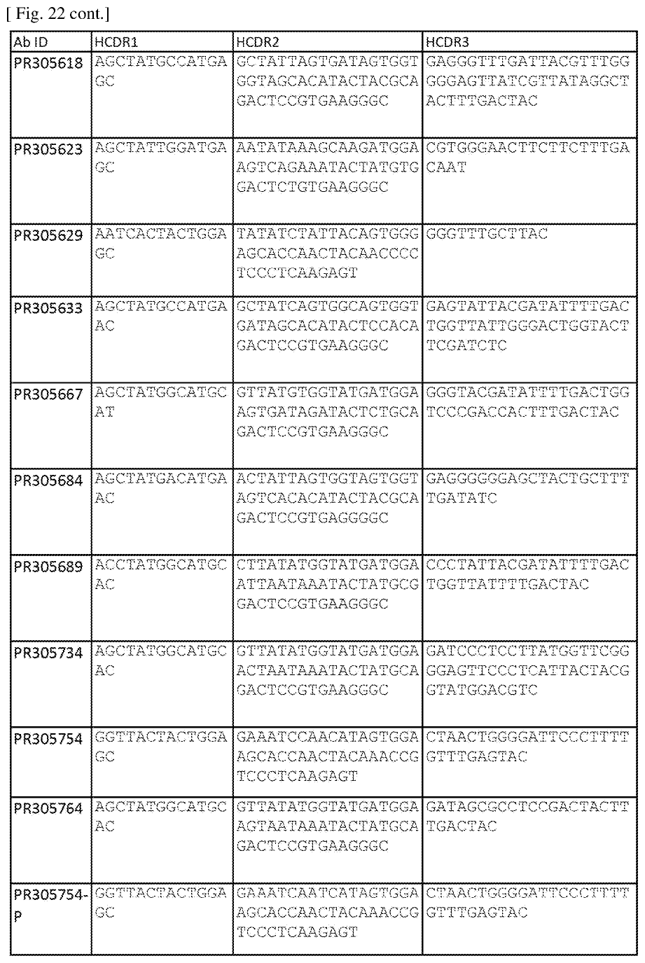

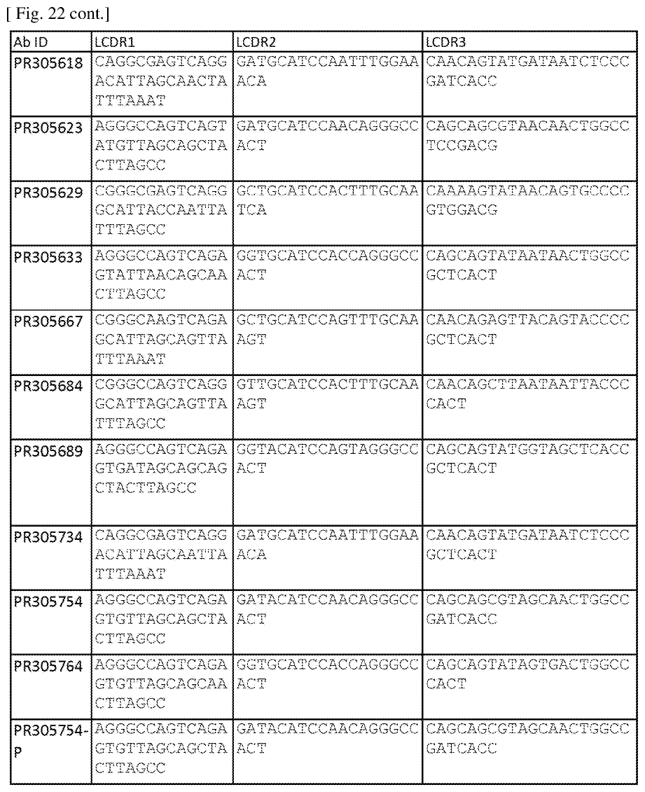

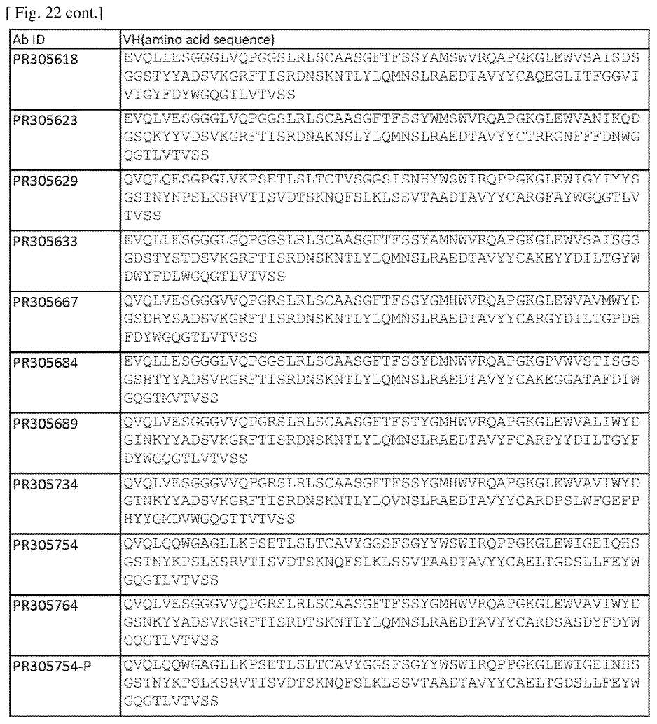

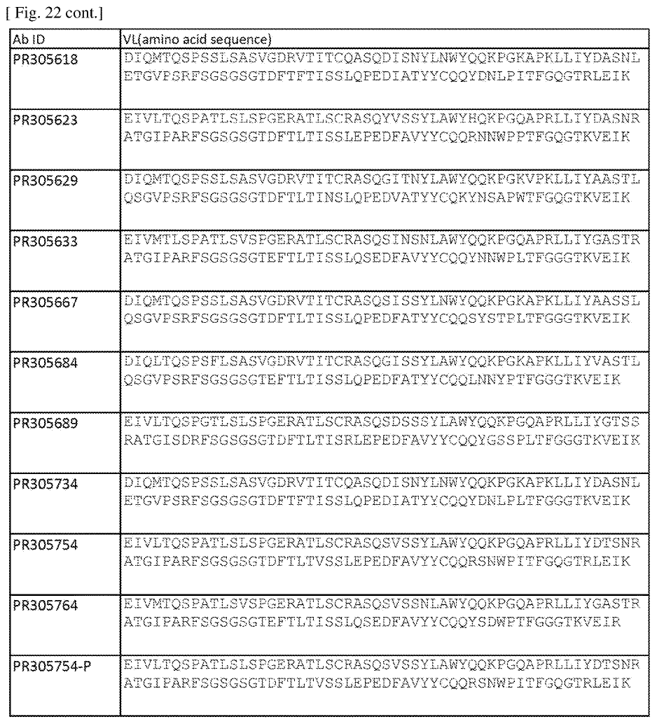

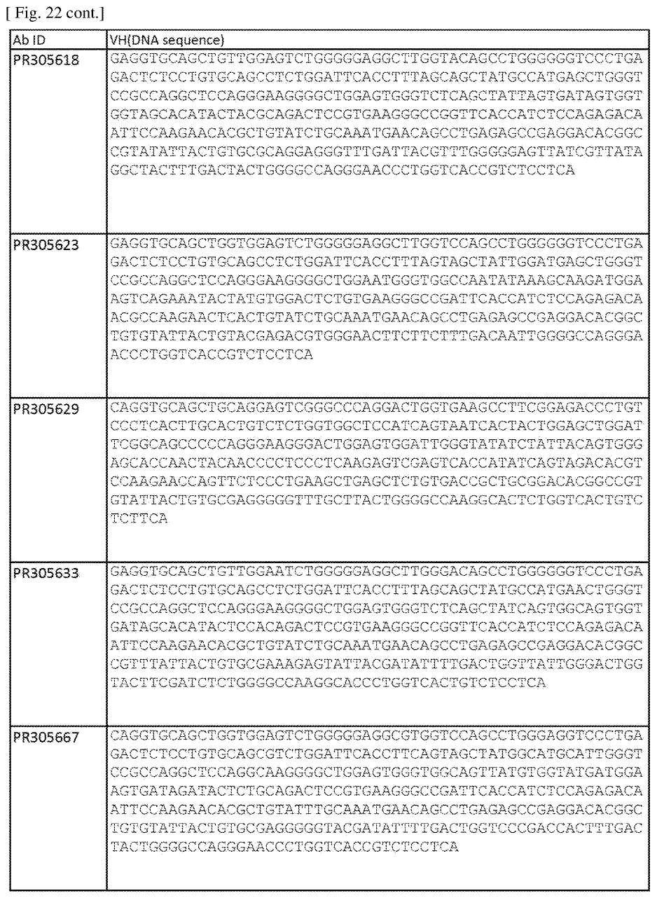

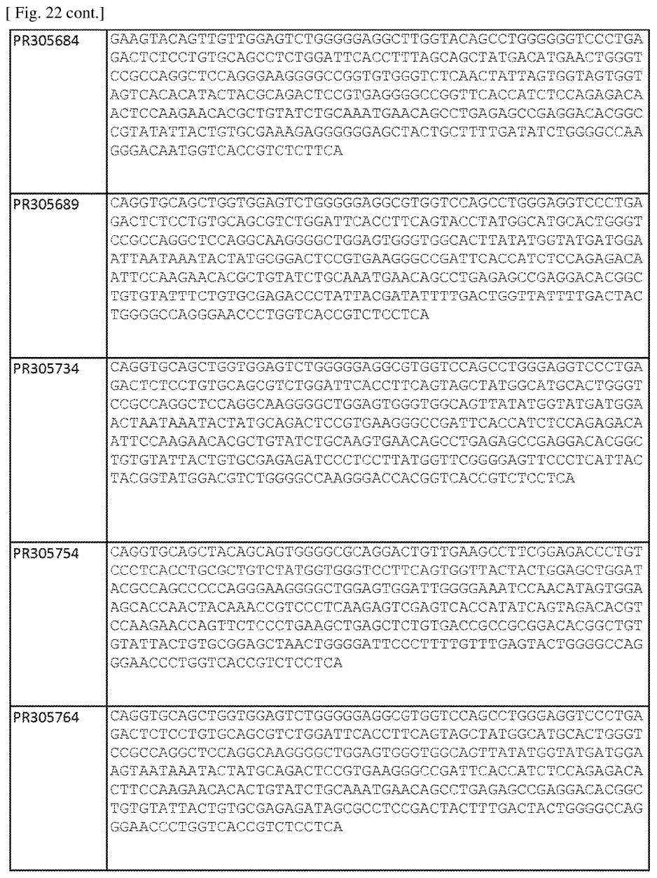

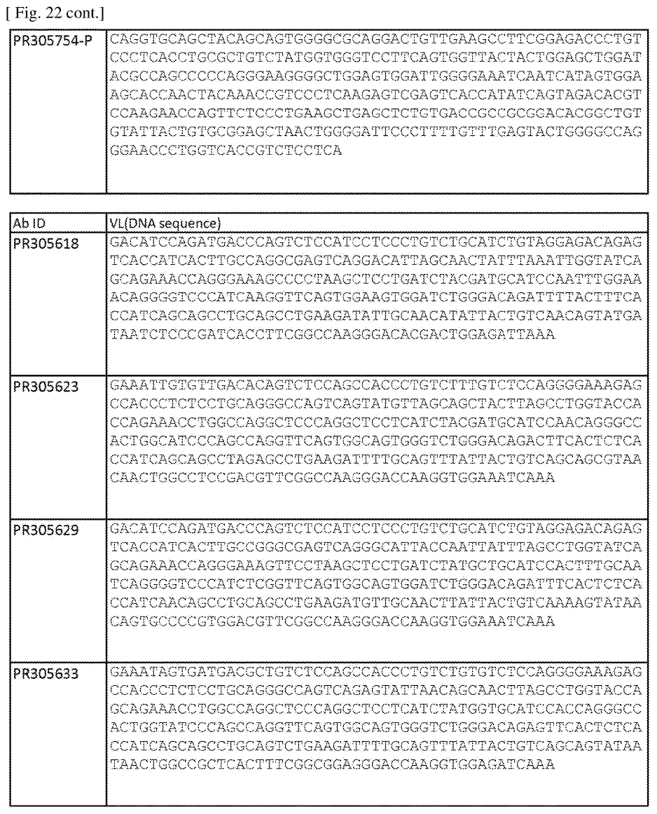

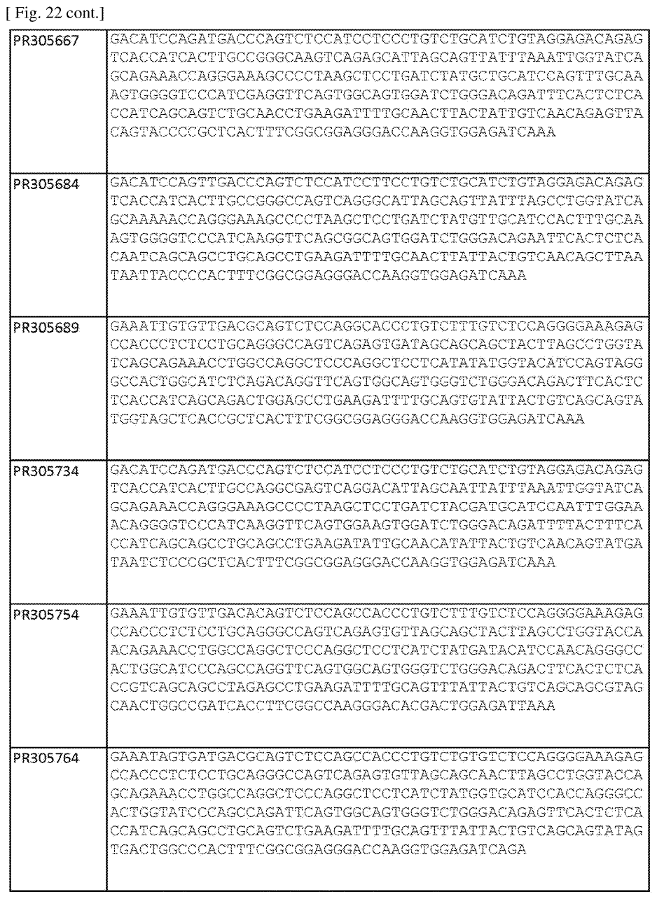

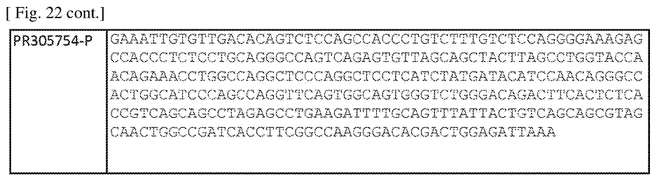

EDIL3 mRNA expression by RT-PCR was found to be increased in endothelial cells with higher angiogenic potential (HMEC, TECs &HUVECs) (FIG. 12 panel a). To further investigate the angiogenic functional effects, recombinant EDIL3 treatment of TECs derived from patients using VEGF A induced effects as positive control was pursued. Recombinant human EDIL3 significantly promoted the migration of TECs vs untreated control and comparable to VEGF A at 24 and 48 hrs (FIG. 5 panel c). It was also found to promote development of more capillary like structures, which were denser compared to the untreated control and like VEGF A treatment (Data not shown) A significant increase in number of branches (mean mesh size, p<0.01) and mesh index (, p<0.05) similar to VEGF was observed with rEDIL3 treatment as compared to control TECs (FIG. 5 panels d, e). Thus, our findings uphold EDIL3 as a positive regulator of pathological angiogenesis.

### EDIL3 Blocks Lymphocyte-Endothelial Adhesion and Inhibits T Cell Migration

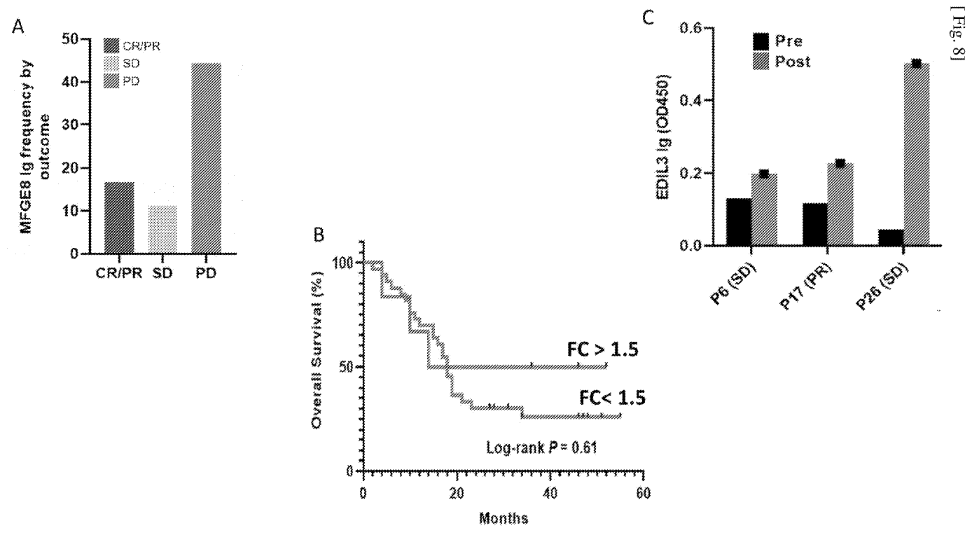

To validate the functional role of EDIL3 in T-cell exclusion in TME, activated immune cells i.e., THP1 cells, Jurkat cells and T-cells were screened for expression of lymphocyte function-associated antigen 1 (LFA-1) by flow cytometric analysis. THP-1 cells were found to be most abundantly expressing LFA-1 among all three cell types screened (FIG. 6, panel a). Adhesion of the immune cells is a pre-requisite for trans-endothelial migration and interaction of LFA-1 on immune cells and ICAM1 (Intercellular Adhesion Molecule 1) on endothelial cells is one of the crucial steps. EDIL3 is known to antagonize the LFA-1/ICAM1 dependent adhesion. To validate the mechanism of T-cell exclusion, adhesion assay was performed, as described above, with Ipi-Bev treated patient derived TECs and THP-1 cells (Data not shown). The patient-derived TEC were left unstimulated or stimulated with TNF-α for 24 h as mentioned in methods. rEDIL3 pre-treatment (for 1 h at 37° C.) of THP-1 cells inhibited their adhesion on unstimulated TEC monolayers as compared to untreated THP-1 cells (p<0.001). TNF-α upregulated expression of ICAM-1 (Data not shown) on TECs leading to significant increase in adhesion of untreated THP-1 cells from 59.6% to 90.2% (p<0.0001) as compared to unstimulated TEC monolayers. However, rEDIL3 pretreatment dose dependently inhibited the adhesion of THP-1 cells on stimulated TEC monolayers i.e., rEDIL3 10 μg/ml by 20% (p<0.001) EDIL3 50 μg/ml and EDIL3 200 μg/ml inhibited comparably by 51.2% (p<0.0001) and 55% (p<0.0001) respectively indicating saturation (FIG. 6, panel b). Next, we confirmed rEDIL3 mediated inhibition of adhesion was mediated by disrupting the LFA-1 and ICAM-1 interaction on TECs. Activated THP1 cells were observed to bind rEDIL3 with higher affinity as compared to rICAM1 in binding assays (Data not shown). THP-1 cells treated with a combination of rEDIL3, and anti-LFA-1 antibody had comparable inhibition of adhesion i.e., 67.8% (p<0.01) on stimulated monolayers of endothelial cells as compared to when treated with rEDIL3 67% (p<0.01) or anti-LFA-1 66% (p<0.01) antibody alone (FIG. 6 panel c). Thus, disruption of LFA-1 and ICAM-1 interaction is one of the underlying mechanisms of EDIL3 mediated inhibition of adhesion of immune cells on TECs.

Next, we studied EDIL3's effect on the trans-endothelial migration of T cells across patient derived TEC monolayer. PBMCs were isolated from donor blood samples by Ficoll-Paque method and T-cells were purified using the Pan-T cell isolation kit. A positive regulator of chemotaxis chemokine IP-10 known to stimulate T-cell migration was used as chemoattractant. The migration of activated T cells across TEC monolayers grown on fibronectin coated microporous membrane of transwells with and without IP-10 in the lower chamber was examined using Boyden chamber assay. IP-10 enhanced the migration of activated T cells when added in the lower chamber as compared to the control. Silencing EDIL3 in TECs increased the transmigration of T-cells in control from 38.5% to 52.85% (p<0.05). The IP-10 induced transmigration was also potentiated following EDIL3 silencing from 52.8% to 85.7% (p<0.01) (FIG. 6, panel d). Meanwhile, rEDIL-3 pretreatment of T-cells decreased the number of transmigrated T cells across the endothelial monolayer in control as well as IP-10 treatment groups (p<0.05) (FIG. 6 panel e).

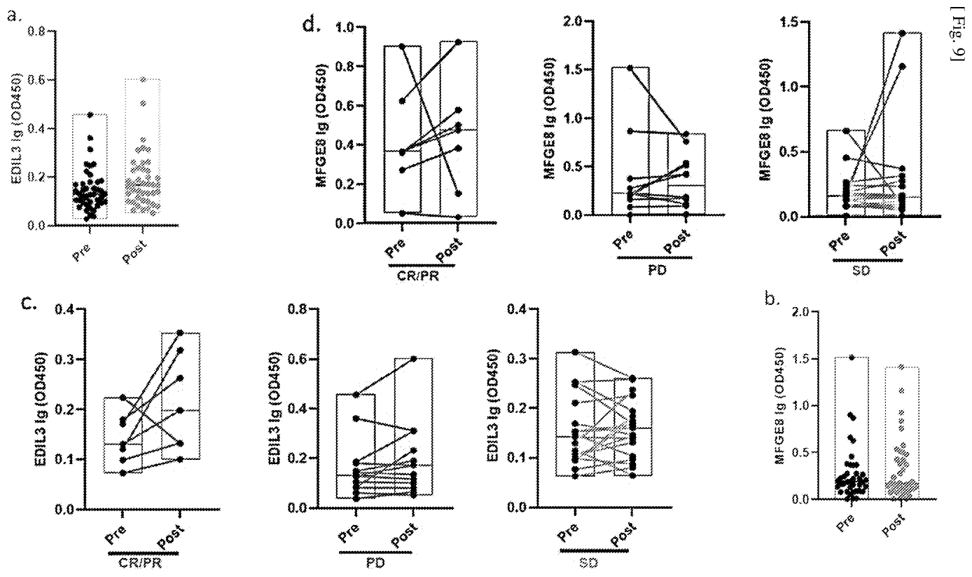

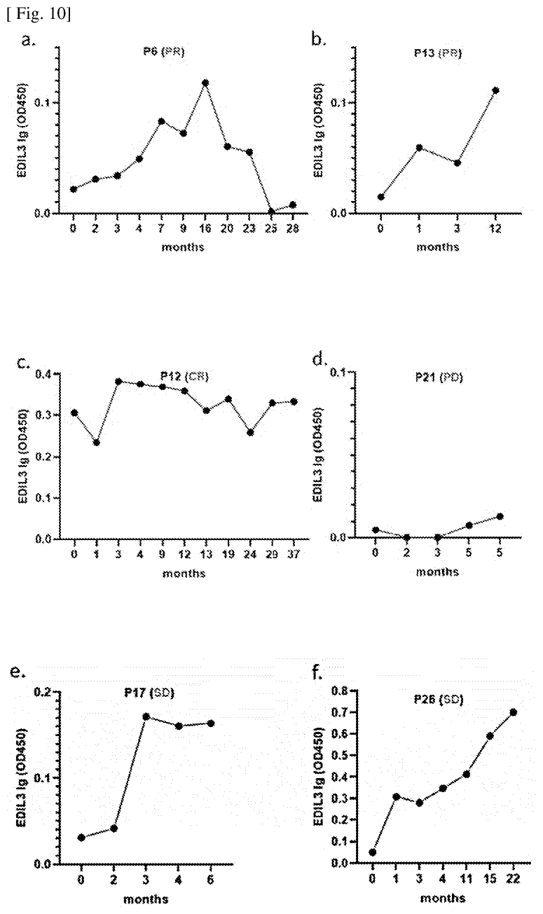

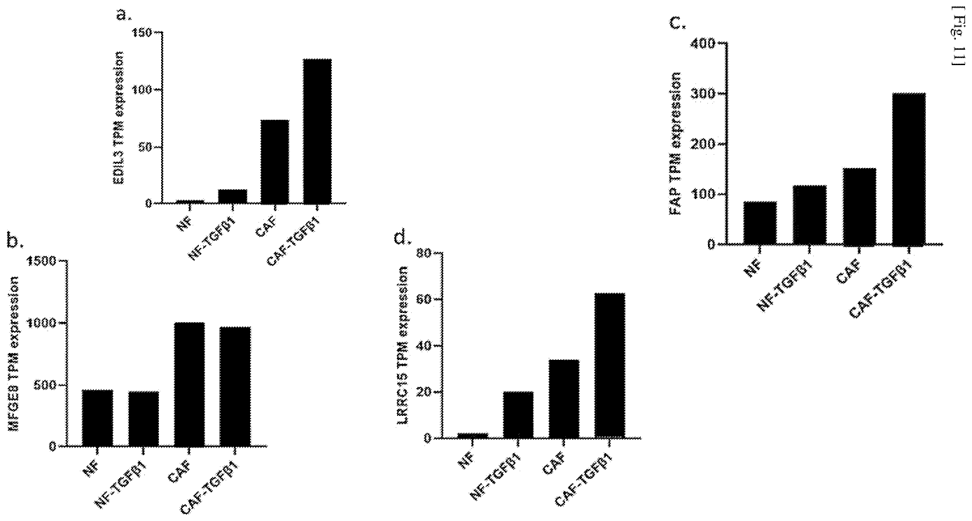

The transmigration of T cells in presence of rEDIL3 was further studied in microfluidic 3D co-cultures. Endothelial vessels were grown using TECs in the DAX-1 3D cell culture chip. Immunofluorescence staining verified the presence of perfusable tubular structures with a clear lumen. The cell-tracker dye was used to determine the positions of T-cells within the chip along the Y-axis. There were two models of co-culture employed to study the transmigration of T-cells across the endothelium. In the first model, 3D vascular model, IP-10 was added in the last chamber while vascular networks were perfused with rEDIL3 pretreated labelled T cells and allowed to transmigrate over 48 h. Once significant transmigration was observed for control vs IP-10 treatment (p<0.001) the ROIs were selected, imaged, and quantified. While IP-10 induced the transmigration by 2.3-fold, the addition of EDIL3 pretreated T-cells and rEDIL3 in media significantly decreased the overall IP-10 induced migration (p<0.01) of T-cells (FIG. 7, panel a).

In the second model, the Tumor-vascular model, along with the vascular networks on one side of the chip, ECM containing tumor spheroids was added in middle chamber as source of IP-10 to establish a chemotactic gradient to facilitate the T cell transmigration. The vascular networks were perfused with untreated or rEDIL3 pretreated T cells, while maintaining rEDIL3 in the media replenished at the vascular port and co-cultured for 48 h. Next, the T cells extravasation from the TECs vessels was determined. Quantification of the fluorescent T cells in the middle compartment among the spheroids was performed. The EDIL3 pretreatment significantly (p<0.05) downregulated the transmigration of T-cells across the vascular networks (FIG. 7, panel b). Thus, one of the mechanisms by which EDIL3 inhibited T cell migration can be by blocking the LFA-1 on T-cells and ICAM1 on TECs interaction vital for adhesion, thus resulting in exclusion of T-cells in TME.

### Discussion

The combination of Ipi-Bev therapy elicited a functional humoral immune response towards EDIL3 which was associated with clinical benefit in advanced melanoma patients. Interestingly, humoral responses against MFGE8 (milk fat globule—EGF factor 8), another secreted glycoprotein similar to EDIL3 was also observed. EDIL3 and MFGE8 are evolutionary related and share sequence similarities (24). They are homologous secretory glycoproteins that possess an evolutionally conserved RGD motif. These glycoproteins are implicated in the regulation of leukocyte recruitment and inflammation (4). However, their roles in tumor immunology and therapeutics are limited. Though, anti-MFGE8 responses alone were not associated with improved clinical outcomes, systemic targeting of MFGE8 strengthens cross-presentation of immunogenic antigens (b). Thus, anti-MFGE8 response can contribute to tumor immunogenicity observed in patients treated with Ipi-Bev combination. Out of the cohorts screened, EDIL3 antibody response was most robust in patients treated with combination of Ipi-Bev as compared to Ipi alone, anti-PD1 and combination of Ipi-Nivo. Bevacizumab in combination with IO is reported to increase CD8 T lymphocyte infiltration in melanoma tumors (a), while EDIL3 was found to be associated with immune exclusion. Thus, EDIL3 can be an immunomodulatory target of bevacizumab and can serve as biomarker of response toward Ipi-Bev combination therapy.

Our TIDE correlatives delineated the positive association of EDIL3 expression and gene signatures of immune evasion through T cell exclusion in large melanoma patient cohorts. High EDIL3 expression in SKCM subjects predicted non-response to ICB therapy consistent with previous reports of EDIL3 as a poor prognostic marker in multiple malignancies (5, 6). Our in vitro adhesion and trans-endothelial migration studies validated recombinant EDIL3 mediated T cell exclusion at the vascular-immune interface. EDIL3 acts as an endogenous inhibitor of LFA-1-dependent leukocyte recruitment in inflammatory diseased conditions (1). The current findings can be important for understanding the robust CD8+ T cells infiltration seen in patients receiving benefit to treatment while developing humoral immunity to EDIL3. EDIL3 is an important player in T cell immunity via promoting Treg responses in inflammatory conditions by upregulating the FOXP3 expression via αᵥβ₃ integrin signaling (2).

Among the cell types known to mediate T cell exclusion, CAFs were found to associate with EDIL3 expression. There are reports exploring the role of EDIL3 in fibroblasts emphasizing the current findings. A colorectal carcinoma liquid biopsy analysis traced EDIL3 as a plasma-derived exosomal cargo protein exclusively associated with patient-derived CAFs (4). CAFs secreted EDIL3 has been indicated as a prognostic signature associated with clinical outcome, tumor progression, and genetic alteration in breast cancer (3). In line with the TCGA analysis, in our in vitro studies TGFβ1 induced the expression of EDIL3, and its silencing inhibited the TGFβ1-induced EMT phenotype in CAFs. Indicating an EDIL3-mediated molecular feedback loop in regulating TGFβ-induced EMT in CAFs (7). Mariathasan et al. reported TGFβ signaling in fibroblasts to be associated with a lack of response towards ICB, particularly in tumors showing CD8⁺ T cell exclusion. Thus, the humoral response against EDIL3 emphasizes the importance of normalizing the immune-vascular crosstalk to unleash anti-tumor immunity.

In conclusion, EDIL3 produced by CAFs supports immune evasion through T cell exclusion and regulation of TGF-β induced EMT in melanoma tumors. Without wishing to be bound by theory, EDIL3 can be one of the underlying targets of bevacizumab effectuating an excluded to an inflamed phenotype observed in responders of Ipi-Bev treatment. Without wishing to be bound by theory EDIL3 not only provides useful mechanistic insight into antitumor and synergistic effects of combining ICB and anti-VEGF agents, but further establishes as a therapeutic target.

### Example 2

### Antibody Production and Purification

Harbour H2L2 Mice® were immunized with human EDIL3-Fc or EDIL3-his (from novoprotein) to generate anti-EDIL3 antibodies. Mice with high serum titers and specific immune responses against Human EDIL3-his, mouse EDIL3-his and cyno EDIL3-his were selected and final boosted with Human EDIL3-Fc or EDIL3-his protein three days before being sacrificed for spleen, bone marrow and lymph node harvest. A nanofluidic optoelectronic B lymphocyte screening technique (NanOBlast) was used for this study. The NanOBlast workflow begins with preparation of antibody secreting cells (ASCs). Selectively enriched, antigen-experienced murine ASCs were isolated from lymphocytes harvested from spleen and lymph nodes with mouse CD138 antibody coated magnetic beads. ASCs were imported into the 14 k size chip and sequestered into individual nanopens via OptoElectro Positioning (OEP). ASCs that secrete antigen-specific IgG are detected using protein-based fluorescent binding assay that produces a characteristic fluorescent bloom (Human EDIL3-his, mouse EDIL3-his and cyno EDIL3-his protein for screening). Individual ASC cells of interest were then un-penned using OEP and exported from the chip directly into 96-well plates containing cell lysis buffer. Antibody heavy chain variable domain (VH) and light chain variable domain (VL) sequences were recovered using single cell rapid amplification of cDNA ends (RACE), cloned and recombinantly expressed as canonical antibodies using standard methods.

After obtaining the sequences encoding the light and heavy chain variable region of each screened antibody molecule, the nucleic acid sequences encoding the light and heavy chain variable region of each antibody molecule were fused with the nucleic acid sequences encoding the light and heavy chain constant domains of the human antibody and expressed by recombinant DNA techniques to obtain recombinant antibody molecules. The nucleic acid sequence encoding the heavy chain variable region (VH) of the antibody was genetically synthesized and cloned into a mammalian cell expression plasmid vector (pTT5 mammalian expression vector) comprising the nucleic acid sequence encoding the heavy chain constant domain of the human IgG1 antibody so as to encode a full-length heavy chain. The nucleic acid sequence encoding the light chain variable domain (VL) of the antibody was genetically synthesized and cloned into a mammalian cell expression plasmid vector (pTT5 mammalian expression vector) comprising the nucleic acid sequence encoding the light chain constant region of the human Igκ antibody so as to encode a full-length light chain.

The plasmid encoding the heavy chain of the antibody and the plasmid encoding the light chain of the antibody were simultaneously transfected into human embryonic kidney cell HEK293. Purified recombinant anti-EDIL3 antibody with light and heavy chain correctly assembled in pairs can be obtained from these HEK293 cells by conventional recombinant protein expression and purification techniques.

HEK293 cells were expanded in FreeStyle™ F17 Expression Medium (Thermo, #A1383504). Before transient transfection, the cells were adjusted to a concentration of 6-8×10⁵ cells/mL and cultured in a shaker at 37° C. with 8% CO2 for 24 h to a concentration of about 1.2×10⁶ cells/mL. 30 mL of cultured cells were taken. The plasmid comprising the nucleic acid sequence encoding the heavy chain of the antibody and the plasmid comprising the nucleic acid sequence encoding the light chain of the antibody described above were mixed in a ratio of 2:3, with a total of 30 μg of plasmids dissolved in 1.5 mL of Opti-MEM reduced serum medium (Thermo, 31985088), and the medium was filtered through a 0.22 μm filter for sterilization. Then, 1.5 mL of Opti-MEM was mixed with 120 μL of 1 mg/mL PEI (Polysciences, Inc. #23966-2), and left to stand for 5 min. PEI was slowly added to the plasmid, and incubated at room temperature for 10 min. The mixed solution of plasmid and PEI was slowly dropped into the culture flask while shaking and cultured in a shaker at 37° C. with 8% CO2 for 5 days. Cell viability was measured after 5 days. The culture was collected and centrifuged at 3300 g for 10 min, and then the supernatant was collected and centrifuged at high speed to remove impurities. A gravity column (Bio-Rad, #7311550) containing MabSelect™ (GE Healthcare Life Science, #71-5020-91 AE) was equilibrated with PBS (pH 7.4) and rinsed with 2-5 column volumes of PBS. The column was loaded with the supernatant sample and rinsed with 5-10 column volumes of PBS, followed by 0.1 M glycine at pH 3.5 to elute the target protein. The eluate was adjusted to neutrality with Tris-HCl at pH 8.0 and concentrated and buffer exchanged into PBS buffer with an ultrafiltration tube (Millipore, UFC901024) to obtain a purified solution of anti-EDIL3 antibody. Finally, the concentration of purified solution was determined using NanoDrop (Thermo Scientific™ NanoDrop™ One), and the purified solution of anti-EDIL3 antibody was divided into aliquots and stored for later use.

Detection of antibody binding activity to EDIL3 protein.

The binding activity to the EDIL3 protein of 10 antibodies obtained using the protocol described above were determined using ELISA assay. Human EDIL3 (novoprotein, #C601), cyno EDIL3 (novoprotein, #C18M) and mouse EDIL3 (novoprotein, #C18P) proteins were diluted to a concentration of 1 μg/mL, added to a 96-well plate (100 μL per well) and placed at 4° C. overnight. The 96-well plates were washed three times with PBST solution and then placed at 37° C. for 1 h with the addition of 2% BSA in PBS solution. Antibody series dilutions were prepared (starting from 100 nM, 1:10 dilution, 8 points), added to a 96-well plate, and incubated at 37° C. for 1 h. After washing the wells three times with PBST solution, 100 μL of an anti-human IgG Fc-HRP secondary antibody solution was added per well (for 5000× dilution) and incubated at 37° C. for 50-60 min. After washing the wells three times with PBST solution, a TMB color development solution was added and after 5-15 minutes, a termination solution was added to stop the reaction.

The test results are shown in FIGS. 20A-J. Antibodies PR305734, PR305667, PR305633, PR305618, PR305754-p, PR305764, PR305689, PR305684, PR305629 and PR305623 showed single to double digits picomolar EC50 binding activity to human EDIL3 protein, to Cyno EDIL3 protein and to mouse EDIL3 protein.

Blockade Assay of Anti-Human EDIL3 Monoclonal Antibody to the Binding of Human EDIL3 with Ligand LFA-1 Protein and αvβ3 Protein by ELISA

The blocking activity of anti-human EDIL3 monoclonal antibody to the binding of human EDIL3 (Novoprotein, C601-1 mg) to ligand LFA-1 protein (ACRO, IT2-H53W3-250 ug) and ligand αvβ3 protein (ACRO, IT3-H52E3-500 ug) was assessed. The EDIL3 protein was biotinylated using a biotinylation kit (ThermoFisher, A39257, EZ-Link Sulfo-NHS-LC-Biotin) according to the instructions. 96-well plates (Corning, Cat #: 9018) were coated with 2 μg/mL of LFA-1 protein (ACRO, IT2-H53W3-250 ug) or αvβ3 protein (ACRO, IT3-H52E3-500 ug) overnight, then washed three times with PBST solution and then placed at 37° C. for 1 h with the addition of 200 μL per well 2% BSA in PBS solution. The plates were washed 3 times with PBST and 50 μL of a test antibody gradient dilution (starting from 100 nM, 1:3 dilution, 8 points) and 50 μL EDIL3-his-biotin (final concentration: 2 μg/ml) were added per well. After the plates were incubated at room temperature for 1 h, the supernatant was discarded. Then 100 ul of 1:5000 dilution of Streptavidin-coupled HRP (Sigma, Cat #: S2438) was added per well. The plates were incubated for 1 h at 37° C. and washed 3 times with PBST. 100 μL of TMB was added per well for color development, and the reaction was stopped 15 min later by the addition of 50 μL of stop solution. OD450 values were measured using Spetra max 384 plus (Molecular device)

As shown in the FIGS. 21A-G, PR305618, PR305633, PR305667 and PR305734 are the antibodies with the best blocking activity. The specific IC50 values are shown in the FIG. 21A.

EDIL3 mAbs Binds Specifically to Human Recombinant EDIL3

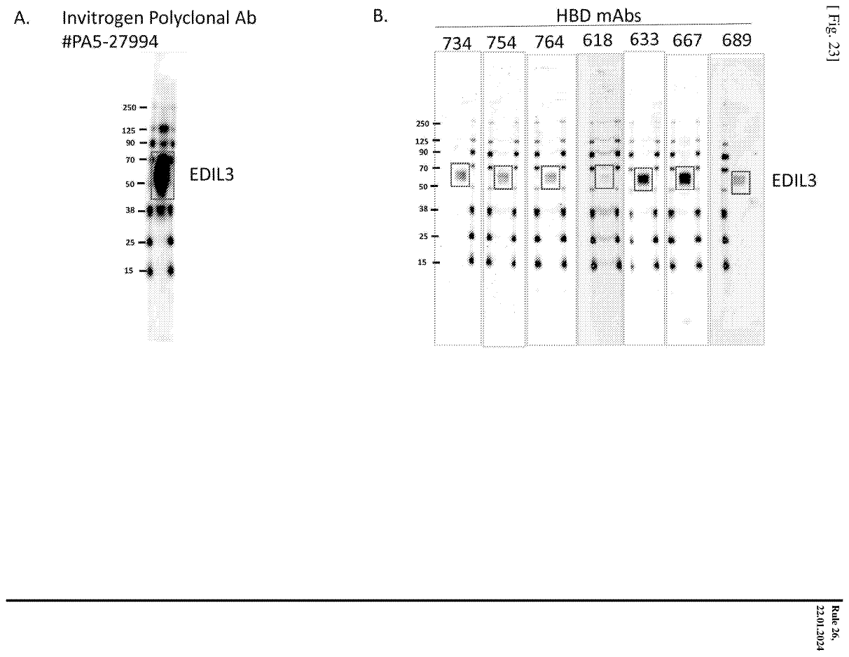

The binding of EDIL3 mAbs to human recombinant EDIL3 was performed by Western blot analysis and conditioned medium by 10% reducing SDS-PAGE. Human recombinant EDIL3 were transferred to a membrane and probed with a EDIL3 Polyclonal Antibody (Product #PA5-27994) or EDIL3 mAbs PR305734, PR305754, PR305764, PR305618, PR305633, PR305667, PR305689 at a ratio dilution. The HRP-conjugated anti-rabbit IgG antibody was used to detect the primary antibody. As shown in the FIGS. 23A-B, the EDIL3 protein is about 65 kDa predicted band in reducing conditions, the EDIL3 mAbs PR305734, PR305754, PR305764, PR305618, PR305633, PR305667, PR305689 can bing to the human recombinant EDIL3, like the EDIL3 Polyclonal Antibody (Product #PA5-27994).

Determination of Kinetics and Thermodynamic Constants for EDIL3 mAbs by Surface Plasma Resonance

Surface Plasmon Resonance (SPR) as known in the art was used to determine the binding affinities of EDIL3 antibodies PR305734, PR305754, PR305764, PR305633, PR305667, PR305618, PR305689. The SPR-based binding method involves immobilization of a ligand (antibody) on the surface of a sensor chip. The binding partner of interest or an analyte flow through the flow channel. Different concentrations of an analyte flow over the ligand, and the interactions of ligand-analyte can be characterized. The SPR signal originates from changes in the refractive index of the light source at the surface of the sensor chip. The increase in mass associated with a binding event causes a proportional increase in the refractive index, which is observed as a change in response-resonance signal.

In brief, for the experiments using the antibodies described herein, the SPR assay was carried out by immobilizing antibodies (used as analyte) at a concentration ranging from 1-1000 nM and flow-through EDIL3 antigen (used as ligand). The antibody kinetic data for the interaction with EDIL3 antigen were fitted to the 2-state and 1-1 binding models using Biocore software. The mean and standard deviation KD values are derived from at least three independent runs.

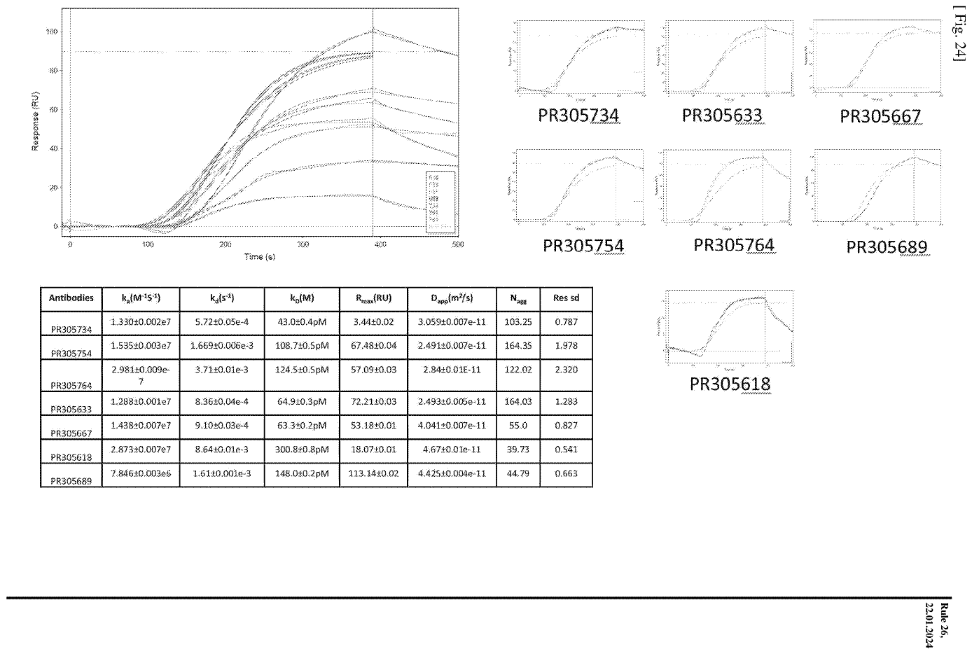

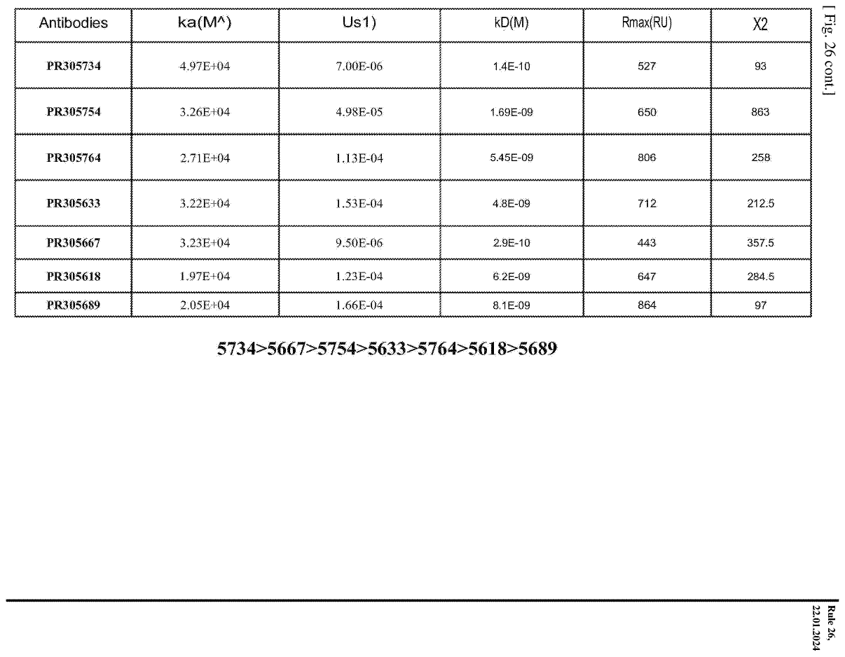

FIG. 24 and FIG. 26 shows the kinetics measurements for 7 EDIL3 antibodies to bind to EDIL3. All seven EDIL3 antibodies show nM or sub-nM affinities for EDIL3 and slow off-rate kinetics (~1×10-5-6 min-1) with high goodness of fits. Rankings agree well with our previous assessments of kinetics using other SPR instruments.

Studying the Blocking Activity of EDIL3 mAbs

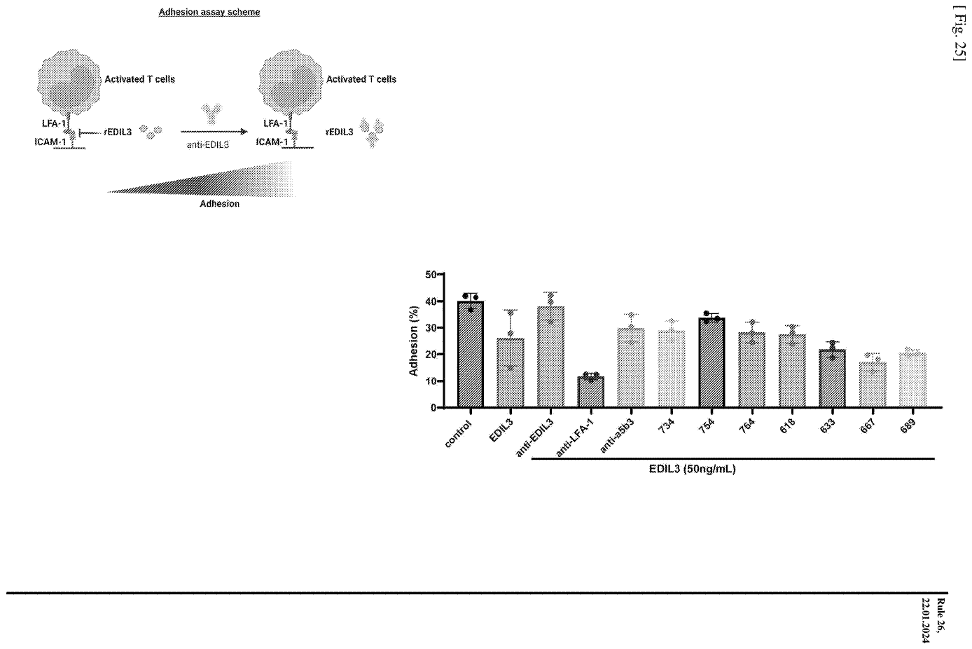

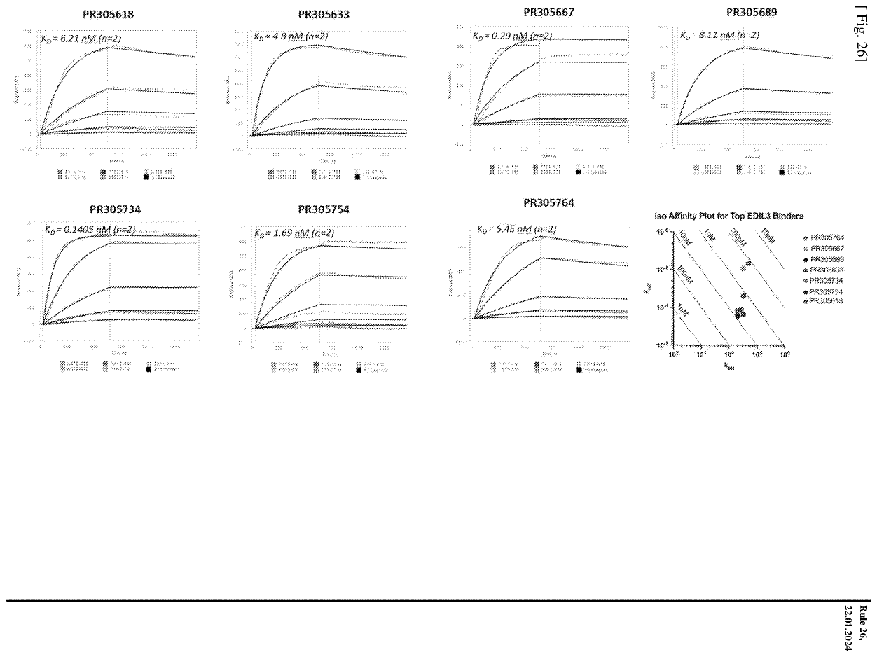

Adhesion assay scheme were performed as described in FIG. 25. Briefly, TECs were grown on fibronectin-coated 96- or 24-well black-walled plates. After 24 hours of seeding, the cells were treated with BSA or stimulated with TNFa (BioLegend; #570102; 5 ng/mL) for another 24 hours. Activated T cells were labeled with BCECF (1 mmol/L, Life Technologies; #B1170) for 30 minutes, followed by incubation with the indicated concentrations of rEDIL3 (50 ng/ml) for 1 hour at 37_C in dark or low light conditions. Endothelial monolayers were washed three times with PBS at the end of the treatment. Pretreated BCECF-labeled activated T cells were added to all the wells and allowed to adhere for 45 minutes at 37_C. Then add a certain concentration of EDIL3, anti-EDIL3, anti-LFA-1, anti-a5b3, EDIL3 antibodies PR305734, PR305754, PR305764, PR305618, PR305633, PR305667, PR305689

Fluorescence intensity was measured at 485 nm (excitation) and 530 nm (emission), before (input), and after four washes with warm RPMI1640 medium to remove nonadherent cells using a SpectraMax M3 Multi-Mode Microplate Reader. The adhesion data were plotted as a percentage of the untreated control (100%). Representative images were acquired for each treatment group. The FIG. 25 shows the activity of EDIL3 mAbs blocking the binding LFA-1 to ICAM-1.

### Epitope Binning Analysis of EDIL3 Antibodies

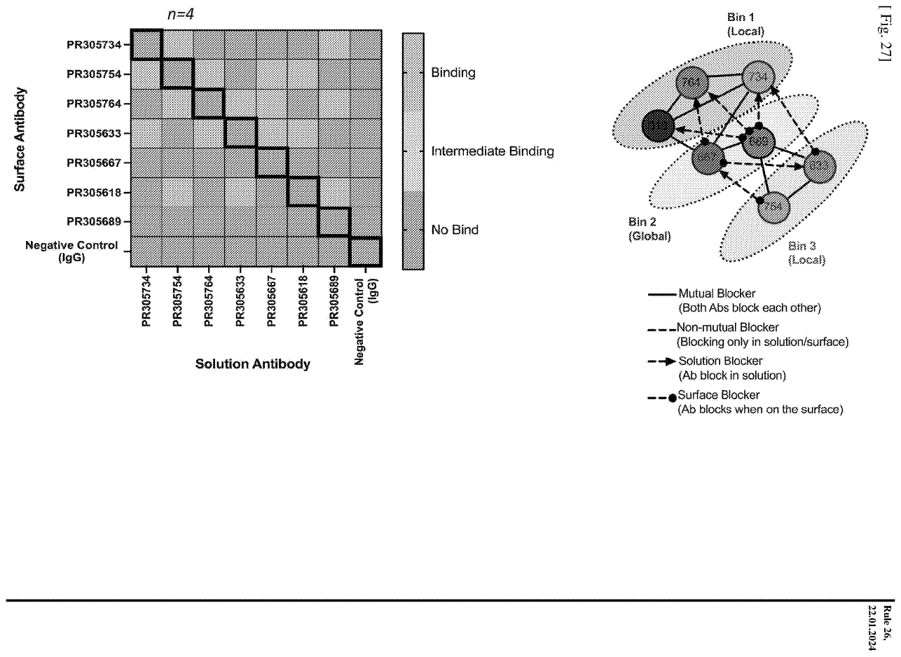

To determine if the EDIL3 antibodies bind to EDIL3 on different or approximate binding epitopes, epitope competition experiments for the EDIL3 antibodies were performed by the ForteBio Octet® RED96e platform. EDIL3 protein was diluted to 3 μg/ml with the kinetic buffer (10× kinetic buffer (Catalog #18-1105, ForteBio)), and then loaded onto anti-Penta HIS biosensors (Catalog #18-5120, ForteBio) to reach capture levels to 0.3 nm. The in-tandem competition assay format was applied, and it contained two association steps. Firstly, the antigen-loaded biosensors bound to each antibody (i.e., First antibody, 1st Ab) at a saturating concentration of 400 nM for 300 seconds to reach equilibrium and then secondly bound to the competing antibodies (i.e., Second antibody, 2nd Ab) of 400 nM for 300 seconds. The second binding signals are recorded as the 100% signal of each antibody when the first antibodies are replaced by kinetic buffer. All the binding data were analyzed using ForteBio Data Analysis 11.0 software. The results in FIG. 27 show that the seven EDIL3 antibodies PR305734, PR305754, PR305764, PR305618, PR305633, PR305667, PR305689 bind overlapping yet distinct epitopes on EDIL3 that is dependent on antibody presentation (i.e. solution vs. surface presentation). None of these antibodies show interact with EDIL3 in the presence of themselves (i.e. show biparatopic activity) or interact with EDIL3 in the presence of the negative control

### EQUIVALENTS

Those skilled in the art will recognize, or be able to ascertain, using no more than routine experimentation, numerous equivalents to the specific substances and procedures described herein. Such equivalents are considered to be within the scope of this invention, and are covered by the following claims.

1. An antibody or antigen-binding fragment or variant thereof that specifically binds epidermal growth factor-like repeats and discoidin I-like domains 3 (EDIL3) protein, wherein the antibody or antigen-binding fragment comprises a heavy chain variable region (HCVR) and a light chain variable region (LCVR), wherein the HCVR comprises the complementarity determining regions (CDRs) HCDR1, HCDR2 and HCDR3 and the LCVR comprises CDRs LCDR1, LCDR2 and LCDR3, wherein:
a) the amino acid sequence of HCDR1 is SYAMS, the amino acid sequence of HCDR2 is AISDSGGSTYYADSVKG, the amino acid sequence of HCDR3 is EGLITFGGVIVIGYFDY, the amino acid sequence of LCDR1 is QASQDISNYLN, the amino acid sequence of LCDR2 is DASNLET, and the amino acid sequence of LCDR3 is QQYDNLPIT; or
b) the amino acid sequence of HCDR1 is SYWMS, the amino acid sequence of HCDR2 is NIKQDGSQKYYVDSVKG, the amino acid sequence of HCDR3 is RGNFFFDN, the amino acid sequence of LCDR1 is RASQYVSSYLA, the amino acid sequence of LCDR2 is DASNRAT, and the amino acid sequence of LCDR3 is QQRNNWPPT; or
c) the amino acid sequence of HCDR1 is NHYWS, the amino acid sequence of HCDR2 is YIYYSGSTNYNPSLKS, the amino acid sequence of HCDR3 is GFAY, the amino acid sequence of LCDR1 is RASQGITNYLA, the amino acid sequence of LCDR2 is AASTLQS, and the amino acid sequence of LCDR3 is QKYNSAPWT; or
d) the amino acid sequence of HCDR1 is SYAMN, the amino acid sequence of HCDR2 is AISGSGDSTYSTDSVKG, the amino acid sequence of HCDR3 is EYYDILTGYWDWYFDL, the amino acid sequence of LCDR1 is RASQSINSNLA, the amino acid sequence of LCDR2 is GASTRAT, and the amino acid sequence of LCDR3 is QQYNNWPLT; or
e) the amino acid sequence of HCDR1 is SYGMH, the amino acid sequence of HCDR2 is VMWYDGSDRYSADSVKG, the amino acid sequence of HCDR3 is GYDILTGPDHFDY, the amino acid sequence of LCDR1 is RASQSISSYLN, the amino acid sequence of LCDR2 is AASSLQS, and the amino acid sequence of LCDR3 is QQSYSTPLT; or
f) the amino acid sequence of HCDR1 is SYDMN, the amino acid sequence of HCDR2 is TISGSGSHTYYADSVRG, the amino acid sequence of HCDR3 is EGGATAFDI, the amino acid sequence of LCDR1 is RASQGISSYLA, the amino acid sequence of LCDR2 is VASTLQS, and the amino acid sequence of LCDR3 is QQLNNYPT; or
g) the amino acid sequence of HCDR1 is TYGMH, the amino acid sequence of HCDR2 is LIWYDGINKYYADSVKG, the amino acid sequence of HCDR3 is PYYDILTGYFDY, the amino acid sequence of LCDR1 is RASQSDSSSYLA, the amino acid sequence of LCDR2 is GTSSRAT, and the amino acid sequence of LCDR3 is QQYGSSPLT; or
h) the amino acid sequence of HCDR1 is SYGMH, the amino acid sequence of HCDR2 is VIWYDGTNKYYADSVKG, the amino acid sequence of HCDR3 is DPSLWFGEFPHYYGMDV, the amino acid sequence of LCDR1 is QASQDISNYLN, the amino acid sequence of LCDR2 is DASNLET, and the amino acid sequence of LCDR3 is QQYDNLPLT; or
i) the amino acid sequence of HCDR1 is GYYWS, the amino acid sequence of HCDR2 is EIQHSGSTNYKPSLKS, the amino acid sequence of HCDR3 is LTGDSLLFEY, the amino acid sequence of LCDR1 is RASQSVSSYLA, the amino acid sequence of LCDR2 is DTSNRAT, and the amino acid sequence of LCDR3 is QQRSNWPIT; or
j) the amino acid sequence of HCDR1 is SYGMH, the amino acid sequence of HCDR2 is VIWYDGSNKYYADSVKG, the amino acid sequence of HCDR3 is DSASDYFDY, the amino acid sequence of LCDR1 is RASQSVSSNLA, the amino acid sequence of LCDR2 is GASTRAT, and the amino acid sequence of LCDR3 is QQYSDWPT; or
k) the amino acid sequence of HCDR1 is GYYWS, the amino acid sequence of HCDR2 is EINHSGSTNYKPSLKS, the amino acid sequence of HCDR3 is LTGDSLLFEY, the amino acid sequence of LCDR1 is RASQSVSSYLA, the amino acid sequence of LCDR2 is DTSNRAT, and the amino acid sequence of LCDR3 is QQRSNWPIT.

2. The antibody or antigen-binding fragment or variant thereof of claim 1, comprising a light chain variable region (LCVR) and a heavy chain variable region (HCVR), wherein:
a) the amino acid sequence of the LCVR is
DIQMTQSPSSLSASVGDRVTITCQASQDISNYLNWYQQKPGKAPKLLIYDASNLE
TGVPSRFSGSGSGTDFTFTISSLQPEDIATYYCQQYDNLPITFGQGTRLEIK,
and the amino acid sequence of the HCVR is
EVQLLESGGGLVQPGGSLRLSCAASGFTFSSYAMSWVRQAPGKGLEWVSAISDS
GGSTYYADSVKGRFTISRDNSKNTLYLQMNSLRAEDTAVYYCAQEGLITFGGVI
VIGYFDYWGQGTLVTVSS;
or
b) the amino acid sequence of the LCVR is
EIVLTQSPATLSLSPGERATLSCRASQYVSSYLAWYHQKPGQAPRLLIYDASNRA
TGIPARFSGSGSGTDFTLTISSLEPEDFAVYYCQQRNNWPPTFGQGTKVEIK,
and the amino acid sequence of the HCVR is
EVQLVESGGGLVQPGGSLRLSCAASGFTFSSYWMSWVRQAPGKGLEWVANIKQ
DGSQKYYVDSVKGRFTISRDNAKNSLYLQMNSLRAEDTAVYYCTRRGNFFFDN
WGQGTLVTVSS;
or
c) the amino acid sequence of the LCVR is
DIQMTQSPSSLSASVGDRVTITCRASQGITNYLAWYQQKPGKVPKLLIYAASTLQ
SGVPSRFSGSGSGTDFTLTINSLQPEDVATYYCQKYNSAPWTFGQGTKVEIK,
and the amino acid sequence of the HCVR is
QVQLQESGPGLVKPSETLSLTCTVSGGSISNHYWSWIRQPPGKGLEWIGYIYYSG
STNYNPSLKSRVTISVDTSKNQFSLKLSSVTAADTAVYYCARGFAYWGQGTLVT
VSS;
or
d) the amino acid sequence of the LCVR is
EIVMTLSPATLSVSPGERATLSCRASQSINSNLAWYQQKPGQAPRLLIYGASTRAT
GIPARFSGSGSGTEFTLTISSLQSEDFAVYYCQQYNNWPLTFGGGTKVEIK,
and the amino acid sequence of the HCVR is
EVQLLESGGGLGQPGGSLRLSCAASGFTFSSYAMNWVRQAPGKGLEWVSAISGS
GDSTYSTDSVKGRFTISRDNSKNTLYLQMNSLRAEDTAVYYCAKEYYDILTGYW
DWYFDLWGQGTLVTVSS;
or
e) the amino acid sequence of the LCVR is
DIQMTQSPSSLSASVGDRVTITCRASQSISSYLNWYQQKPGKAPKLLIYAASSLQS
GVPSRFSGSGSGTDFTLTISSLQPEDFATYYCQQSYSTPLTFGGGTKVEIK,
and the amino acid sequence of the HCVR is
QVQLVESGGGVVQPGRSLRLSCAASGFTFSSYGMHWVRQAPGKGLEWVAVMW
YDGSDRYSADSVKGRFTISRDNSKNTLYLQMNSLRAEDTAVYYCARGYDILTGP
DHFDYWGQGTLVTVSS;
or
f) the amino acid sequence of the LCVR is
DIQLTQSPSFLSASVGDRVTITCRASQGISSYLAWYQQKPGKAPKLLIYVASTLQS
GVPSRFSGSGSGTEFTLTISSLQPEDFATYYCQQLNNYPTFGGGTKVEIK,
and the amino acid sequence of the HCVR is
EVQLLESGGGLVQPGGSLRLSCAASGFTFSSYDMNWVRQAPGKGPVWVSTISGS
GSHTYYADSVRGRFTISRDNSKNTLYLQMNSLRAEDTAVYYCAKEGGATAFDI
WGQGTMVTVSS;
or
g) the amino acid sequence of the LCVR is
EIVLTQSPGTLSLSPGERATLSCRASQSDSSSYLAWYQQKPGQAPRLLIYGTSSRA
TGISDRFSGSGSGTDFTLTISRLEPEDFAVYYCQQYGSSPLTFGGGTKVEIK,
and the amino acid sequence of the HCVR is
QVQLVESGGGVVQPGRSLRLSCAASGFTFSTYGMHWVRQAPGKGLEWVALIW
YDGINKYYADSVKGRFTISRDNSKNTLYLQMNSLRAEDTAVYFCARPYYDILTG
YFDYWGQGTLVTVSS;
or
h) the amino acid sequence of the LCVR is
DIQMTQSPSSLSASVGDRVTITCQASQDISNYLNWYQQKPGKAPKLLIYDASNLE
TGVPSRFSGSGSGTDFTFTISSLQPEDIATYYCQQYDNLPLTFGGGTKVEIK,
and the amino acid sequence of the HCVR is
QVQLVESGGGVVQPGRSLRLSCAASGFTFSSYGMHWVRQAPGKGLEWVAVIW
YDGTNKYYADSVKGRFTISRDNSKNTLYLQVNSLRAEDTAVYYCARDPSLWFG
EFPHYYGMDVWGQGTTVTVSS;
or
i) the amino acid sequence of the LCVR is
EIVLTQSPATLSLSPGERATLSCRASQSVSSYLAWYQQKPGQAPRLLIYDTSNRAT
GIPARFSGSGSGTDFTLTVSSLEPEDFAVYYCQQRSNWPITFGQGTRLEIK,
and the amino acid sequence of the HCVR is
QVQLQQWGAGLLKPSETLSLTCAVYGGSFSGYYWSWIRQPPGKGLEWIGEIQHS
GSTNYKPSLKSRVTISVDTSKNQFSLKLSSVTAADTAVYYCAELTGDSLLFEYWG
QGTLVTVSS;
or
j) the amino acid sequence of the LCVR is
EIVMTQSPATLSVSPGERATLSCRASQSVSSNLAWYQQKPGQAPRLLIYGASTRA
TGIPARFSGSGSGTEFTLTISSLQSEDFA VYYCQQYSDWPTFGGGTKVEIR,
and the amino acid sequence of the HCVR is
QVQLVESGGGVVQPGRSLRLSCAASGFTFSSYGMHWVRQAPGKGLEWVAVIW
YDGSNKYYADSVKGRFTISRDTSKNTLYLQMNSLRAEDTAVYYCARDSASDYF
DYWGQGTLVTVSS.
k) the amino acid sequence of the LCVR is
EIVLTQSPATLSLSPGERATLSCRASQSVSSYLAWYQQKPGQAPRLLIYDTSNRAT
GIPARFSGSGSGTDFTLTVSSLEPEDFAVYYCQQRSNWPITFGQGTRLEIK,
and the amino acid sequence of the HCVR is
QVQLQQWGAGLLKPSETLSLTCAVYGGSFSGYYWSWIRQPPGKGLEWIGEINHS
GSTNYKPSLKSRVTISVDTSKNQFSLKLSSVTAADTAVYYCAELTGDSLLFEYWG
QGTLVTVSS.

3. The antibody or antigen-binding fragment thereof of claim 1, comprising a light chain variable region (LCVR) and a heavy chain variable region (HCVR), wherein:
a) the VL region comprises an amino acid sequence that has at least 95% sequence identity to the amino acid sequence
DIQMTQSPSSLSASVGDRVTITCQASQDISNYLNWYQQKPGKAPKLLIYDA
SNLETGVPSRFSGSGSGTDFTFTISSLQPEDIATYYCQQYDNLPITFGQGT
RLEIK,
and the VH region comprises an amino acid sequence that has at least 95% sequence identity to the amino acid sequence
EVQLLESGGGLVQPGGSLRLSCAASGFTFSSYAMSWVRQAPGKGLEWV
SAISDSGGSTYYADSVKGRFTISRDNSKNTLYLQMNSLRAEDTAVYYC
AQEGLITFGGVIVIGYFDYWGQGTLVTVSS;
or
b) the VL region comprises an amino acid sequence that has at least 95% sequence identity to the amino acid sequence
EIVLTQSPATLSLSPGERATLSCRASQYVSSYLAWYHQKPGQAPRLLIYDA
SNRATGIPARFSGSGSGTDFTLTISSLEPEDFAVYYCQQRNNWPPTFGQGT
KVEIK,
and the VH region comprises an amino acid sequence that has at least 95% sequence identity to the amino acid sequence
EVQLVESGGGLVQPGGSLRLSCAASGFTFSSYWMSWVRQAPGKGLEWV
ANIKQDGSQKYYVDSVKGRFTISRDNAKNSLYLQMNSLRAEDTAVYYC
TRRGNFFFDNWGQGTLVTVSS;
or
c) the VL region comprises an amino acid sequence that has at least 95% sequence identity to the amino acid sequence
DIQMTQSPSSLSASVGDRVTITCRASQGITNYLAWYQQKPGKVPKLLIYAA
STLQSGVPSRFSGSGSGTDFTLTINSLQPEDVATYYCQKYNSAPWTFGQGT
KVEIK,
and the VH region comprises an amino acid sequence that has at least 95% sequence identity to the amino acid sequence
QVQLQESGPGLVKPSETLSLTCTVSGGSISNHYWSWIRQPPGKGLEWIGYI
YYSGSTNYNPSLKSRVTISVDTSKNQFSLKLSSVTAADTAVYYCARGFAYW
GQGTLVTVSS;
or
d) the VL region comprises an amino acid sequence that has at least 95% sequence identity to the amino acid sequence
EIVMTLSPATLSVSPGERATLSCRASQSINSNLAWYQQKPGQAPRLLIYGA
STRATGIPARFSGSGSGTEFTLTISSLQSEDFAVYYCQQYNNWPLTFGGGT
KVEIK,
and the VH region comprises an amino acid sequence that has at least 95% sequence identity to the amino acid sequence
EVQLLESGGGLGQPGGSLRLSCAASGFTFSSYAMNWVRQAPGKGLEWVSAI
SGSGDSTYSTDSVKGRFTISRDNSKNTLYLQMNSLRAEDTAVYYCAKEYYD
ILTGYWDWYFDLWGQGTLVTVSS;
or
e) the VL region comprises an amino acid sequence that has at least 95% sequence identity to the amino acid sequence
DIQMTQSPSSLSASVGDRVTITCRASQSISSYLNWYQQKPGKAPKLLIYAA
SSLQSGVPSRFSGSGSGTDFTLTISSLQPEDFATYYCQQSYSTPLTFGGGT
KVEIK,
and the VH region comprises an amino acid sequence that has at least 95% sequence identity to the amino acid sequence
QVQLVESGGGVVQPGRSLRLSCAASGFTFSSYGMHWVRQAPGKGLEWVAVM
WYDGSDRYSADSVKGRFTISRDNSKNTLYLQMNSLRAEDTAVYYCARGYDI
LTGPDHFDYWGQGTLVTVSS;
or
f) the VL region comprises an amino acid sequence that has at least 95% sequence identity to the amino acid sequence
DIQLTQSPSFLSASVGDRVTITCRASQGISSYLAWYQQKPGKAPKLLIYVA
STLQSGVPSRFSGSGSGTEFTLTISSLQPEDFATYYCQQLNNYPTFGGGTK
VEIK,
and the VH region comprises an amino acid sequence that has at least 95% sequence identity to the amino acid sequence
EVQLLESGGGLVQPGGSLRLSCAASGFTFSSYDMNWVRQAPGKGPVWVSTI
SGSGSHTYYADSVRGRFTISRDNSKNTLYLQMNSLRAEDTAVYYCAKEGGA
TAFDIWGQGTMVTVSS;
or
g) the VL region comprises an amino acid sequence that has at least 95% sequence identity to the amino acid sequence
EIVLTQSPGTLSLSPGERATLSCRASQSDSSSYLAWYQQKPGQAPRLL
IYGTSSRATGISDRFSGSGSGTDFTLTISRLEPEDFAVYYCQQYGSSP
LTFGGGTKVEIK,
and the VH region comprises an amino acid sequence that has at least 95% sequence identity to the amino acid sequence
QVQLVESGGGVVQPGRSLRLSCAASGFTFSTYGMHWVRQAPGKGLEWV
ALIWYDGINKYYADSVKGRFTISRDNSKNTLYLQMNSLRAEDTAVYF
CARPYYDILTGYFDYWGQGTLVTVSS;
or
h) the VL region comprises an amino acid sequence that has at least 95% sequence identity to the amino acid sequence
DIQMTQSPSSLSASVGDRVTITCQASQDISNYLNWYQQKPGKAPKLLI
YDASNLETGVPSRFSGSGSGTDFTFTISSLQPEDIATYYCQQYDNLPL
TFGGGTKVEIK,
and the VH region comprises an amino acid sequence that has at least 95% sequence identity to the amino acid sequence
QVQLVESGGGVVQPGRSLRLSCAASGFTFSSYGMHWVRQAPGKGLEWV
AVIWYDGTNKYYADSVKGRFTISRDNSKNTLYLQVNSLRAEDTAVYYC
ARDPSLWFGEFPHYYGMDVWGQGTTVTVSS;
or
i) the VL region comprises an amino acid sequence that has at least 95% sequence identity to the amino acid sequence
EIVLTQSPATLSLSPGERATLSCRASQSVSSYLAWYQQKPGQAPRLLI
YDTSNRATGIPARFSGSGSGTDFTLTVSSLEPEDFAVYYCQQRSNWPI
TFGQGTRLEIK,
and the VH region comprises an amino acid sequence that has at least 95% sequence identity to the amino acid sequence
QVQLQQWGAGLLKPSETLSLTCAVYGGSFSGYYWSWIRQPPGKGLEWI
GEIQHSGSTNYKPSLKSRVTISVDTSKNQFSLKLSSVTAADTAVYYCA
ELTGDSLLFEYWGQGTLVTVSS;
or
j) the VL region comprises an amino acid sequence that has at least 95% sequence identity to the amino acid sequence
EIVMTQSPATLSVSPGERATLSCRASQSVSSNLAWYQQKPGQAPRLLI
YGASTRATGIPARFSGSGSGTEFTLTISSLQSEDFAVYYCQQYSDWPT
FGGGTKVEIR,
and the VH region comprises an amino acid sequence that has at least 95% sequence identity to the amino acid sequence
QVQLVESGGGVVQPGRSLRLSCAASGFTFSSYGMHWVRQAPGKGLEWV
AVIWYDGSNKYYADSVKGRFTISRDTSKNTLYLQMNSLRAEDTAVYYC
ARDSASDYFDYWGQGTLVTVSS.
k) the VL region comprises an amino acid sequence that has at least 95% sequence identity to the amino acid sequence
EIVLTQSPATLSLSPGERATLSCRASQSVSSYLAWYQQKPGQAPRLLI
YDTSNRATGIPARFSGSGSGTDFTLTVSSLEPEDFAVYYCQQRSNWPI
TFGQGTRLEIK,
and the VH region comprises an amino acid sequence that has at least 95% sequence identity to the amino acid sequence
QVQLQQWGAGLLKPSETLSLTCAVYGGSFSGYYWSWIRQPPGKGLEWI
GEINHSGSTNYKPSLKSRVTISVDTSKNQFSLKLSSVTAADTAVYYCA
ELTGDSLLFEYWGQGTLVTVSS.

4. The antibody or antigen-binding fragment or variant thereof of claim 1, wherein the fragment comprises an F(ab), an Fv, or an scFv.

5. The antibody or antigen-binding fragment or variant thereof of claim 1, wherein the fragment comprises a VhH.

6. The antibody or antigen-binding fragment or variant thereof of claim 1, wherein the constant domain is IgG.

7. The antibody or antigen-binding fragment or variant thereof of claim 6, wherein the constant region is IgG4.

8. (canceled)

9. A pharmaceutical composition, comprising: the antibody of claim 1, and one or more pharmaceutically acceptable carriers, diluents, or excipients.

10. A method of treating cancer in a subject, the method comprising administering to a subject in need thereof an effective amount of the antibody of claim 1.

11. The method of claim 10, further comprising administering to the subject an angiogenesis inhibitor, a checkpoint blockade inhibitor, or a combination thereof.

12. The method of claim 11, wherein the angiogenesis inhibitor comprises bevacizumab.

13. The method of claim 11, wherein the checkpoint blockade inhibitor comprises ipilimumab.

14-15. (canceled)

16. A method of decreasing immune suppressive effects of cancer-associated fibroblasts in a subject, comprising administering to the subject an effective amount of the antibody of claim 1.

17-18. (canceled)

19. A nucleic acid encoding the antibody of claim 1.

20. The nucleic acid of claim 19, wherein:
a) the nucleic acid sequence encoding HCDR1 is AGCTATGCCATGAGC or a degenerate variant thereof; the nucleic acid sequence encoding HCDR2 is GCTATTAGTGATAGTGGTGGTAGCACATACTACGCAGACTCCGTGAAGGGC, or a degenerate variant thereof, the nucleic acid sequence encoding HCDR3 is GAGGGTTTGATTACGTTTGGGGGAGTTATCGTTATAGGCTACTTTGACTAC, or a degenerate variant thereof,
the nucleic acid sequence encoding LCDR1 is CAGGCGAGTCAGGACATTAGCAACTATTTAAAT, or a degenerate variant thereof, the nucleic acid sequence encoding LCDR2 is GATGCATCCAATTTGGAAACA, or a degenerate variant thereof, and the nucleic acid sequence encoding LCDR3 is CAACAGTATGATAATCTCCCGATCACC, or a degenerate variant thereof, or
b) the nucleic acid sequence encoding HCDR1 is AGCTATTGGATGAGC, or a degenerate variant thereof, the nucleic acid sequence encoding HCDR2 is AATATAAAGCAAGATGGAAGTCAGAAATACTATGTGGACTCTGTGAAGGGC, or a degenerate variant thereof, the nucleic acid sequence encoding HCDR3 is CGTGGGAACTTCTTCTTTGACAAT, or a degenerate variant thereof,
the nucleic acid sequence encoding LCDR1 is AGGGCCAGTCAGTATGTTAGCAGCTACTTAGCC, or a degenerate variant thereof, the nucleic acid sequence encoding LCDR2 is GATGCATCCAACAGGGCCACT, or a degenerate variant thereof, and the nucleic acid sequence encoding LCDR3 is CAGCAGCGTAACAACTGGCCTCCGACG, or a degenerate variant thereof, or
c) the nucleic acid sequence encoding HCDR1 is AATCACTACTGGAGC, or a degenerate variant thereof, the nucleic acid sequence encoding HCDR2 is TATATCTATTACAGTGGGAGCACCAACTACAACCCCTCCCTCAAGAGT, or a degenerate variant thereof, the nucleic acid sequence encoding HCDR3 is GGGTTTGCTTAC, or a degenerate variant thereof,
the nucleic acid sequence encoding LCDR1 is CGGGCGAGTCAGGGCATTACCAATTATTTAGCC, or a degenerate variant thereof, the nucleic acid sequence encoding LCDR2 is GCTGCATCCACTTTGCAATCA, or a degenerate variant thereof, and the nucleic acid sequence encoding LCDR3 is CAAAAGTATAACAGTGCCCCGTGGACG, or a degenerate variant thereof; or
d) the nucleic acid sequence encoding HCDR1 is AGCTATGCCATGAAC, or a degenerate variant thereof, the nucleic acid sequence encoding HCDR2 is GCTATCAGTGGCAGTGGTGATAGCACATACTCCACAGACTCCGTGAAGGGC, or a degenerate variant thereof, the nucleic acid sequence encoding HCDR3 is GAGTATTACGATATTTTGACTGGTTATTGGGACTGGTACTTCGATCTC, or a degenerate variant thereof,
the nucleic acid sequence encoding LCDR1 is AGGGCCAGTCAGAGTATTAACAGCAACTTAGCC, or a degenerate variant thereof, the nucleic acid sequence encoding LCDR2 is GGTGCATCCACCAGGGCCACT, or a degenerate variant thereof, and the nucleic acid sequence encoding LCDR3 is CAGCAGTATAATAACTGGCCGCTCACT, or a degenerate variant thereof; or
e) the nucleic acid sequence encoding HCDR1 is AGCTATGGCATGCAT, or a degenerate variant thereof, the nucleic acid sequence encoding HCDR2 is GTTATGTGGTATGATGGAAGTGATAGATACTCTGCAGACTCCGTGAAGGGC, or a degenerate variant thereof, the nucleic acid sequence encoding HCDR3 is GGGTACGATATTTTGACTGGTCCCGACCACTTTGACTAC, or a degenerate variant thereof,
the nucleic acid sequence encoding LCDR1 is CGGGCAAGTCAGAGCATTAGCAGTTATTTAAAT, or a degenerate variant thereof, the nucleic acid sequence encoding LCDR2 is GCTGCATCCAGTTTGCAAAGT, or a degenerate variant thereof, and the nucleic acid sequence encoding LCDR3 is CAACAGAGTTACAGTACCCCGCTCACT, or a degenerate variant thereof, or
f) the nucleic acid sequence encoding HCDR1 is AGCTATGACATGAAC, or a degenerate variant thereof, the nucleic acid sequence encoding HCDR2 is ACTATTAGTGGTAGTGGTAGTCACACATACTACGCAGACTCCGTGAGGGGC, or a degenerate variant thereof, the nucleic acid sequence encoding HCDR3 is GAGGGGGGAGCTACTGCTTTTGATATC, or a degenerate variant thereof,
the nucleic acid sequence encoding LCDR1 is CGGGCCAGTCAGGGCATTAGCAGTTATTTAGCC, or a degenerate variant thereof, the nucleic acid sequence encoding LCDR2 is GTTGCATCCACTTTGCAAAGT, or a degenerate variant thereof, and the nucleic acid sequence encoding LCDR3 is CAACAGCTTAATAATTACCCCACT, or a degenerate variant thereof; or
g) the nucleic acid sequence encoding HCDR1 is ACCTATGGCATGCAC, or a degenerate variant thereof, the nucleic acid sequence encoding HCDR2 is CTTATATGGTATGATGGAATTAATAAATACTATGCGGACTCCGTGAAGGGC, or a degenerate variant thereof, the nucleic acid sequence encoding HCDR3 is CCCTATTACGATATTTTGACTGGTTATTTTGACTAC, or a degenerate variant thereof,
the nucleic acid sequence encoding LCDR1 is AGGGCCAGTCAGAGTGATAGCAGCAGCTACTTAGCC, or a degenerate variant thereof, the nucleic acid sequence encoding LCDR2 is GGTACATCCAGTAGGGCCACT, or a degenerate variant thereof, and the nucleic acid sequence encoding LCDR3 is CAGCAGTATGGTAGCTCACCGCTCACT, or a degenerate variant thereof; or
h) the nucleic acid sequence encoding HCDR1 is AGCTATGGCATGCAC, or a degenerate variant thereof, the nucleic acid sequence encoding HCDR2 is GTTATATGGTATGATGGAACTAATAAATACTATGCAGACTCCGTGAAGGGC, or a degenerate variant thereof, the nucleic acid sequence encoding HCDR3 is GATCCCTCCTTATGGTTCGGGGAGTTCCCTCATTACTACGGTATGGACGTC, or a degenerate variant thereof,
the nucleic acid sequence encoding LCDR1 is CAGGCGAGTCAGGACATTAGCAATTATTTAAAT, or a degenerate variant thereof, the nucleic acid sequence encoding LCDR2 is GATGCATCCAATTTGGAAACA, or a degenerate variant thereof, and the nucleic acid sequence encoding LCDR3 is CAACAGTATGATAATCTCCCGCTCACT, or a degenerate variant thereof; or
i) the nucleic acid sequence encoding HCDR1 is GGTTACTACTGGAGC, or a degenerate variant thereof, the nucleic acid sequence encoding HCDR2 is GAAATCCAACATAGTGGAAGCACCAACTACAAACCGTCCCTCAAGAGT, or a degenerate variant thereof, the nucleic acid sequence encoding HCDR3 is CTAACTGGGGATTCCCTTTTGTTTGAGTAC, or a degenerate variant thereof,
the nucleic acid sequence encoding LCDR1 is AGGGCCAGTCAGAGTGTTAGCAGCTACTTAGCC, or a degenerate variant thereof, the nucleic acid sequence encoding LCDR2 is GATACATCCAACAGGGCCACT, or a degenerate variant thereof, and the nucleic acid sequence encoding LCDR3 is CAGCAGCGTAGCAACTGGCCGATCACC, or a degenerate variant thereof; or
j) the nucleic acid sequence encoding HCDR1 is AGCTATGGCATGCAC, or a degenerate variant thereof, the nucleic acid sequence encoding HCDR2 is GTTATATGGTATGATGGAAGTAATAAATACTATGCAGACTCCGTGAAGGGC, or a degenerate variant thereof, the nucleic acid sequence encoding HCDR3 is GATAGCGCCTCCGACTACTTTGACTAC, or a degenerate variant thereof,
the nucleic acid sequence encoding LCDR1 is AGGGCCAGTCAGAGTGTTAGCAGCAACTTAGCC, or a degenerate variant thereof, the nucleic acid sequence encoding LCDR2 GGTGCATCCACCAGGGCCACT, or a degenerate variant thereof, and the nucleic acid sequence encoding LCDR3 is CAGCAGTATAGTGACTGGCCCACT, or a degenerate variant thereof; or
k) the nucleic acid sequence encoding HCDR1 is GGTTACTACTGGAGC, or a degenerate variant thereof, the nucleic acid sequence encoding HCDR2 is GAAATCAATCATAGTGGAAGCACCAACTACAAACCGTCCCTCAAGAGT, or a degenerate variant thereof, the nucleic acid sequence encoding HCDR3 is CTAACTGGGGATTCCCTTTTGTTTGAGTAC, or a degenerate variant thereof,
the nucleic acid sequence encoding LCDR1 is AGGGCCAGTCAGAGTGTTAGCAGCTACTTAGCC, or a degenerate variant thereof, the nucleic acid sequence encoding LCDR2 is GATACATCCAACAGGGCCACT, or a degenerate variant thereof, and the nucleic acid sequence encoding LCDR3 is CAGCAGCGTAGCAACTGGCCGATCACC, or a degenerate variant thereof; or

21. The nucleic acid of claim 19, wherein:
a) the nucleic acid sequence encoding LCVR is GACATCCAGATGACCCAGTCTCCATCCTCCCTGTCTGCATCTGTAGGAGACAG AGTCACCATCACTTGCCAGGCGAGTCAGGACATTAGCAACTATTTAAATTGG TATCAGCAGAAACCAGGGAAAGCCCCTAAGCTCCTGATCTACGATGCATCCA ATTTGGAAACAGGGGTCCCATCAAGGTTCAGTGGAAGTGGATCTGGGACAGA TTTTACTTTCACCATCAGCAGCCTGCAGCCTGAAGATATTGCAACATATTACT GTCAACAGTATGATAATCTCCCGATCACCTTCGGCCAAGGGACACGACTGGA GATTAAA, or a degenerate variant thereof, and
the nucleic acid sequence encoding HCVR is GAGGTGCAGCTGTTGGAGTCTGGGGGAGGCTTGGTACAGCCTGGGGGGTCCC TGAGACTCTCCTGTGCAGCCTCTGGATTCACCTTTAGCAGCTATGCCATGAGC TGGGTCCGCCAGGCTCCAGGGAAGGGGCTGGAGTGGGTCTCAGCTATTAGTG ATAGTGGTGGTAGCACATACTACGCAGACTCCGTGAAGGGCCGGTTCACCAT CTCCAGAGACAATTCCAAGAACACGCTGTATCTGCAAATGAACAGCCTGAGA GCCGAGGACACGGCCGTATATTACTGTGCGCAGGAGGGTTTGATTACGTTTG GGGGAGTTATCGTTATAGGCTACTTTGACTACTGGGGCCAGGGAACCCTGGT CACCGTCTCCTCA, or a degenerate variant thereof, or
b) the nucleic acid sequence encoding LCVR is GAAATTGTGTTGACACAGTCTCCAGCCACCCTGTCTTTGTCTCCAGGGGAAAG AGCCACCCTCTCCTGCAGGGCCAGTCAGTATGTTAGCAGCTACTTAGCCTGGT ACCACCAGAAACCTGGCCAGGCTCCCAGGCTCCTCATCTACGATGCATCCAA CAGGGCCACTGGCATCCCAGCCAGGTTCAGTGGCAGTGGGTCTGGGACAGAC TTCACTCTCACCATCAGCAGCCTAGAGCCTGAAGATTTTGCAGTTTATTACTG TCAGCAGCGTAACAACTGGCCTCCGACGTTCGGCCAAGGGACCAAGGTGGAA ATCAAA, or a degenerate variant thereof, and
the nucleic acid sequence encoding HCVR is GAGGTGCAGCTGGTGGAGTCTGGGGGAGGCTTGGTCCAGCCTGGGGGGTCCC TGAGACTCTCCTGTGCAGCCTCTGGATTCACCTTTAGTAGCTATTGGATGAGC TGGGTCCGCCAGGCTCCAGGGAAGGGGCTGGAATGGGTGGCCAATATAAAG CAAGATGGAAGTCAGAAATACTATGTGGACTCTGTGAAGGGCCGATTCACCA TCTCCAGAGACAACGCCAAGAACTCACTGTATCTGCAAATGAACAGCCTGAG AGCCGAGGACACGGCTGTGTATTACTGTACGAGACGTGGGAACTTCTTCTTTG ACAATTGGGGCCAGGGAACCCTGGTCACCGTCTCCTCA, or a degenerate variant thereof; or
c) the nucleic acid sequence encoding LCVR is GACATCCAGATGACCCAGTCTCCATCCTCCCTGTCTGCATCTGTAGGAGACAG AGTCACCATCACTTGCCGGGCGAGTCAGGGCATTACCAATTATTTAGCCTGGT ATCAGCAGAAACCAGGGAAAGTTCCTAAGCTCCTGATCTATGCTGCATCCAC TTTGCAATCAGGGGTCCCATCTCGGTTCAGTGGCAGTGGATCTGGGACAGATT TCACTCTCACCATCAACAGCCTGCAGCCTGAAGATGTTGCAACTTATTACTGT CAAAAGTATAACAGTGCCCCGTGGACGTTCGGCCAAGGGACCAAGGTGGAA ATCAAA, or a degenerate variant thereof, and
the nucleic acid sequence encoding HCVR is CAGGTGCAGCTGCAGGAGTCGGGCCCAGGACTGGTGAAGCCTTCGGAGACCC TGTCCCTCACTTGCACTGTCTCTGGTGGCTCCATCAGTAATCACTACTGGAGC TGGATTCGGCAGCCCCCAGGGAAGGGACTGGAGTGGATTGGGTATATCTATT ACAGTGGGAGCACCAACTACAACCCCTCCCTCAAGAGTCGAGTCACCATATC AGTAGACACGTCCAAGAACCAGTTCTCCCTGAAGCTGAGCTCTGTGACCGCT GCGGACACGGCCGTGTATTACTGTGCGAGGGGGTTTGCTTACTGGGGCCAAG GCACTCTGGTCACTGTCTCTTCA, or a degenerate variant thereof; or
d) the nucleic acid sequence encoding LCVR is GAAATAGTGATGACGCTGTCTCCAGCCACCCTGTCTGTGTCTCCAGGGGAAA GAGCCACCCTCTCCTGCAGGGCCAGTCAGAGTATTAACAGCAACTTAGCCTG GTACCAGCAGAAACCTGGCCAGGCTCCCAGGCTCCTCATCTATGGTGCATCC ACCAGGGCCACTGGTATCCCAGCCAGGTTCAGTGGCAGTGGGTCTGGGACAG AGTTCACTCTCACCATCAGCAGCCTGCAGTCTGAAGATTTTGCAGTTTATTAC TGTCAGCAGTATAATAACTGGCCGCTCACTTTCGGCGGAGGGACCAAGGTGG AGATCAAA, or a degenerate variant thereof, and
the nucleic acid sequence encoding HCVR is GAGGTGCAGCTGTTGGAATCTGGGGGAGGCTTGGGACAGCCTGGGGGGTCCC TGAGACTCTCCTGTGCAGCCTCTGGATTCACCTTTAGCAGCTATGCCATGAAC TGGGTCCGCCAGGCTCCAGGGAAGGGGCTGGAGTGGGTCTCAGCTATCAGTG GCAGTGGTGATAGCACATACTCCACAGACTCCGTGAAGGGCCGGTTCACCAT CTCCAGAGACAATTCCAAGAACACGCTGTATCTGCAAATGAACAGCCTGAGA GCCGAGGACACGGCCGTTTATTACTGTGCGAAAGAGTATTACGATATTTTGA CTGGTTATTGGGACTGGTACTTCGATCTCTGGGGCCAAGGCACCCTGGTCACT GTCTCCTCA, or a degenerate variant thereof; or
e) the nucleic acid sequence encoding LCVR is GACATCCAGATGACCCAGTCTCCATCCTCCCTGTCTGCATCTGTAGGAGACAG AGTCACCATCACTTGCCGGGCAAGTCAGAGCATTAGCAGTTATTTAAATTGGT ATCAGCAGAAACCAGGGAAAGCCCCTAAGCTCCTGATCTATGCTGCATCCAG TTTGCAAAGTGGGGTCCCATCGAGGTTCAGTGGCAGTGGATCTGGGACAGAT TTCACTCTCACCATCAGCAGTCTGCAACCTGAAGATTTTGCAACTTACTATTG TCAACAGAGTTACAGTACCCCGCTCACTTTCGGCGGAGGGACCAAGGTGGAG ATCAAA, or a degenerate variant thereof, and
the nucleic acid sequence encoding HCVR is CAGGTGCAGCTGGTGGAGTCTGGGGGAGGCGTGGTCCAGCCTGGGAGGTCCC TGAGACTCTCCTGTGCAGCGTCTGGATTCACCTTCAGTAGCTATGGCATGCAT TGGGTCCGCCAGGCTCCAGGCAAGGGGCTGGAGTGGGTGGCAGTTATGTGGT ATGATGGAAGTGATAGATACTCTGCAGACTCCGTGAAGGGCCGATTCACCAT CTCCAGAGACAATTCCAAGAACACGCTGTATTTGCAAATGAACAGCCTGAGA GCCGAGGACACGGCTGTGTATTACTGTGCGAGGGGGTACGATATTTTGACTG GTCCCGACCACTTTGACTACTGGGGCCAGGGAACCCTGGTCACCGTCTCCTCA, or a degenerate variant thereof; or
f) the nucleic acid sequence encoding LCVR is GACATCCAGTTGACCCAGTCTCCATCCTTCCTGTCTGCATCTGTAGGAGACAG AGTCACCATCACTTGCCGGGCCAGTCAGGGCATTAGCAGTTATTTAGCCTGGT ATCAGCAAAAACCAGGGAAAGCCCCTAAGCTCCTGATCTATGTTGCATCCAC TTTGCAAAGTGGGGTCCCATCAAGGTTCAGCGGCAGTGGATCTGGGACAGAA TTCACTCTCACAATCAGCAGCCTGCAGCCTGAAGATTTTGCAACTTATTACTG TCAACAGCTTAATAATTACCCCACTTTCGGCGGAGGGACCAAGGTGGAGATC AAA, or a degenerate variant thereof, and
the nucleic acid sequence encoding HCVR is GAAGTACAGTTGTTGGAGTCTGGGGGAGGCTTGGTACAGCCTGGGGGGTCCC TGAGACTCTCCTGTGCAGCCTCTGGATTCACCTTTAGCAGCTATGACATGAAC TGGGTCCGCCAGGCTCCAGGGAAGGGGCCGGTGTGGGTCTCAACTATTAGTG GTAGTGGTAGTCACACATACTACGCAGACTCCGTGAGGGGCCGGTTCACCAT CTCCAGAGACAACTCCAAGAACACGCTGTATCTGCAAATGAACAGCCTGAGA GCCGAGGACACGGCCGTATATTACTGTGCGAAAGAGGGGGGAGCTACTGCTT TTGATATCTGGGGCCAAGGGACAATGGTCACCGTCTCTTCA, or a degenerate variant thereof; or
g) the nucleic acid sequence encoding LCVR is GAAATTGTGTTGACGCAGTCTCCAGGCACCCTGTCTTTGTCTCCAGGGGAAAG AGCCACCCTCTCCTGCAGGGCCAGTCAGAGTGATAGCAGCAGCTACTTAGCC TGGTATCAGCAGAAACCTGGCCAGGCTCCCAGGCTCCTCATATATGGTACAT CCAGTAGGGCCACTGGCATCTCAGACAGGTTCAGTGGCAGTGGGTCTGGGAC AGACTTCACTCTCACCATCAGCAGACTGGAGCCTGAAGATTTTGCAGTGTATT ACTGTCAGCAGTATGGTAGCTCACCGCTCACTTTCGGCGGAGGGACCAAGGT GGAGATCAAA, or a degenerate variant thereof, and
the nucleic acid sequence encoding HCVR is CAGGTGCAGCTGGTGGAGTCTGGGGGAGGCGTGGTCCAGCCTGGGAGGTCCC TGAGACTCTCCTGTGCAGCGTCTGGATTCACCTTCAGTACCTATGGCATGCAC TGGGTCCGCCAGGCTCCAGGCAAGGGGCTGGAGTGGGTGGCACTTATATGGT ATGATGGAATTAATAAATACTATGCGGACTCCGTGAAGGGCCGATTCACCAT CTCCAGAGACAATTCCAAGAACACGCTGTATCTGCAAATGAACAGCCTGAGA GCCGAGGACACGGCTGTGTATTTCTGTGCGAGACCCTATTACGATATTTTGAC TGGTTATTTTGACTACTGGGGCCAGGGAACCCTGGTCACCGTCTCCTCA, or a degenerate variant thereof; or
h) the nucleic acid sequence encoding LCVR is GACATCCAGATGACCCAGTCTCCATCCTCCCTGTCTGCATCTGTAGGAGACAG AGTCACCATCACTTGCCAGGCGAGTCAGGACATTAGCAATTATTTAAATTGGT ATCAGCAGAAACCAGGGAAAGCCCCTAAGCTCCTGATCTACGATGCATCCAA TTTGGAAACAGGGGTCCCATCAAGGTTCAGTGGAAGTGGATCTGGGACAGAT TTTACTTTCACCATCAGCAGCCTGCAGCCTGAAGATATTGCAACATATTACTG TCAACAGTATGATAATCTCCCGCTCACTTTCGGCGGAGGGACCAAGGTGGAG ATCAAA, or a degenerate variant thereof, and
the nucleic acid sequence encoding HCVR is CAGGTGCAGCTGGTGGAGTCTGGGGGAGGCGTGGTCCAGCCTGGGAGGTCCC TGAGACTCTCCTGTGCAGCGTCTGGATTCACCTTCAGTAGCTATGGCATGCAC TGGGTCCGCCAGGCTCCAGGCAAGGGGCTGGAGTGGGTGGCAGTTATATGGT ATGATGGAACTAATAAATACTATGCAGACTCCGTGAAGGGCCGATTCACCAT CTCCAGAGACAATTCCAAGAACACGCTGTATCTGCAAGTGAACAGCCTGAGA GCCGAGGACACGGCTGTGTATTACTGTGCGAGAGATCCCTCCTTATGGTTCGG GGAGTTCCCTCATTACTACGGTATGGACGTCTGGGGCCAAGGGACCACGGTC ACCGTCTCCTCA, or a degenerate variant thereof, or
i) the nucleic acid sequence encoding LCVR is GAAATTGTGTTGACACAGTCTCCAGCCACCCTGTCTTTGTCTCCAGGGGAAAG AGCCACCCTCTCCTGCAGGGCCAGTCAGAGTGTTAGCAGCTACTTAGCCTGG TACCAACAGAAACCTGGCCAGGCTCCCAGGCTCCTCATCTATGATACATCCA ACAGGGCCACTGGCATCCCAGCCAGGTTCAGTGGCAGTGGGTCTGGGACAGA CTTCACTCTCACCGTCAGCAGCCTAGAGCCTGAAGATTTTGCAGTTTATTACT GTCAGCAGCGTAGCAACTGGCCGATCACCTTCGGCCAAGGGACACGACTGGA GATTAAA, or a degenerate variant thereof, and
the nucleic acid sequence encoding HCVR is CAGGTGCAGCTACAGCAGTGGGGCGCAGGACTGTTGAAGCCTTCGGAGACCC TGTCCCTCACCTGCGCTGTCTATGGTGGGTCCTTCAGTGGTTACTACTGGAGC TGGATACGCCAGCCCCCAGGGAAGGGGCTGGAGTGGATTGGGGAAATCCAA CATAGTGGAAGCACCAACTACAAACCGTCCCTCAAGAGTCGAGTCACCATAT CAGTAGACACGTCCAAGAACCAGTTCTCCCTGAAGCTGAGCTCTGTGACCGC CGCGGACACGGCTGTGTATTACTGTGCGGAGCTAACTGGGGATTCCCTTTTGT TTGAGTACTGGGGCCAGGGAACCCTGGTCACCGTCTCCTCA, or a degenerate variant thereof; or
j) the nucleic acid sequence encoding LCVR is GAAATAGTGATGACGCAGTCTCCAGCCACCCTGTCTGTGTCTCCAGGGGAAA GAGCCACCCTCTCCTGCAGGGCCAGTCAGAGTGTTAGCAGCAACTTAGCCTG GTACCAGCAGAAACCTGGCCAGGCTCCCAGGCTCCTCATCTATGGTGCATCC ACCAGGGCCACTGGTATCCCAGCCAGATTCAGTGGCAGTGGGTCTGGGACAG AGTTCACTCTCACCATCAGCAGCCTGCAGTCTGAAGATTTTGCAGTTTATTAC TGTCAGCAGTATAGTGACTGGCCCACTTTCGGCGGAGGGACCAAGGTGGAGA TCAGA, or a degenerate variant thereof, and
the nucleic acid sequence encoding HCVR is CAGGTGCAGCTGGTGGAGTCTGGGGGAGGCGTGGTCCAGCCTGGGAGGTCCC TGAGACTCTCCTGTGCAGCGTCTGGATTCACCTTCAGTAGCTATGGCATGCAC TGGGTCCGCCAGGCTCCAGGCAAGGGGCTGGAGTGGGTGGCAGTTATATGGT ATGATGGAAGTAATAAATACTATGCAGACTCCGTGAAGGGCCGATTCACCAT CTCCAGAGACACTTCCAAGAACACACTGTATCTGCAAATGAACAGCCTGAGA GCCGAGGACACGGCTGTGTATTACTGTGCGAGAGATAGCGCCTCCGACTACT TTGACTACTGGGGCCAGGGAACCCTGGTCACCGTCTCCTCA, or a degenerate variant thereof; or
k) the nucleic acid sequence encoding the LCVR is GAAATTGTGTTGACACAGTCTCCAGCCACCCTGTCTTTGTCTCCAGGGGAAAG AGCCACCCTCTCCTGCAGGGCCAGTCAGAGTGTTAGCAGCTACTTAGCCTGG TACCAACAGAAACCTGGCCAGGCTCCCAGGCTCCTCATCTATGATACATCCA ACAGGGCCACTGGCATCCCAGCCAGGTTCAGTGGCAGTGGGTCTGGGACAGA CTTCACTCTCACCGTCAGCAGCCTAGAGCCTGAAGATTTTGCAGTTTATTACT GTCAGCAGCGTAGCAACTGGCCGATCACCTTCGGCCAAGGGACACGACTGGA GATTAAA, or a degenerate variant thereof, and
the nucleic acid sequence encoding HCVR is CAGGTGCAGCTACAGCAGTGGGGCGCAGGACTGTTGAAGCCTTCGGAGACCCTGTC CCTCACCTGCGCTGTCTATGGTGGGTCCTTCAGTGGTTACTACTGGAGCTGGATACG CCAGCCCCCAGGGAAGGGGCTGGAGTGGATTGGGGAAATCAATCATAGTGGAAGCA CCAACTACAAACCGTCCCTCAAGAGTCGAGTCACCATATCAGTAGACACGTCCAAG AACCAGTTCTCCCTGAAGCTGAGCTCTGTGACCGCCGCGGACACGGCTGTGTATTAC TGTGCGGAGCTAACTGGGGATTCCCTTTTGTTTGAGTACTGGGGCCAGGGAACCCTG GTCACCGTCTCCTCA, or a degenerate variant thereof.

22. A vector comprising the nucleic acid of claim 19.

23. A cell comprising the nucleic acid of claim 19.


SEQUENCE LISTING (156 sequences)

SEQ ID NO: 1 - protein, 5 aa, synthetic construct
SYAMS

SEQ ID NO: 2 - protein, 17 aa, synthetic construct
AISDSGGSTYYADSVKG

SEQ ID NO: 3 - protein, 17 aa, synthetic construct
EGLITFGGVIVIGYFDY

SEQ ID NO: 4 - protein, 11 aa, synthetic construct
QASQDISNYLN

SEQ ID NO: 5 - protein, 7 aa, synthetic construct
DASNLET

SEQ ID NO: 6 - protein, 9 aa, synthetic construct
QQYDNLPIT

SEQ ID NO: 7 - protein, 5 aa, synthetic construct
SYWMS

SEQ ID NO: 8 - protein, 17 aa, synthetic construct
NIKQDGSQKYYVDSVKG

SEQ ID NO: 9 - protein, 8 aa, synthetic construct
RGNFFFDN

SEQ ID NO: 10 - protein, 11 aa, synthetic construct
RASQYVSSYLA

SEQ ID NO: 11 - protein, 7 aa, synthetic construct
DASNRAT

SEQ ID NO: 12 - protein, 9 aa, synthetic construct
QQRNNWPPT

SEQ ID NO: 13 - protein, 5 aa, synthetic construct
NHYWS

SEQ ID NO: 14 - protein, 16 aa, synthetic construct
YIYYSGSTNYNPSLKS

SEQ ID NO: 15 - protein, 4 aa, synthetic construct
GFAY

SEQ ID NO: 16 - protein, 11 aa, synthetic construct
RASQGITNYLA

SEQ ID NO: 17 - protein, 7 aa, synthetic construct
AASTLQS

SEQ ID NO: 18 - protein, 9 aa, synthetic construct
QKYNSAPWT

SEQ ID NO: 19 - protein, 5 aa, synthetic construct
SYAMN

SEQ ID NO: 20 - protein, 17 aa, synthetic construct
AISGSGDSTYSTDSVKG

SEQ ID NO: 21 - protein, 16 aa, synthetic construct
EYYDILTGYWDWYFDL

SEQ ID NO: 22 - protein, 11 aa, synthetic construct
RASQSINSNLA

SEQ ID NO: 23 - protein, 7 aa, synthetic construct
GASTRAT

SEQ ID NO: 24 - protein, 9 aa, synthetic construct
QQYNNWPLT

SEQ ID NO: 25 - protein, 5 aa, synthetic construct
SYGMH

SEQ ID NO: 26 - protein, 17 aa, synthetic construct
VMWYDGSDRYSADSVKG

SEQ ID NO: 27 - protein, 13 aa, synthetic construct
GYDILTGPDHFDY

SEQ ID NO: 28 - protein, 11 aa, synthetic construct
RASQSISSYLN

SEQ ID NO: 29 - protein, 7 aa, synthetic construct
AASSLQS

SEQ ID NO: 30 - protein, 9 aa, synthetic construct
QQSYSTPLT

SEQ ID NO: 31 - protein, 5 aa, synthetic construct
SYDMN

SEQ ID NO: 32 - protein, 17 aa, synthetic construct
TISGSGSHTYYADSVRG

SEQ ID NO: 33 - protein, 9 aa, synthetic construct
EGGATAFDI

SEQ ID NO: 34 - protein, 11 aa, synthetic construct
RASQGISSYLA

SEQ ID NO: 35 - protein, 7 aa, synthetic construct
VASTLQS

SEQ ID NO: 36 - protein, 8 aa, synthetic construct
QQLNNYPT

SEQ ID NO: 37 - protein, 5 aa, synthetic construct
TYGMH

SEQ ID NO: 38 - protein, 17 aa, synthetic construct
LIWYDGINKYYADSVKG

SEQ ID NO: 39 - protein, 12 aa, synthetic construct
PYYDILTGYFDY

SEQ ID NO: 40 - protein, 12 aa, synthetic construct
RASQSDSSSYLA

SEQ ID NO: 41 - protein, 7 aa, synthetic construct
GTSSRAT

SEQ ID NO: 42 - protein, 9 aa, synthetic construct
QQYGSSPLT

SEQ ID NO: 43 - protein, 17 aa, synthetic construct
VIWYDGTNKYYADSVKG

SEQ ID NO: 44 - protein, 17 aa, synthetic construct
DPSLWFGEFPHYYGMDV

SEQ ID NO: 45 - protein, 9 aa, synthetic construct
QQYDNLPLT

SEQ ID NO: 46 - protein, 5 aa, synthetic construct
GYYWS

SEQ ID NO: 47 - protein, 16 aa, synthetic construct
EIQHSGSTNYKPSLKS

SEQ ID NO: 48 - protein, 10 aa, synthetic construct
LTGDSLLFEY

SEQ ID NO: 49 - protein, 11 aa, synthetic construct
RASQSVSSYLA

SEQ ID NO: 50 - protein, 7 aa, synthetic construct
DTSNRAT

SEQ ID NO: 51 - protein, 9 aa, synthetic construct
QQRSNWPIT

SEQ ID NO: 52 - protein, 17 aa, synthetic construct
VIWYDGSNKYYADSVKG

SEQ ID NO: 53 - protein, 9 aa, synthetic construct
DSASDYFDY

SEQ ID NO: 54 - protein, 11 aa, synthetic construct
RASQSVSSNLA

SEQ ID NO: 55 - protein, 8 aa, synthetic construct
QQYSDWPT

SEQ ID NO: 56 - protein, 16 aa, synthetic construct
EINHSGSTNYKPSLKS

SEQ ID NO: 57 - protein, 107 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQQYDNLPITFGQGTRLEIK

SEQ ID NO: 58 - protein, 126 aa, synthetic construct
EVQLLESGGGLVQPGGSLRLSCAASGFTFSSYAMSWVRQAPGKGLEWVSAISDSGGSTYYADSVKGRFTISRDNSKNTLYLQMNSLRAEDTAVYYCAQEGLITFGGVIVIGYFDYWGQGTLVTVSS

SEQ ID NO: 59 - protein, 107 aa, synthetic construct
EIVLTQSPATLSLSPGERATLSCRASQYVSSYLAWYHQKPGQAPRLLIYDASNRATGIPARFSGSGSGTDFTLTISSLEPEDFAVYYCQQRNNWPPTFGQGTKVEIK

SEQ ID NO: 60 - protein, 117 aa, synthetic construct
EVQLVESGGGLVQPGGSLRLSCAASGFTFSSYWMSWVRQAPGKGLEWVANIKQDGSQKYYVDSVKGRFTISRDNAKNSLYLQMNSLRAEDTAVYYCTRRGNFFFDNWGQGTLVTVSS

SEQ ID NO: 61 - protein, 107 aa, synthetic construct
DIQMTQSPSSLSASVGDRVTITCRASQGITNYLAWYQQKPGKVPKLLIYAASTLQSGVPSRFSGSGSGTDFTLTINSLQPEDVATYYCQKYNSAPWTFGQGTKVEIK

SEQ ID NO: 62 - protein, 112 aa, synthetic construct
QVQLQESGPGLVKPSETLSLTCTVSGGSISNHYWSWIRQPPGKGLEWIGYIYYSGSTNYNPSLKSRVTISVDTSKNQFSLKLSSVTAADTAVYYCARGFAYWGQGTLVTVSS

SEQ ID NO: 63 - protein, 107 aa, synthetic construct
EIVMTLSPATLSVSPGERATLSCRASQSINSNLAWYQQKPGQAPRLLIYGASTRATGIPARFSGSGSGTEFTLTISSLQSEDFAVYYCQQYNNWPLTFGGGTKVEIK

SEQ ID NO: 64 - protein, 125 aa, synthetic construct
EVQLLESGGGLGQPGGSLRLSCAASGFTFSSYAMNWVRQAPGKGLEWVSAISGSGDSTYSTDSVKGRFTISRDNSKNTLYLQMNSLRAEDTAVYYCAKEYYDILTGYWDWYFDLWGQGTLVTVSS

SEQ ID NO: 65 - protein, 107 aa, synthetic construct [sequence omitted]

SEQ ID NO: 66 - protein, 122 aa, synthetic construct
QVQLVESGGGVVQPGRSLRLSCAASGFTFSSYGMHWVRQAPGKGLEWVAVMWYDGSDRYSADSVKGRFTISRDNSKNTLYLQMNSLRAEDTAVYYCARGYDILTGPDHFDYWGQGTLVTVSS

SEQ ID NO: 67 - protein, 106 aa, synthetic construct
DIQLTQSPSFLSASVGDRVTITCRASQGISSYLAWYQQKPGKAPKLLIYVASTLQSGVPSRFSGSGSGTEFTLTISSLQPEDFATYYCQQLNNYPTFGGGTKVEIK

SEQ ID NO: 68 - protein, 118 aa, synthetic construct
EVQLLESGGGLVQPGGSLRLSCAASGFTFSSYDMNWVRQAPGKGPVWVSTISGSGSHTYYADSVRGRFTISRDNSKNTLYLQMNSLRAEDTAVYYCAKEGGATAFDIWGQGTMVTVSS

SEQ ID NO: 69 - protein, 108 aa, synthetic construct
EIVLTQSPGTLSLSPGERATLSCRASQSDSSSYLAWYQQKPGQAPRLLIYGTSSRATGISDRFSGSGSGTDFTLTISRLEPEDFAVYYCQQYGSSPLTFGGGTKVEIK

SEQ ID NO: 70 - protein, 121 aa, synthetic construct
QVQLVESGGGVVQPGRSLRLSCAASGFTFSTYGMHWVRQAPGKGLEWVALIWYDGINKYYADSVKGRFTISRDNSKNTLYLQMNSLRAEDTAVYFCARPYYDILTGYFDYWGQGTLVTVSS

SEQ ID NO: 71 - protein, 107 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQQYDNLPLTFGGGTKVEIK

SEQ ID NO: 72 - protein, 126 aa, synthetic construct
QVQLVESGGGVVQPGRSLRLSCAASGFTFSSYGMHWVRQAPGKGLEWVAVIWYDGTNKYYADSVKGRFTISRDNSKNTLYLQVNSLRAEDTAVYYCARDPSLWFGEFPHYYGMDVWGQGTTVTVSS

SEQ ID NO: 73 - protein, 107 aa, synthetic construct
EIVLTQSPATLSLSPGERATLSCRASQSVSSYLAWYQQKPGQAPRLLIYDTSNRATGIPARFSGSGSGTDFTLTVSSLEPEDFAVYYCQQRSNWPITFGQGTRLEIK

SEQ ID NO: 74 - protein, 118 aa, synthetic construct
QVQLQQWGAGLLKPSETLSLTCAVYGGSFSGYYWSWIRQPPGKGLEWIGEIQHSGSTNYKPSLKSRVTISVDTSKNQFSLKLSSVTAADTAVYYCAELTGDSLLFEYWGQGTLVTVSS

SEQ ID NO: 75 - protein, 106 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYSDWPTFGGGTKVEIR

SEQ ID NO: 76 - protein, 118 aa, synthetic construct
QVQLVESGGGVVQPGRSLRLSCAASGFTFSSYGMHWVRQAPGKGLEWVAVIWYDGSNKYYADSVKGRFTISRDTSKNTLYLQMNSLRAEDTAVYYCARDSASDYFDYWGQGTLVTVSS

SEQ ID NO: 77 - protein, 118 aa, synthetic construct
QVQLQQWGAGLLKPSETLSLTCAVYGGSFSGYYWSWIRQPPGKGLEWIGEINHSGSTNYKPSLKSRVTISVDTSKNQFSLKLSSVTAADTAVYYCAELTGDSLLFEYWGQGTLVTVSS

SEQ ID NO: 78 - DNA, 15 bp, synthetic construct
  source 1..15: mol_type = other DNA
agctatgccatgagc

SEQ ID NO: 79 - DNA, 51 bp, synthetic construct
  source 1..51: mol_type = other DNA
gctattagtgatagtggtggtagcacatactacgcagactccgtgaagggc

SEQ ID NO: 80 - DNA, 51 bp, synthetic construct
  source 1..51: mol_type = other DNA
gagggtttgattacgtttgggggagttatcgttataggctactttgactac

SEQ ID NO: 81 - DNA, 33 bp, synthetic construct
  source 1..33: mol_type = other DNA
caggcgagtcaggacattagcaactatttaaat

SEQ ID NO: 82 - DNA, 21 bp, synthetic construct
  source 1..21: mol_type = other DNA
gatgcatccaatttggaaaca

SEQ ID NO: 83 - DNA, 27 bp, synthetic construct
  source 1..27: mol_type = other DNA
caacagtatgataatctcccgatcacc

SEQ ID NO: 84 - DNA, 15 bp, synthetic construct
  source 1..15: mol_type = other DNA
agctattggatgagc

SEQ ID NO: 85 - DNA, 51 bp, synthetic construct
  source 1..51: mol_type = other DNA
aatataaagcaagatggaagtcagaaatactatgtggactctgtgaagggc

SEQ ID NO: 86 - DNA, 24 bp, synthetic construct
  source 1..24: mol_type = other DNA
cgtgggaacttcttctttgacaat

SEQ ID NO: 87 - DNA, 33 bp, synthetic construct
  source 1..33: mol_type = other DNA
agggccagtcagtatgttagcagctacttagcc

SEQ ID NO: 88 - DNA, 21 bp, synthetic construct
  source 1..21: mol_type = other DNA
gatgcatccaacagggccact

SEQ ID NO: 89 - DNA, 27 bp, synthetic construct
  source 1..27: mol_type = other DNA
cagcagcgtaacaactggcctccgacg

SEQ ID NO: 90 - DNA, 15 bp, synthetic construct
  source 1..15: mol_type = other DNA
aatcactactggagc

SEQ ID NO: 91 - DNA, 48 bp, synthetic construct
  source 1..48: mol_type = other DNA
tatatctattacagtgggagcaccaactacaacccctccctcaagagt

SEQ ID NO: 92 - DNA, 12 bp, synthetic construct
  source 1..12: mol_type = other DNA
gggtttgcttac

SEQ ID NO: 93 - DNA, 33 bp, synthetic construct
  source 1..33: mol_type = other DNA
cgggcgagtcagggcattaccaattatttagcc

SEQ ID NO: 94 - DNA, 21 bp, synthetic construct
  source 1..21: mol_type = other DNA
gctgcatccactttgcaatca

SEQ ID NO: 95 - DNA, 27 bp, synthetic construct
  source 1..27: mol_type = other DNA
caaaagtataacagtgccccgtggacg

SEQ ID NO: 96 - DNA, 15 bp, synthetic construct
  source 1..15: mol_type = other DNA
agctatgccatgaac

SEQ ID NO: 97 - DNA, 51 bp, synthetic construct
  source 1..51: mol_type = other DNA
gctatcagtggcagtggtgatagcacatactccacagactccgtgaagggc

SEQ ID NO: 98 - DNA, 48 bp, synthetic construct
  source 1..48: mol_type = other DNA
gagtattacgatattttgactggttattgggactggtacttcgatctc

SEQ ID NO: 99 - DNA, 33 bp, synthetic construct
  source 1..33: mol_type = other DNA
agggccagtcagagtattaacagcaacttagcc

SEQ ID NO: 100 - DNA, 21 bp, synthetic construct
  source 1..21: mol_type = other DNA
ggtgcatccaccagggccact

SEQ ID NO: 101 - DNA, 27 bp, synthetic construct
  source 1..27: mol_type = other DNA
cagcagtataataactggccgctcact

SEQ ID NO: 102 - DNA, 15 bp, synthetic construct
  source 1..15: mol_type = other DNA
agctatggcatgcat

SEQ ID NO: 103 - DNA, 51 bp, synthetic construct
  source 1..51: mol_type = other DNA
gttatgtggtatgatggaagtgatagatactctgcagactccgtgaagggc

SEQ ID NO: 104 - DNA, 39 bp, synthetic construct
  source 1..39: mol_type = other DNA
gggtacgatattttgactggtcccgaccactttgactac

SEQ ID NO: 105 - DNA, 33 bp, synthetic construct
  source 1..33: mol_type = other DNA
cgggcaagtcagagcattagcagttatttaaat

SEQ ID NO: 106 - DNA, 21 bp, synthetic construct
  source 1..21: mol_type = other DNA
gctgcatccagtttgcaaagt

SEQ ID NO: 107 - DNA, 27 bp, synthetic construct
  source 1..27: mol_type = other DNA
caacagagttacagtaccccgctcact

SEQ ID NO: 108 - DNA, 15 bp, synthetic construct
  source 1..15: mol_type = other DNA
agctatgacatgaac

SEQ ID NO: 109 - DNA, 51 bp, synthetic construct
  source 1..51: mol_type = other DNA
actattagtggtagtggtagtcacacatactacgcagactccgtgaggggc

SEQ ID NO: 110 - DNA, 27 bp, synthetic construct
  source 1..27: mol_type = other DNA
gaggggggagctactgcttttgatatc

SEQ ID NO: 111 - DNA, 33 bp, synthetic construct
  source 1..33: mol_type = other DNA
cgggccagtcagggcattagcagttatttagcc

SEQ ID NO: 112 - DNA, 21 bp, synthetic construct
  source 1..21: mol_type = other DNA
gttgcatccactttgcaaagt

SEQ ID NO: 113 - DNA, 24 bp, synthetic construct
  source 1..24: mol_type = other DNA
caacagcttaataattaccccact

SEQ ID NO: 114 - DNA, 15 bp, synthetic construct
  source 1..15: mol_type = other DNA
acctatggcatgcac

SEQ ID NO: 115 - DNA, 51 bp, synthetic construct
  source 1..51: mol_type = other DNA
cttatatggtatgatggaattaataaatactatgcggactccgtgaagggc

SEQ ID NO: 116 - DNA, 36 bp, synthetic construct
  source 1..36: mol_type = other DNA
ccctattacgatattttgactggttattttgactac

SEQ ID NO: 117 - DNA, 36 bp, synthetic construct
  source 1..36: mol_type = other DNA
agggccagtcagagtgatagcagcagctacttagcc

SEQ ID NO: 118 - DNA, 21 bp, synthetic construct
  source 1..21: mol_type = other DNA
ggtacatccagtagggccact

SEQ ID NO: 119 - DNA, 27 bp, synthetic construct
  source 1..27: mol_type = other DNA
cagcagtatggtagctcaccgctcact

SEQ ID NO: 120 - DNA, 15 bp, synthetic construct
  source 1..15: mol_type = other DNA
agctatggcatgcac

SEQ ID NO: 121 - DNA, 51 bp, synthetic construct
  source 1..51: mol_type = other DNA
gttatatggtatgatggaactaataaatactatgcagactccgtgaagggc

SEQ ID NO: 122 - DNA, 51 bp, synthetic construct
  source 1..51: mol_type = other DNA
gatccctccttatggttcggggagttccctcattactacggtatggacgtc

SEQ ID NO: 123 - DNA, 33 bp, synthetic construct
  source 1..33: mol_type = other DNA
caggcgagtcaggacattagcaattatttaaat

SEQ ID NO: 124 - DNA, 27 bp, synthetic construct
  source 1..27: mol_type = other DNA
caacagtatgataatctcccgctcact

SEQ ID NO: 125 - DNA, 15 bp, synthetic construct
  source 1..15: mol_type = other DNA
ggttactactggagc

SEQ ID NO: 126 - DNA, 48 bp, synthetic construct
  source 1..48: mol_type = other DNA
gaaatccaacatagtggaagcaccaactacaaaccgtccctcaagagt

SEQ ID NO: 127 - DNA, 30 bp, synthetic construct
  source 1..30: mol_type = other DNA
ctaactggggattcccttttgtttgagtac

SEQ ID NO: 128 - DNA, 33 bp, synthetic construct
  source 1..33: mol_type = other DNA
agggccagtcagagtgttagcagctacttagcc

SEQ ID NO: 129 - DNA, 21 bp, synthetic construct
  source 1..21: mol_type = other DNA
gatacatccaacagggccact

SEQ ID NO: 130 - DNA, 27 bp, synthetic construct
  source 1..27: mol_type = other DNA
cagcagcgtagcaactggccgatcacc

SEQ ID NO: 131 - DNA, 51 bp, synthetic construct
  source 1..51: mol_type = other DNA
gttatatggtatgatggaagtaataaatactatgcagactccgtgaagggc

SEQ ID NO: 132 - DNA, 27 bp, synthetic construct
  source 1..27: mol_type = other DNA
gatagcgcctccgactactttgactac

SEQ ID NO: 133 - DNA, 33 bp, synthetic construct
  source 1..33: mol_type = other DNA
agggccagtcagagtgttagcagcaacttagcc

SEQ ID NO: 134 - DNA, 24 bp, synthetic construct
  source 1..24: mol_type = other DNA
cagcagtatagtgactggcccact

SEQ ID NO: 135 - DNA, 48 bp, synthetic construct
  source 1..48: mol_type = other DNA
gaaatcaatcatagtggaagcaccaactacaaaccgtccctcaagagt

SEQ ID NO: 136 - DNA, 321 bp, synthetic construct
  source 1..321: mol_type = other DNA
gacatccagatgacccagtctccatcctccctgtctgcatctgtaggagacagagtcaccatcacttgccaggcgagtcaggacattagcaactatttaaattggtatcagcagaaaccagggaaagcccctaagctcctgatctacgatgcatccaatttggaaacaggggtcccatcaaggttcagtggaagtggatctgggacagattttactttcaccatcagcagcctgcagcctgaagatattgcaacatattactgtcaacagtatgataatctcccgatcaccttcggccaagggacacgactggagattaaa

SEQ ID NO: 137 - DNA, 378 bp, synthetic construct
  source 1..378: mol_type = other DNA
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtgatagtggtggtagcacatactacgcagactccgtgaagggccggttcaccatctccagagacaattccaagaacacgctgtatctgcaaatgaacagcctgagagccgaggacacggccgtatattactgtgcgcaggagggtttgattacgtttgggggagttatcgttataggctactttgactactggggccagggaaccctggtcaccgtctcctca

SEQ ID NO: 138 - DNA, 321 bp, synthetic construct
  source 1..321: mol_type = other DNA
gaaattgtgttgacacagtctccagccaccctgtctttgtctccaggggaaagagccaccctctcctgcagggccagtcagtatgttagcagctacttagcctggtaccaccagaaacctggccaggctcccaggctcctcatctacgatgcatccaacagggccactggcatcccagccaggttcagtggcagtgggtctgggacagacttcactctcaccatcagcagcctagagcctgaagattttgcagtttattactgtcagcagcgtaacaactggcctccgacgttcggccaagggaccaaggtggaaatcaaa

SEQ ID NO: 139 - DNA, 351 bp, synthetic construct
  source 1..351: mol_type = other DNA
gaggtgcagctggtggagtctgggggaggcttggtccagcctggggggtccctgagactctcctgtgcagcctctggattcacctttagtagctattggatgagctgggtccgccaggctccagggaaggggctggaatgggtggccaatataaagcaagatggaagtcagaaatactatgtggactctgtgaagggccgattcaccatctccagagacaacgccaagaactcactgtatctgcaaatgaacagcctgagagccgaggacacggctgtgtattactgtacgagacgtgggaacttcttctttgacaattggggccagggaaccctggtcaccgtctcctca

SEQ ID NO: 140 - DNA, 321 bp, synthetic construct
  source 1..321: mol_type = other DNA
gacatccagatgacccagtctccatcctccctgtctgcatctgtaggagacagagtcaccatcacttgccgggcgagtcagggcattaccaattatttagcctggtatcagcagaaaccagggaaagttcctaagctcctgatctatgctgcatccactttgcaatcaggggtcccatctcggttcagtggcagtggatctgggacagatttcactctcaccatcaacagcctgcagcctgaagatgttgcaacttattactgtcaaaagtataacagtgccccgtggacgttcggccaagggaccaaggtggaaatcaaa

SEQ ID NO: 141 - DNA, 336 bp, synthetic construct
  source 1..336: mol_type = other DNA
caggtgcagctgcaggagtcgggcccaggactggtgaagccttcggagaccctgtccctcacttgcactgtctctggtggctccatcagtaatcactactggagctggattcggcagcccccagggaagggactggagtggattgggtatatctattacagtgggagcaccaactacaacccctccctcaagagtcgagtcaccatatcagtagacacgtccaagaaccagttctccctgaagctgagctctgtgaccgctgcggacacggccgtgtattactgtgcgagggggtttgcttactggggccaaggcactctggtcactgtctcttca

SEQ ID NO: 142 - DNA, 321 bp, synthetic construct
  source 1..321: mol_type = other DNA
gaaatagtgatgacgctgtctccagccaccctgtctgtgtctccaggggaaagagccaccctctcctgcagggccagtcagagtattaacagcaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtataataactggccgctcactttcggcggagggaccaaggtggagatcaaa

SEQ ID NO: 143 - DNA, 375 bp, synthetic construct
  source 1..375: mol_type = other DNA
gaggtgcagctgttggaatctgggggaggcttgggacagcctggggggtccctgagactctcctgtgcagcctctggattcacctttagcagctatgccatgaactgggtccgccaggctccagggaaggggctggagtgggtctcagctatcagtggcagtggtgatagcacatactccacagactccgtgaagggccggttcaccatctccagagacaattccaagaacacgctgtatctgcaaatgaacagcctgagagccgaggacacggccgtttattactgtgcgaaagagtattacgatattttgactggttattgggactggtacttcgatctctggggccaaggcaccctggtcactgtctcctca

SEQ ID NO: 144 - DNA, 321 bp, synthetic construct
  source 1..321: mol_type = other DNA
gacatccagatgacccagtctccatcctccctgtctgcatctgtaggagacagagtcaccatcacttgccgggcaagtcagagcattagcagttatttaaattggtatcagcagaaaccagggaaagcccctaagctcctgatctatgctgcatccagtttgcaaagtggggtcccatcgaggttcagtggcagtggatctgggacagatttcactctcaccatcagcagtctgcaacctgaagattttgcaacttactattgtcaacagagttacagtaccccgctcactttcggcggagggaccaaggtggagatcaaa

SEQ ID NO: 145 - DNA, 366 bp, synthetic construct
  source 1..366: mol_type = other DNA
caggtgcagctggtggagtctgggggaggcgtggtccagcctgggaggtccctgagactctcctgtgcagcgtctggattcaccttcagtagctatggcatgcattgggtccgccaggctccaggcaaggggctggagtgggtggcagttatgtggtatgatggaagtgatagatactctgcagactccgtgaagggccgattcaccatctccagagacaattccaagaacacgctgtatttgcaaatgaacagcctgagagccgaggacacggctgtgtattactgtgcgagggggtacgatattttgactggtcccgaccactttgactactggggccagggaaccctggtcaccgtctcctca

SEQ ID NO: 146 - DNA, 318 bp, synthetic construct
  source 1..318: mol_type = other DNA
gacatccagttgacccagtctccatccttcctgtctgcatctgtaggagacagagtcaccatcacttgccgggccagtcagggcattagcagttatttagcctggtatcagcaaaaaccagggaaagcccctaagctcctgatctatgttgcatccactttgcaaagtggggtcccatcaaggttcagcggcagtggatctgggacagaattcactctcacaatcagcagcctgcagcctgaagattttgcaacttattactgtcaacagcttaataattaccccactttcggcggagggaccaaggtggagatcaaa

SEQ ID NO: 147 - DNA, 354 bp, synthetic construct
  source 1..354: mol_type = other DNA
gaagtacagttgttggagtctgggggaggcttggtacagcctggggggtccctgagactctcctgtgcagcctctggattcacctttagcagctatgacatgaactgggtccgccaggctccagggaaggggccggtgtgggtctcaactattagtggtagtggtagtcacacatactacgcagactccgtgaggggccggttcaccatctccagagacaactccaagaacacgctgtatctgcaaatgaacagcctgagagccgaggacacggccgtatattactgtgcgaaagaggggggagctactgcttttgatatctggggccaagggacaatggtcaccgtctcttca

SEQ ID NO: 148 - DNA, 324 bp, synthetic construct
  source 1..324: mol_type = other DNA
gaaattgtgttgacgcagtctccaggcaccctgtctttgtctccaggggaaagagccaccctctcctgcagggccagtcagagtgatagcagcagctacttagcctggtatcagcagaaacctggccaggctcccaggctcctcatatatggtacatccagtagggccactggcatctcagacaggttcagtggcagtgggtctgggacagacttcactctcaccatcagcagactggagcctgaagattttgcagtgtattactgtcagcagtatggtagctcaccgctcactttcggcggagggaccaaggtggagatcaaa

SEQ ID NO: 149 - DNA, 363 bp, synthetic construct
  source 1..363: mol_type = other DNA
caggtgcagctggtggagtctgggggaggcgtggtccagcctgggaggtccctgagactctcctgtgcagcgtctggattcaccttcagtacctatggcatgcactgggtccgccaggctccaggcaaggggctggagtgggtggcacttatatggtatgatggaattaataaatactatgcggactccgtgaagggccgattcaccatctccagagacaattccaagaacacgctgtatctgcaaatgaacagcctgagagccgaggacacggctgtgtatttctgtgcgagaccctattacgatattttgactggttattttgactactggggccagggaaccctggtcaccgtctcctca

SEQ ID NO: 150 - DNA, 321 bp, synthetic construct
  source 1..321: mol_type = other DNA
gacatccagatgacccagtctccatcctccctgtctgcatctgtaggagacagagtcaccatcacttgccaggcgagtcaggacattagcaattatttaaattggtatcagcagaaaccagggaaagcccctaagctcctgatctacgatgcatccaatttggaaacaggggtcccatcaaggttcagtggaagtggatctgggacagattttactttcaccatcagcagcctgcagcctgaagatattgcaacatattactgtcaacagtatgataatctcccgctcactttcggcggagggaccaaggtggagatcaaa

SEQ ID NO: 151 - DNA, 378 bp, synthetic construct
  source 1..378: mol_type = other DNA
caggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatggtatgatggaactaataaatactatgcagactccgtgaagggccgattcaccatctccagagacaattccaagaacacgctgtatctgcaagtgaacagcctgagagccgaggacacggctgtgtattactgtgcgagagatccctccttatggttcggggagttccctcattactacggtatggacgtctggggccaagggaccacggtcaccgtctcctca

SEQ ID NO: 152 - DNA, 321 bp, synthetic construct
  source 1..321: mol_type = other DNA
gaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatacatccaacagggccactggcatcccagccaggttcagtggcagtgggtctgggacagacttcactctcaccgtcagcagcctagagcctgaagattttgcagtttattactgtcagcagcgtagcaactggccgatcaccttcggccaagggacacgactggagattaaa

SEQ ID NO: 153 - DNA, 354 bp, synthetic construct
  source 1..354: mol_type = other DNA
caggtgcagctacagcagtggggcgcaggactgttgaagccttcggagaccctgtccctcacctgcgctgtctatggtgggtccttcagtggttactactggagctggatacgccagcccccagggaaggggctggagtggattggggaaatccaacatagtggaagcaccaactacaaaccgtccctcaagagtcgagtcaccatatcagtagacacgtccaagaaccagttctccctgaagctgagctctgtgaccgccgcggacacggctgtgtattactgtgcggagctaactggggattcccttttgtttgagtactggggccagggaaccctggtcaccgtctcctca

SEQ ID NO: 154 - DNA, 318 bp, synthetic construct
  source 1..318: mol_type = other DNA
gaaatagtgatgacgcagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccactggtatcccagccagattcagtggcagtgggtctgggacagagttcactctcaccatcagcagcctgcagtctgaagattttgcagtttattactgtcagcagtatagtgactggcccactttcggcggagggaccaaggtggagatcaga

SEQ ID NO: 155 - DNA, 354 bp, synthetic construct
  source 1..354: mol_type = other DNA
caggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatggtatgatggaagtaataaatactatgcagactccgtgaagggccgattcaccatctccagagacacttccaagaacacactgtatctgcaaatgaacagcctgagagccgaggacacggctgtgtattactgtgcgagagatagcgcctccgactactttgactactggggccagggaaccctggtcaccgtctcctca

SEQ ID NO: 156 - DNA, 354 bp, synthetic construct
  source 1..354: mol_type = other DNA
caggtgcagctacagcagtggggcgcaggactgttgaagccttcggagaccctgtccctcacctgcgctgtctatggtgggtccttcagtggttactactggagctggatacgccagcccccagggaaggggctggagtggattggggaaatcaatcatagtggaagcaccaactacaaaccgtccctcaagagtcgagtcaccatatcagtagacacgtccaagaaccagttctccctgaagctgagctctgtgaccgccgcggacacggctgtgtattactgtgcggagctaactggggattcccttttgtttgagtactggggccagggaaccctggtcaccgtctcctca